(19) United States
(12) Patent Application Publication
(10) Pub. No.: US 2009/0326018 A1
(43) Pub. Date: Dec. 31, 2009

(54) 2-Phenyl indene derivatives useful as estrogen receptor ligands

(76) Inventors: Henrik Jernstedt, Uppsala (SE); Lars Krüger, Huddinge (SE); Patrik Rhönnstad, Kungsor (SE); Daniel Nöteberg, Vasterhaninge (SE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 12/311,722

(22) PCT Filed: Oct. 12, 2007
(86) PCT No.: PCT/EP2007/008884

§ 371 Date: Jun. 1, 2009

(30) Foreign Application Priority Data
Oct. 13, 2006 (GB) 0620397.0
Apr. 20, 2007 (GB) 0707721.7

(51) Int. Cl. A61K 31/425; C07D 307/02; A61K 31/341; C07D 233/90; A61K 31/4164; C07C 39/12; A61K 31/05; C07D 277/20

(52) U.S. Cl. 514/365; 549/474; 514/471; 548/337.1; 514/400; 568/717; 514/732; 548/202

(57) ABSTRACT

The invention provides a compound of formula (I) or a pharmaceutically acceptable ester, amide, solvate or salt thereof, including a salt of such an ester or amide, and a solvate of such an ester, amide or salt. The invention also provides also provides the use of such compounds in the treatment or prophylaxis of a condition associated with a disease or disorder associated with estrogen receptor activity. Formula (I), wherein R¹, R², R³, R⁴, R⁵, R⁶, R⁷, R⁸, R⁹, R¹⁰, R¹¹ and R¹² are as defined in the specification, and wherein either the bond between the C1 and C2 carbon atoms is a double bond or the bond between the C2 and C3 carbon atoms is a double bond, R² being absent when the bond between the C1 and C2 carbon atoms is a double bond.

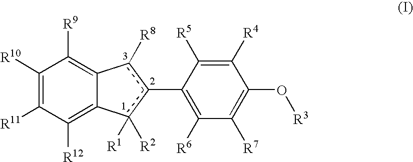

### FIELD OF INVENTION

This invention relates to compounds which are estrogen receptor ligands and are preferably selective for the estrogen receptor β isoform, to methods of preparing such compounds and to methods for using such compounds in treatment of diseases related to the estrogen receptor such as depressive disorders, anxiety disorders, Alzheimer's disease, cognitive disorders, osteoporosis, elevated blood triglyceride levels, atherosclerosis, endometriosis, urinary incontinence, autoimmune disease, and cancer of the lung, colon, breast, uterus and prostate.

### BACKGROUND OF INVENTION

The estrogen receptor (ER) is a ligand activated mammalian transcription factor involved in the up and down regulation of gene expression. The natural hormone for the estrogen receptor is β-17-estradiol (E2) and closely related metabolites. Binding of estradiol to the estrogen receptor causes a dimerization of the receptor and the dimer in turn binds to estrogen response elements (ERE's) on DNA. The ER/DNA complex recruits other transcription factors responsible for the transcription of DNA downstream from the ERE into mRNA which is eventually is translated into protein. Alternatively the interaction of ER with DNA may be indirect through the intermediacy of other transcription factors, most notably fos and jun. Since the expression of a large number of genes is regulated by the estrogen receptor and since the estrogen receptor is expressed in many cell types, modulation of the estrogen receptor through binding of either natural hormones or synthetic ER ligands can have profound effects on the physiology and pathophysiology of the organism.

Historically it has been believed there was only one estrogen receptor. However a second subtype (ER-β) has been discovered. While both the “classical” ER-α and the more recently discovered ER-β are widely distributed in different tissues, they nevertheless display markedly different cell type and tissue distributions. Therefore synthetic ligands which are either ER-α or ER-β selective may preserve the beneficial effects of estrogen while reducing the risk of undesirable side effects.

Estrogens are critical for sexual development in females. In addition, estrogens play an important role in maintaining bone density, regulation of blood lipid levels, and appear to have neuroprotective effects. Consequently decreased estrogen production in post-menopausal women is associated with a number of diseases such as osteoporosis, atherosclerosis, depression and cognitive disorders. Conversely certain types of proliferative diseases such as breast and uterine cancer and endometriosis are stimulated by estrogens and therefore antiestrogens (i.e., estrogen antagonists) have utility in the prevention and treatment of these types of disorders.

The efficacy of the natural estrogen, 17β-estradiol, for the treatment of various forms of depressive illness has also been demonstrated and it has been suggested that the anti-depressant activity of estrogen may be mediated via regulation of tryptophan hydroxylase activity and subsequent serotonin synthesis (See, e.g., Lu N Z, Shlaes T A, Cundlah C, Dziennis S E, Lyle R E, Bethea C L, “Ovarian steroid action on tryptophan hydroxylase protein and serotonin compared to localization of ovarian steroid receptors in midbrain of guinea pigs.” Endocrine 11:257-267, 1999). The pleiotropic nature of natural estrogen preclude its widespread, more chronic use due to the increased risk of proliferative effects on breast, uterine and ovarian tissues. The identification of the estrogen receptor, ERβ, has provided a means by which to identify more selective estrogen agents which have the desired anti-depressant activity in the absence of the proliferative effects which are mediated by ERα. Thus, it has been shown that therapeutic agents having ERβ-selectivity are potentially particularly effective in the treatment of depression.

What is needed in the art are compounds that can produce the same positive responses as estrogen replacement therapy without the negative side effects. Also needed are estrogen-like compounds that exert selective effects on different tissues of the body.

The compounds of the instant invention are ligands for estrogen receptors and as such may be useful for treatment or prevention of a variety of conditions related to estrogen functioning including bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression, autoimmune disease, and lung, colon, breast, uterus, and prostate cancer.

A description of the synthesis of two series of estrogen receptor ligands is described in N. J. Clegg, S. Paruthiyil, D. C. Leitman and T. S. Scanlan, J. Med. Chem., 2005, 48, 5989-6003. These compounds are based on a common indene scaffold in an attempt to develop compounds that can selectively modulate ER-mediated transcription. The binding affinity of each of the compounds to ERα and ERβ was tested and several compounds were found to differentiate between ERα and ERβ subtypes at an estrogen receptor element (ERE), displaying various levels of partial to full agonist activity at ERα, while antagonizing estradiol action at ERβ.

### SUMMARY OF THE INVENTION

This invention provides a compound of formula (X) or a pharmaceutically acceptable ester, amide, solvate or salt thereof including a salt of such an ester or amide, and a solvate of such an ester, amide or salt,

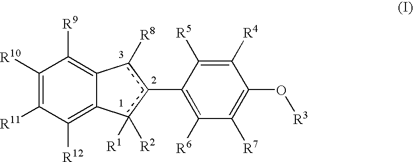

wherein either the bond between the C1 and C2 carbon atoms is a double bond or the bond between the C2 and C3 carbon atoms is a double bond, R² being absent when the bond between the C1 and C2 carbon atoms is a double bond;
R¹ and R² are independently selected from the group consisting of hydrogen, OR^{A}, C₁₋₆ alkyl, C₂₋₆alkenyl, C₂₋₆ alkynyl, C₃₋₈ cycloalkyl, C₃₋₈ cycloalkyl C₁₋₆alkyl, C₆₋₁₀aryl, C₆₋₁₀aryl C₁₋₆alkyl, halogen, halo C₁₋₆ alkyl, dihalo C₁₋₆ alkyl and trihalo C₁₋₆ alkyl; or R¹ and R² taken together with the carbon atom to which they are attached form a double bond portion of C₂₋₆ alkenyl group;
R^{A} is selected from the group consisting of hydrogen, C₁₋₆ alkyl, C₂₋₆ alkenyl, C₂₋₆ alkynyl, C₃₋₈ cycloalkyl, C₃₋₈ cycloalkyl C₁₋₆alkyl, C₆₋₁₀aryl and C₆₋₁₀aryl C₁₋₆alkyl;
R³ is selected from the group consisting of hydrogen, C₁₋₆alkyl, C₃₋₈ cycloalkyl and —C(O)C₁₋₄ alkyl;
R⁴, R⁵, R⁶ and R⁷ are the same or are different and each is selected from the group consisting of hydrogen, OR^{A}, halogen, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl;
R⁸ is selected from the group consisting of C₃₋₈ cycloalkyl, C₃₋₈ cycloalkyl C₁₋₆ alkyl, phenyl, benzyl and C₅₋₁₀ heterocyclyl wherein said phenyl, benzyl or C₅₋₁₀ heterocyclyl group can either be unsubstituted or substituted with 1-3 substituents and each substituent is selected from the group consisting of OR^{A}, halogen, cyano, nitro, C₁₋₆alkyl, C₂₋₆alkenyl, C₂₋₆ alkynyl, halo C₁₋₆alkyl, dihalo C₁₋₆alkyl, trihalo C₁₋₆ alkyl and C(O)C₁₋₆ alkyl;

### R¹⁰ is OR^{A}; and

R⁹, R¹¹ and R¹² are the same or are different and each is selected from the group consisting of hydrogen, OR^{A}, halogen, cyano, nitro, C₁₋₆ alkyl, C₂₋₆ alkenyl, C₂₋₆ alkynyl, C(O)H, C(O)C₁₋₄ alkyl, halo C₁₋₆ alkyl, dihalo C₁₋₄ alkyl and trihalo C₁₋₆ alkyl;
with the proviso that the compound is not 4,5,6-trimethoxy-2-(4-methoxyphenyl)-1-methyl-3-phenyl-1H-indenol, or 3-benzyl-2-(3,4-dimethoxyphenyl)-5,6-dimethoxyindene.

Compounds of the invention have surprisingly been found to be ligands of the estrogen receptor. The compounds accordingly have use in the treatment or prophylaxis of conditions associated with estrogen receptor activity.

4,5,6-Trimethoxy-2-(4-methoxyphenyl)-1-methyl-3-phenyl-1H-indenol is known from Vicente et al., Organometallics, 1996, 15(16), 3509-3519. 3-Benzyl-2-(3,4-dimethoxyphenyl)-5,6-dimethoxyindene is known from Alesso et al., Canadian Journal of Chemistry, 1991, 69(7), 1166-1170.

The invention further provides a compound of formula (I) or a pharmaceutically acceptable ester, amide, solvate or salt thereof, including a salt of such an ester or amide, and a solvate of such an ester, amide or salt,

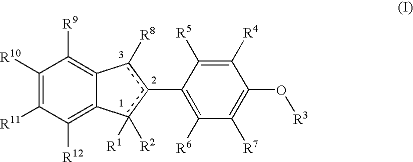

wherein either the bond between the C1 and C2 carbon atoms is a double bond or the bond between the C2 and C3 carbon atoms is a double bond, R² being absent when the bond between the C1 and C2 carbon atoms is a double bond;
R¹ and R² are independently selected from the group consisting of hydrogen, OR^{A}, C₁₋₆alkyl, C₂₋₆ alkenyl, C₂₋₆ alkynyl, C₃₋₈ cycloalkyl, C₃₋₈ cycloalkyl C₁₋₆ alkyl, C₆₋₁₀ aryl, C₆₋₁₀aryl C₁₋₆ alkyl, halogen, halo C₁₋₆ alkyl, dihalo C₁₋₆ alkyl and trihalo C₁₋₆ alkyl; or R¹ and R² taken together with the carbon atom to which they are attached form a double bond portion of C₂₋₆ alkenyl group;
R^{A} is selected from the group consisting of hydrogen, C₁₋₆ alkyl, C₂₋₆ alkenyl, C₂₋₆ alkynyl, C₃₋₈ cycloalkyl, C₃₋₈ cycloalkyl C₁₋₆alkyl, C₆₋₁₀aryl and C₆₋₁₀aryl C₁₋₆alkyl;
R³ is selected from the group consisting of hydrogen, C₁₋₆alkyl, C₃₋₈ cycloalkyl and —C(O)C₁₋₄ alkyl;
R⁴, R⁵, R⁶ and R⁷ are the same or are different and each is selected from the group consisting of hydrogen, OR^{A}, halogen, cyano, nitro, C₁₋₆alkyl, C₂₋₆alkenyl, C₂₋₆ alkynyl, halo C₁₋₆ alkyl, dihalo C₁₋₆alkyl and trihalo C₁₋₆alkyl;
R⁸ is selected from the group consisting of C₃₋₈ cycloalkyl, C₃₋₈ cycloalkyl C₁₋₆ alkyl, phenyl, benzyl and C₅₋₁₀ heterocyclyl wherein said phenyl, benzyl or C₅₋₁₀ heterocyclyl group can either be unsubstituted or substituted with 1-3 substituents and each substituent is selected from the group consisting of OR^{A}, halogen, cyano, nitro, C₁₋₆ alkyl, C₂₋₆ alkenyl, C₂₋₆ alkynyl, halo C₁₋₄ alkyl, dihalo C₁₋₆ alkyl, trihalo C₁₋₆ alkyl and C(O)C₁₋₆ alkyl;

### R¹⁰ is OR^{A}; and

R⁹, R¹¹ and R¹² are the same or are different and each is selected from the group consisting of hydrogen, OR^{A}, halogen, cyano, nitro, C₁₋₆ alkyl, C₂₋₆ alkenyl, C₂₋₆ alkynyl, C(O)H, C(O)C₁₋₆ alkyl, halo C₁₋₆ alkyl, dihalo C₁₋₆ alkyl and trihalo C₁₋₆ alkyl;
for use as a medicament.

### DETAILED DESCRIPTION OF INVENTION

The compounds of formula (I) may contain chiral (asymmetric) centres or the molecule as a whole may be chiral. The individual stereoisomers (enantiomers and diastereoisomers) and mixtures of these are within the scope of the present invention.

The present invention provides compounds that are estrogen receptor ligands and have the general formula (I) as described above. The term “estrogen receptor ligand” as used herein is intended to cover any moiety which binds to an estrogen receptor. The ligand may act as an agonist, a partial agonist, an antagonist or a partial antagonist. The ligand may be ERβ selective or display mixed ERα and ERβ activity. For example, the ligand may act both as an agonist or a partial agonist of ERβ and as an antagonist or a partial antagonist of ERα.

In one embodiment of the invention, the bond between the C1 and C2 carbon atoms is a double bond. In an alternative embodiment of the invention, the bond between the C2 and C3 carbon atoms is a double bond.

Preferably, R¹ and R² are independently selected from the group consisting of hydrogen, OR^{A}, C₁₋₆ alkyl, C₃₋₆ cycloalkyl, C₃₋₆ cycloalkyl C₁₋₄ alkyl, phenyl, benzyl, halogen, halo C₁₋₆ alkyl, dihalo C₁₋₄ alkyl and trihalo C₁₋₆ alkyl. More preferably, R¹ and R² are independently selected from the group consisting of hydrogen, OR^{A}, C₁₋₆ alkyl, halogen, halo C₆₋₄alkyl, dihalo C₁₋₆alkyl and trihalo C₁₋₆ alkyl. Most preferably, R¹ and R² are independently selected from the group consisting of hydrogen and C₁₋₆alkyl. In a particularly preferred embodiment, R¹ and R² are both hydrogen.

Preferably, R^{A} is selected from the group consisting of hydrogen, C₁₋₄ alkyl, C₃₋₆ cycloalkyl, C₃₋₆ cycloalkyl C₁₋₄ alkyl, phenyl and benzyl. More preferably, R^{A} is selected from the group consisting of hydrogen, C₁₋₄ alkyl and C₃₋₆ cycloalkyl. Most preferably, R^{A} is selected from the group consisting of hydrogen and C₁₋₄ alkyl. For Example R^{A} is H.

Preferably, R³ is selected from the group consisting of hydrogen, C₁₋₄ alkyl, C₃₋₆ cycloalkyl and —C(O)C₁₋₄ alkyl. More preferably, R₃ is selected from the group consisting of hydrogen and C₁₋₄ alkyl. Most preferably, R₃ is hydrogen.

R⁴, R⁵, R⁶ and R⁷ are the same or are different and each is preferably selected from the group consisting of hydrogen, OR^{A}, halogen, cyano, nitro, C₁₋₄ alkyl, halo C₁₋₄ alkyl, dihalo C₁₋₄ alkyl and trihalo C₁₋₄ alkyl. More preferably, R₄, R₅, R₆ and R₇ are independently selected from the group consisting of hydrogen, OR^{A}, halogen, cyano, methyl, halomethyl, dihalomethyl and trihalomethyl. Most preferably, R₄, R₅, R₆ and R₇ are independently selected from the group consisting of hydrogen, hydroxy, halogen, cyano, methyl, and trifluoromethyl. Amongst the halogens, there are preferred chlorine, bromine, and fluorine, especially chlorine and bromine.

Preferably R⁸ is selected from the group consisting of C₃₋₈ cycloalkyl, C₃₋₈ cycloalkyl C₁₋₆ alkyl, phenyl, benzyl and C₅₋₁₀heterocyclyl wherein said phenyl, benzyl or C₅₋₁₀ heterocyclyl group can either be unsubstituted or substituted with 1-3 substituents and each substituent is selected from the group consisting of OR^{A}, halogen, cyano, nitro, C₁₋₆alkyl, C₂₋₆ alkenyl, C₂₋₆ alkynyl, halo C₁₋₆ alkyl, dihalo C₁₋₆ alkyl and trihalo C₁₋₆ alkyl;

Preferably, R⁸ is selected from the group consisting of C₃₋₆ cycloalkyl, C₃₋₆ cycloalkyl C₁₋₄ alkyl, phenyl, benzyl and C₅₋₆ heterocyclyl with optional substitution of said phenyl, benzyl or C₅₋₆ heterocyclyl groups as described above. More preferably, R₈ is selected from the group consisting of phenyl, benzyl and C₅₋₆ heterocyclyl with optional substitution of said phenyl, benzyl or C₅₋₆heterocyclyl groups as described above. Most preferably, R₈ is phenyl or C₅ heterocyclyl with optional substitution of said phenyl or C₅ heterocyclyl groups as described above. Preferred C₅ heterocyclyl groups include thiophenyl, thiazolyl, furanyl, pyrazolyl, pyrrolyl, oxazolyl and imidazolyl. Preferred substituents for said phenyl, benzyl or heterocyclyl groups include groups selected from OR^{A}, halogen, cyano, nitro, C₁₋₄ alkyl, halo C₁₋₄ alkyl, dihalo C₁₋₄ alkyl trihalo C₁₋₄ alkyl and C(O)C₁₋₆alkyl, particularly hydroxy, halogen, cyano, methyl, ethyl, propyl, iso-propyl and trifluoromethyl.

Preferably, R¹⁰ is hydroxy.

R⁹, R¹¹ and R¹² are the same or are different and each is preferably selected from the group consisting of hydrogen, OR^{A}, halogen, cyano, nitro, C₁₋₄ alkyl, C₂₋₄ alkenyl, halo C₁₋₄ alkyl, dihalo C₁₋₄ alkyl and trihalo C₁₋₄ alkyl. More preferably, R⁹, R¹¹ and R¹² are independently selected from the group consisting of hydrogen, OR^{A}, halogen, cyano, C₁₋₄ alkyl, C(O)H, C(O)C₁₋₃ alkyl, halomethyl, dihalomethyl and trihalomethyl. Most preferably, R⁹, R¹¹ and R¹² are independently selected from the group consisting of hydrogen, hydroxy, halogen, cyano, methyl, ethyl, propyl, ethenyl and trifluoromethyl. Amongst the halogens, there are preferred chlorine, bromine, and fluorine, especially chlorine and fluorine.

In one embodiment of the invention, R⁹ is hydrogen, R¹¹ is hydrogen, and R¹² is selected from the group consisting of hydroxy and C₁₋₄ alkyl.

Accordingly, in one preferred group of compounds of the invention, either the bond between the C1 and C2 carbon atoms is a double bond or the bond between the C2 and C3 carbon atoms is a double bond;

R¹ and R² are independently selected from the group consisting of hydrogen, OR^{A}, C₁₋₆ alkyl, halogen, halo C₁₋₆alkyl, dihalo C₁₋₆alkyl and trihalo C₁₋₆alkyl;
R^{A} is selected from the group consisting of hydrogen, C₁₋₄ alkyl and C₃₋₆ cycloalkyl;
R³ is selected from the group consisting of hydrogen, C₁₋₄ alkyl, C₃₋₆ cycloalkyl and —C(O)C₁₋₄ alkyl;
R⁴, R⁵, R⁶ and R⁷ are the same or are different and each is selected from the group consisting of hydrogen, OR^{A}, halogen, cyano, nitro, C₁₋₄ alkyl, halo C₁₋₄ alkyl, dihalo C₁₋₄ alkyl and trihalo C₁₋₄ alkyl;
R⁸ is selected from the group consisting of C₃₋₆ cycloalkyl, C₃₋₆ cycloalkyl C₁₋₄ alkyl, phenyl, benzyl and C₅₋₆ heterocyclyl wherein said phenyl, benzyl or C₅₋₆ heterocyclyl group can either be unsubstituted or substituted with 1-3 substituents and each substituent is selected from the group consisting of OR^{A}, halogen, cyano, nitro, C₁₋₄ alkyl, halo C₁₋₄ alkyl, dihalo C₁₋₄ alkyl and trihalo C₁₋₄ alkyl;

### R¹⁰ is OR^{A}; and

R⁹, R¹¹ and R¹² are the same or are different and each is selected from the group consisting of hydrogen, OR^{A}, halogen, cyano, nitro, C₁₋₄ alkyl, halo C₁₋₄ alkyl, dihalo C₁₋₄ alkyl and trihalo C₁₋₄ alkyl.

Compounds of the invention include, but are not limited to, the following:
2-(4-Hydroxy-phenyl)-7-methyl-3-p-tolyl-1H-inden-5-ol (E1);
3-(4-Fluoro-phenyl)-2-(4-hydroxy-phenyl)-7-methyl-1H-inden-5-ol (E2);
3-(4-Hydroxy-2-methyl-phenyl)-2-(4-hydroxy-phenyl)-7-methyl-1H-inden-5-ol (E3);
2-(4-Hydroxy-phenyl)-3-phenyl-1H-inden-5-ol (E4);
2-(4-Hydroxy-phenyl)-3-thiophen-2-yl-1H-inden-5-ol (E5);
7-Ethyl-2-(4-hydroxy-phenyl)-3-phenyl-1H-inden-5-ol (E6);
2-(4-Hydroxy-phenyl)-3-phenyl-7-propyl-1H-inden-5-ol (E7);
2-(4-Hydroxy-phenyl)-7-methyl-3-phenyl-1H-inden-5-ol (E8);
2-(4-Hydroxy-phenyl)-1-phenyl-3H-indene-4,6-diol (E9);
6,7-Difluoro-2-(4-hydroxy-phenyl)-3-phenyl-1H-inden-5-ol (E10);
2-(2-Fluoro-4-hydroxy-phenyl)-7-methyl-3-phenyl-1H-inden-5-ol (E11);
2-(3-Fluoro-4-hydroxy-phenyl)-7-methyl-3-phenyl-1H-inden-5-ol (E12);
2-(3-Chloro-4-hydroxy-phenyl)-7-methyl-3-phenyl-1H-inden-5-ol (E13);
2-(3-Bromo-4-hydroxy-phenyl)-7-methyl-3-phenyl-1H-inden-5-ol (E14);
2-(3,5-Dibromo-4-hydroxy-phenyl)-7-methyl-3-phenyl-3H-inden-5-ol (E15);
2-(2,5-Dichloro-4-hydroxy-phenyl)-7-methyl-3-phenyl-3H-inden-5-ol (E16);
2-(4-Hydroxy-phenyl)-7-methyl-3-thiophen-3-yl-1H-inden-5-ol (E17);
2-(4-Hydroxy-phenyl)-7-methyl-3-thiophen-2-yl-1H-inden-5-ol (E18);
7-Bromo-2-(4-hydroxy-phenyl)-3-phenyl-1H-inden-5-ol (E19);
6-Hydroxy-2-(4-hydroxy-phenyl)-1-phenyl-3H-indene-4-carbonitrile (E20);
6-Hydroxy-2-(4-hydroxy-phenyl)-1-phenyl-3H-indene-4-carbaldehyde (E21);
1-Butyl-2-(4-hydroxy-phenyl)-3-phenyl-1H-inden-5-ol (E22);
2-(4-Hydroxy-phenyl)-7-methyl-3-phenyl-1H-indene-1,5-diol (E23);
2-(4-Hydroxy-phenyl)-7-methyl-1-methylene-3-phenyl-1H-inden-5-ol (E24);
2-(4-Hydroxy-phenyl)-1-methyl-3-phenyl-3H-inden-5-ol (E25);
2-(4-Hydroxy-phenyl)-1-isobutyl-3-phenyl-3H-inden-5-ol (E26):
1-Butyl-2-(4-hydroxy-phenyl)-3-phenyl-3H-inden-5-ol (E27);
1-Ethyl-2-(4-hydroxy-phenyl)-3-phenyl-3H-inden-5-ol (E28);
2-(4-Hydroxy-phenyl)-3-phenyl-1-propyl-3H-inden-5-ol (E29);
2-(4-Hydroxy-phenyl)-1-pentyl-3-phenyl-3H-inden-5-ol (E30);
2†-hydroxy-phenyl)-3-(2-cyano-phenyl)-1H-inden-5-ol (E31);
4-[6-Hydroxy-2-(4-hydroxy-phenyl)-4-methyl-3H-inden-1-yl]-thiophene-3-carbonitrile (E32);
2-(4-Hydroxy-phenyl)-7-methyl-3-(3-methyl-thiophen-2-yl)-1H-inden-5-ol (E33);
2-(2,3-Difluoro-4-hydroxy-phenyl)-7-methyl-3-phenyl-1H-inden-5-ol (E34);
2-(2-Chloro-4-hydroxy-phenyl)-7-methyl-3-phenyl-1H-inden-5-ol (E35);
2-(4-Hydroxy-3-methyl-phenyl)-7-methyl-3-phenyl-1H-inden-5-ol (E36);
2-(4-Hydroxy-2-methyl-phenyl)-7-methyl-3-phenyl-1H-inden-5-ol (E37);
3-[6-Hydroxy-2-(4-hydroxy-phenyl)-4-methyl-3H-inden-1-yl]-thiophene-2-carbonitrile (E38);
2-(4-Hydroxy-phenyl)-7-methyl-3-thiazol-5-yl-1H-inden-5-ol (E39);
3-(2-Ethyl-phenyl)-2-(4-hydroxy-phenyl)-7-methyl-1H-inden-5-ol (E40);
2-(4-Hydroxy-phenyl)-3-(2-isopropyl-phenyl)-7-methyl-1H-inden-5-ol (E41);
3-[2-(3-Fluoro-4-hydroxy-phenyl)-6-hydroxy-4-methyl-3H-inden-1-yl]-thiophene-2-carbonitrile (E42);
3-[2-(3-Chloro-4-hydroxy-phenyl)-6-hydroxy-4-methyl-3H-inden-1-yl]-thiophene-2-carbonitrile (E43);
3-[2-(2,3-Difluoro-4-hydroxy-phenyl)-6-hydroxy-4-methyl-3H-inden-1-yl]-thiophene-2-carbonitrile (E44);
3-[6-Hydroxy-2-(4-hydroxy-phenyl)-4-trifluoromethyl-3H-inden-1-yl]-thiophene-2-carbonitrile (E45);
3-[2-(3-Fluoro-4-hydroxy-phenyl)-6-hydroxy-4-trifluoromethyl-3H-inden-1-yl]-thiophene-2-carbonitrile (E46);
3-[2-(3-Chloro-4-hydroxy-phenyl)-6-hydroxy-4-trifluoromethyl-3H-inden-1-yl]-thiophene-2-carbonitrile (E47);
4-[2-(2,3-Difluoro-4-hydroxy-phenyl)-6-hydroxy-4-methyl-3H-inden-1-yl]-thiophene-3-carbonitrile (E48);
4-[6-Hydroxy-2-(4-hydroxy-phenyl)-4-trifluoromethyl-3H-inden-1-yl]-thiophene-3-carbonitrile (E49);
4-[2-(3-Fluoro-4-hydroxy-phenyl)-6-hydroxy-4-trifluoromethyl-3H-inden-1-yl]-thiophene-3-carbonitrile (E50);
4-[2-(2,3-Difluoro-4-hydroxy-phenyl)-6-hydroxy-4-trifluoromethyl-3H-inden-1-yl]-thiophene-3-carbonitrile (E51);
3-[2-(2,3-Difluoro-4-hydroxy-phenyl)-6-hydroxy-4-trifluoromethyl-3H-inden-1-yl]-thiophene-2-carbonitrile (E52);
3-[2-(2,5-Difluoro-4-hydroxy-phenyl)-6-hydroxy-4-trifluoromethyl-3H-inden-1-yl]-thiophene-2-carbonitrile (E53);
3-[2-(2,6-Difluoro-4-hydroxy-phenyl)-6-hydroxy-4-trifluoromethyl-3H-inden-1-yl]-thiophene-2-carbonitrile (E54);
4-[2-(2,5-Difluoro-4-hydroxy-phenyl)-6-hydroxy-4-trifluoromethyl-3H-inden-1-yl]-thiophene-3-carbonitrile (E55);
2-[6-Hydroxy-2-(4-hydroxy-phenyl)-4-methyl-3H-inden-1-yl]-furan-3-carbonitrile (E56);
2-[5-Bromo-6-hydroxy-2-(4-hydroxy-phenyl)-4-methyl-3H-inden-1-yl]-furan-3-carbonitrile (E57);
2-(4-Hydroxy-phenyl)-3-phenyl-7-trifluoromethyl-1H-inden-5-ol (E58);
2-(2,6-Difluoro-4-hydroxy-phenyl)-7-methyl-3-phenyl-1H-inden-5-ol (E59);
2-(2,5-Difluoro-4-hydroxy-phenyl)-7-methyl-3-phenyl-1H-inden-5-ol (E60);
2-(4-Hydroxy-3-trifluoromethyl-phenyl)-7-methyl-3-phenyl-1H-inden-5-ol (E61);
2-(4-Hydroxy-2,6-dimethyl-phenyl)-7-methyl-3-phenyl-1H-inden-5-ol (E62);
2-(3,5-Dichloro-4-hydroxy-phenyl)-7-methyl-3-phenyl-1H-inden-5-ol (E63);
2-(4-Hydroxy-phenyl)-7-methyl-3-thiazol-2-yl-1H-inden-5-ol (E64);
1-{3-[6-Hydroxy-2-(4-hydroxy-phenyl)-4-methyl-3H-inden-1-yl]-furan-2-yl}-pentan-1-one (E65);
4-[2-(3-Fluoro-4-hydroxy-phenyl)-6-hydroxy-4-methyl-3H-inden-1-yl]-thiophene-3-carbonitrile (E66);
2-[2-(3-Fluoro-4-hydroxy-phenyl)-6-hydroxy-4-methyl-3H-inden-1-yl]-thiophene-3-carbonitrile (E67);
4-[2-(2,5-Difluoro-4-hydroxy-phenyl)-6-hydroxy-4-methyl-3H-inden-1-yl]-thiophene-3-carbonitrile (E68);
2-[2-(2,5-Difluoro-4-hydroxy-phenyl)-6-hydroxy-4-methyl-3H-inden-1-yl]-thiophene-3-carbonitrile (E69);
4-Fluoro-2-(4-hydroxy-phenyl)-3-phenyl-1H-inden-5-ol (E70);
4-Fluoro-2-(3-fluoro-4-hydroxy-phenyl)-3-phenyl-1H-inden-5-ol (E71);
2-(2,3-Difluoro-4-hydroxy-phenyl)-4-fluoro-3-phenyl-1H-inden-5-ol (E72);
4,6-Difluoro-2-(4-hydroxy-phenyl)-3-phenyl-1H-inden-5-ol (E73);
4,6-Difluoro-2-(3-fluoro-4-hydroxy-phenyl)-3-phenyl-1H-inden-5-ol (E74);
2-(2,3-Difluoro-4-hydroxy-phenyl)-4,6-difluoro-3-phenyl-1H-inden-5-ol (E75);
3-[7-Fluoro-6-hydroxy-2-(4-hydroxy-phenyl)-3H-inden-1-yl]-thiophene-2-carbonitrile (E76);
3-[7-Fluoro-2-(3-fluoro-4-hydroxy-phenyl)-6-hydroxy-3H-inden-1-yl]-thiophene-2-carbonitrile (E77);
3-[2-(2,3-Difluoro-4-hydroxy-phenyl)-7-fluoro-6-hydroxy-3H-inden-1-yl]-thiophene-2-carbonitnle (E78);
4-[2-(2,3-Difluoro-4-hydroxy-phenyl)-6-hydroxy-3H-inden-1-yl]-thiophene-3-carbonitrile (E79);
4-[6-Hydroxy-2-(4-hydroxy-phenyl)-3H-inden-1-yl]-thiophene-3-carbonitrile (E80);
2-(4-Hydroxy-phenyl)-3-(2-isobutyl-2H-pyrazol-3-yl)-7-methyl-1H-inden-5-ol (E81);
3-(1-Ethyl-1H-pyrrol-2-yl)-2-(4-hydroxy-phenyl)-7-methyl-1H-inden-5-ol (E82);
2-(4-Hydroxy-phenyl)-7-methyl-3-(1-methyl-1H-imidazol-2-yl)-1H-inden-5-ol (E83);
2-(3-Fluoro-4-hydroxy-phenyl)-7-methyl-3-(2-methyl-2H-pyrazol-3-yl)-1H-inden-5-ol (E84);
2-(2,3-Difluoro-4-hydroxy-phenyl)-7-methyl-3-(2-methyl-2H-pyrazol-3-yl)-1H-inden-5-ol (E85);
2-(4-Hydroxy-phenyl)-7-methyl-3-(2-methyl-2H-pyrazol-3-yl)-1H-inden-5-ol (E86);
3-(2-Ethyl-2H-pyrazol-3-yl)-2-(4-hydroxy-phenyl)-7-methyl-1H-inden-5-ol (E87);
7-Difluoromethyl-2-(4-hydroxy-phenyl)-3-phenyl-1H-inden-5-ol (E88);
7-Fluoromethyl-2-(4-hydroxy-phenyl)-3-phenyl-1H-inden-5-ol (E89);
7-Bromomethyl-2-(4-hydroxy-phenyl)-3-phenyl-1H-inden-5-ol (E90);
2-(4-Hydroxy-phenyl)-3-phenyl-7-vinyl-1H-inden-5-ol (E91));
4-[2-(3-Fluoro-4-methoxy-phenyl)-6-methoxy-4-methyl-3H-inden-1-yl]-thiophene-3-carbonitrile (E92);
4-[4-Fluoro-6-hydroxy-2-(4-hydroxy-phenyl)-3H-inden-1-yl]-thiophene-3-carbonitrile (E93);
4-[2-(2,3-Difluoro-4-hydroxy-phenyl)-4-fluoro-6-hydroxy-3H-inden-1-yl]-thiophene-3-carbonitrile (E94);
4-[5,7-Difluoro-2-(3-fluoro-4-hydroxy-phenyl)-6-hydroxy-3H-inden-1-yl]-thiophene-3-carbonitrile (E95);
2-[4-Fluoro-6-hydroxy-2-(4-hydroxy-phenyl)-3H-inden-1-yl]-furan-3-carbonitrile (E96);
3-[6-Hydroxy-2-(4-hydroxy-phenyl)-4-methyl-3H-inden-1-yl]-furan-2-carbonitrile (E97);
3-[2-(3-Fluoro-4-hydroxy-phenyl)-6-hydroxy-4-methyl-3H-inden-1-yl]-furan-2-carbonitrile (E98);
3-[2-(2,3-Difluoro-4-hydroxy-phenyl)-6-hydroxy-4-methyl-3H-inden-1-yl]-furan-2-carbonitrile (E99);
5-[6-Hydroxy-2-(4-hydroxy-phenyl)-4-trifluoromethyl-3H-inden-1-yl]-furan-2-carbonitrile (E100);
5-[2-(3-Fluoro-4-hydroxy-phenyl)-6-hydroxy-4-trifluoromethyl-3H-inden-1-yl]-furan-2-carbonitrile (E10);
2-[6-Hydroxy-2-(4-hydroxy-phenyl)-4-trifluoromethyl-3H-inden-1-yl]-furan-3-carbonitrile (E102);
2-[2-(3-Fluoro-4-hydroxy-phenyl)-6-hydroxy-4-trifluoromethyl-3H-inden-1-yl]-furan-3-carbonitrile (E103);
3-[6-Hydroxy-2-(4-hydroxy-phenyl)-4-trifluoromethyl-3H-inden-1-yl]-furan-2-carbonitrile (E104);
3-[2-(3-Fluoro-4-hydroxy-phenyl)-6-hydroxy-4-trifluoromethyl-3H-inden-1-yl]-furan-2-carbonitrile (E105);
3-[6-Hydroxy-2-(4-hydroxy-phenyl)-4-methyl-3H-inden-1-yl]-furan-2-carbaldehyde (E106);
4-[2-(2,4-Dihydroxy-phenyl)-6-hydroxy-4-methyl-3H-inden-1-yl]-thiophene-3-carbonitrile (E107);
7-Chloro-2-(4-hydroxy-phenyl)-3-phenyl-1H-inden-5-ol (E108);
4-[4-Chloro-2-(3-fluoro-4-hydroxy-phenyl)-6-hydroxy-3H-inden-1-yl]-thiophene-3-carbonitrile (E109);
2-[4-Chloro-6-hydroxy-2-(4-hydroxy-phenyl)-3H-inden-1-yl]-furan-3-carbonitrile (E110);
2-[4-Chloro-2-(3-fluoro-4-hydroxy-phenyl)-6-hydroxy-3H-inden-1-yl]-furan-3-carbonitrile (E111);
2-[2-(2,3-Difluoro-4-hydroxy-phenyl)-6-hydroxy-4-methyl-3H-inden-1-yl]-furan-3-carbonitrile (E112);
2-[2-(2,3-Difluoro-4-hydroxy-phenyl)-7-fluoro-6-hydroxy-3H-inden-1-yl]-furan-3-carbonitrile (E113);
2-[2-(3-Fluoro-4-hydroxy-phenyl)-7-fluoro-6-hydroxy-3H-inden-1-yl]-furan-3-carbonitrile (E114);
2-[2-(3-chloro-5-fluoro-4-hydroxy-phenyl)-7-fluoro-6-hydroxy-3H-inden-1-yl]-furan-3-carbonitrile (E115);
2-[2-(2,3-Difluoro-4-hydroxy-phenyl)-4-fluoro-6-hydroxy-3H-inden-1-yl]-furan-3-carbonitrile (E116);
2-[6-Hydroxy-2-(4-hydroxy-phenyl)-4-methyl-3H-inden-1-yl]-1-methyl-1H-imidazole-4-carbonitrile (E117);
2-[4-Chloro-2-(2,3-difluoro-4-hydroxy-phenyl)-6-hydroxy-3H-inden-1-yl]-furan-3-carbonitrile (E118);
2-[2-(2,3-Difluoro-4-hydroxy-phenyl)-6-hydroxy-4-trifluoromethyl-3H-inden-1-yl]-furan-3-carbonitrile (E119);
2-[6-Hydroxy-2-(4-hydroxy-phenyl)-3,4-dimethyl-3H-inden-1-yl]-furan-3-carbonitrile (E120);
2-[2-(3-Chloro-5-fluoro-4-hydroxy-phenyl)-6-hydroxy-4-methyl-3H-inden-1-yl]-furan-3-carbonitrile (E121);
2-[6-Methoxy-2-(4-methoxy-phenyl)-4-methyl-3H-inden-1-yl]-furan-3-carbonitrile (E122);
2-[7-Fluoro-6-hydroxy-2-(4-hydroxy-phenyl)-3H-inden-1-yl]-furan-3-carbonitrile (E123);
2-[4-Chloro-2-(3-chloro-5-fluoro-4-hydroxy-phenyl)-6-hydroxy-3H-inden-1-yl]-furan-3-carbonitrile (E124);
2-[4-Fluoro-2-(3-fluoro-4-hydroxy-phenyl)-6-hydroxy-3H-inden-1-yl]-furan-3-carbonitrile (E125);
2-[2-(3-Chloro-5-fluoro-4-hydroxy-phenyl)-4-fluoro-6-hydroxy-3H-inden-1-yl]-furan-3-carbonitrile (E126);
2-[2-(3-Chloro-5-fluoro-4-hydroxy-phenyl)-6-hydroxy-4-trifluoromethyl-3H-inden-1-yl]-furan-3-carbonitrile (E127);

The compound names given above were generated in accordance with IUPAC by the ACD Labs 8.0/name program, version 8.05 and/or with ISIS DRAW Autonom 2000.

Salts and solvates of compounds of formula (I) which are suitable for use in medicine are those wherein a counterion or associated solvent is pharmaceutically acceptable. However, salts and solvates having non-pharmaceutically acceptable counterions or associated solvents are within the scope of the present invention, for example, for use as intermediates in the preparation of the compounds of formula (I) and their pharmaceutically acceptable salts, solvates and physiologically functional derivatives. By the term “physiologically functional derivative” is meant a chemical derivative of a compound of formula (I) having the same physiological function as the free compound of formula (I), for example, by being convertible in the body thereto. According to the present invention, examples of physiologically functional derivatives include esters, amides, and carbamates; preferably esters and amides.

Suitable salts according to the invention include those formed with organic or inorganic acids or bases. In particular, suitable salts formed with acids according to the invention include those formed with mineral acids, strong organic carboxylic acids, such as alkanecarboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C₁-C₄)-alkyl- or aryl-sulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine. Other acids such as oxalic, while not in themselves pharmaceutically acceptable, may be useful as intermediates in obtaining the compounds of the invention and their pharmaceutical acceptable acid addition salts. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine. Corresponding internal salts may furthermore be formed.

Pharmaceutically acceptable esters and amides of the compounds of formula (I) may have an appropriate group, for example an acid group, converted to a C₁₋₆ alkyl, C₅₋₁₀ aryl, C₅₋₁₀ aryl-C₁₋₆ alkyl, or amino acid ester or amide. Pharmaceutically acceptable esters of the compounds of formula (I) may have an appropriate group, for example a hydroxy group, converted to a C₁₋₆ alkyl, C₅₋₁₀ aryl, or C₅₋₁₀ aryl-C₁₋₆ alkyl ester. Pharmaceutically acceptable amides and carbamates of the compounds of formula (I) may have an appropriate group, for example an amino group, converted to a C₁₋₆ alkyl, C₅₋₁₀ aryl, C₅₋₁₀ aryl-C₁₋₆ alkyl, or amino acid ester or amide, or carbamate.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”.

A compound which, upon administration to the recipient, is capable of being converted into a compound of formula (I) as described above, or an active metabolite or residue thereof, is known as a “prodrug”. A prodrug may, for example, be converted within the body, e.g. by hydrolysis in the blood, into its active form that has medical effects. Pharmaceutical acceptable prodrugs are described in T. Higuchi and V. Stella, Prodrugs as Novel Delivery Systems, Vol. 14 of the A. C. S. Symposium Series (1976); “Design of Prodrugs” ed. H. Bundgaard, Elsevier, 1985; and in Edward B. Roche, ed., Bioreversible Carriers in Drug Design, American Pharmaceutical Association and Pergamon Press, 1987, which are incorporated herein by reference.

The following definitions apply to the terms as used throughout this specification, unless otherwise limited in specific instances.

As used herein, the term “alkyl” means both straight and branched chain saturated hydrocarbon groups. Examples of alkyl groups include methyl, ethyl, n-propyl, iso-propyl, n-butyl, t-butyl, i-butyl, sec-butyl, pentyl, hexyl, heptyl, octyl, nonyl, and decyl groups. Among unbranched alkyl groups, there are preferred methyl, ethyl, n-propyl, iso-propyl, n-butyl groups. Among branched alkyl groups, there may be mentioned t-butyl, i-butyl, 1-ethylpropyl, 1-ethylbutyl, and 1-ethylpentyl groups.

As used herein, the term “alkoxy” means the group O-alkyl, where “alkyl” is used as described above. Examples of alkoxy groups include methoxy and ethoxy groups. Other examples include propoxy and butoxy.

As used herein, the term “alkenyl” means both straight and branched chain unsaturated hydrocarbon groups with at least one carbon carbon double bond. Up to 5 carbon carbon double bonds may, for example, be present. Examples of alkenyl groups include ethenyl, propenyl, butenyl, pentenyl, hexenyl, heptenyl, octenyl, nonenyl, decenyl and dodecenyl. Preferred alkynyl groups include ethenyl, 1-propenyl and 2-propenyl.

As used herein, the term “alkynyl” means both straight and branched chain unsaturated hydrocarbon groups with at least one carbon carbon triple bond. Up to 5 carbon carbon triple bonds may, for example, be present. Examples of alkynyl groups include ethynyl, propynyl, butynyl, pentynyl, hexynyl, heptynyl, octynyl, nonynyl, decynyl and dodecynyl. Preferred alkenyl groups include ethynyl 1-propynyl and 2-propynyl.

As used herein, the term “cycloalkyl” means a saturated group in a ring system. The cycloalkyl group can be monocyclic or bicyclic. A bicyclic group may, for example, be fused or bridged. Examples of monocyclic cycloalkyl groups include cyclopropyl, cyclobutyl and cyclopentyl. Other examples of monocyclic cycloalkyl groups are cyclohexyl, cycloheptyl and cyclooctyl. Examples of bicyclic cycloalkyl groups include bicyclo [2.2.1]hept-2-yl. Preferably, the cycloalkyl group is monocyclic.

As used herein, the term “aryl” means a monocyclic or bicyclic aromatic carbocyclic group. Examples of aryl groups include phenyl and naphthyl. A naphthyl group may be attached through the 1 or the 2 position. In a bicyclic aromatic group, one of the rings may, for example, be partially saturated. Examples of such groups include indanyl and tetrahydronaphthyl. Specifically, the term C₅₋₁₀ aryl is used herein to mean a group comprising from 5 to 10 carbon atoms in a monocyclic or bicyclic aromatic group. A particularly preferred C₅₋₁₀ aryl group is phenyl.

As used herein, the term “halogen” means fluorine, chlorine, bromine or iodine. Fluorine, chlorine and bromine are particularly preferred. In some embodiments, fluorine is especially preferred. In alternative embodiments, chlorine or bromine are especially preferred.

As used herein, the term “haloalkyl” means an alkyl group having a halogen substituent, the terms “alkyl” and “halogen” being understood to have the meanings outlined above. Similarly, the term “dihaloalkyl” means an alkyl group having two halogen substituents and the term “trihaloalkyl” means an alkyl group having three halogen substituents. Examples of haloalkyl groups include fluoromethyl, chloromethyl, bromomethyl, fluoromethyl, fluoropropyl and fluorobutyl groups; examples of dihaloalkyl groups include difluoromethyl and difluoroethyl groups; examples of triihaloalkyl groups include trifluoromethyl and trifluoroethyl groups.

As used herein, the term “heterocyclyl” means an aromatic (“heteroaryl”) or a non-aromatic (“heterocycloalkyl”) cyclic group of carbon atoms wherein from one to three of the carbon atoms is/are replaced by one or more heteroatoms independently selected from nitrogen, oxygen or sulfur. A heterocyclyl group may, for example, be monocyclic or bicyclic. In a bicyclic heterocyclyl group there may be one or more heteroatoms in each ring, or only in one of the rings. A heteroatom is preferably O or N. Heterocyclyl groups containing a suitable nitrogen atom include the corresponding N-oxides. Examples of monocyclic heterocycloalkyl rings include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl.

Examples of bicyclic heterocyclic rings in which one of the rings is non-aromatic include dihydrobenzofuranyl, indanyl, indolinyl, isoindolinyl, tetrahydroisoquinolinyl, tetrahydroquinolyl and benzoazepanyl.

Examples of monocyclic heteroaryl groups include furanyl, thienyl, pyrrolyl, oxazolyl, thiazolyl, imidazolyl, oxadiazolyl, thiadiazolyl, pyridyl, triazolyl, triazinyl, pyridazyl, pyrimidinyl, isothiazolyl, isoxazolyl, pyrazinyl, pyrazolyl and pyrimidinyl; examples of bicyclic heteroaryl groups include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridiyl, pyridopyrimidinyl, isoquinolinyl and benzodroxazole.

Examples of preferred heterocyclyl groups include piperidinyl, tetrahydrofuranyl, tetrahydropyranyl, pyridyl, pyrimidyl and indolyl. Preferred heterocyclyl groups also include thiophenyl, thiazolyl, furanyl, pyrazolyl, pyrrolyl and imidazolyl.

As used herein the term “cycloalkylalkyl” means a group cycloalkyl-alkyl-attached through the alkyl group, “cycloalkyl” and “alkyl” being understood to have the meanings outlined above.

As mentioned above, the compounds of the invention have activity as estrogen receptor ligands. The compounds of the invention have activity as estrogen receptor modulators, and may be agonists, partial agonists, antagonists, or partial antagonists of the estrogen receptor. Particularly preferred compounds of the invention have activity as an agonist or a partial agonist of ERβ. Preferred compounds of this type are selective agonists of the estrogen receptor-beta (ERβ).

The compounds of the invention may thus be used in the treatment of diseases or disorders associated with estrogen receptor activity. In particular, the compounds of the invention that are agonists or partial agonists of the estrogen receptor may be used in the treatment of diseases or disorders for which selective agonists or partial agonists of the estrogen receptor are indicated. The compounds of the invention that are antagonists or partial antagonists of the estrogen receptor may be used in the treatment of diseases or disorders for which selective antagonists or partial antagonists of the estrogen receptor are indicated.

Clinical conditions for which an agonist or partial agonist is indicated include, but are not limited to, bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression, autoimmune disease, inflammation, IBD, IBS, sexual dysfunction, hypertension, retinal degeneration, and lung, colon, breast, uterus, and prostate cancer, and/or disorders related to estrogen functioning.

The compounds of the invention find particular application in the treatment or prophylaxis of the following: bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression, autoimmune disease, inflammation, IBD, IBS, sexual dysfunction, hypertension, retinal degeneration, and lung, colon, breast, uterus, and prostate cancer, and/or disorders related to estrogen functioning.

The invention also provides a method for the treatment or prophylaxis of a condition in a mammal mediated by an estrogen receptor, which comprises administering to the mammal a therapeutically effective amount of a compound of formula (I) as defined above or a pharmaceutically acceptable ester, amide, solvate or salt thereof, including a salt of such an ester or amide, and a solvate of such an ester, amide or salt. Clinical conditions mediated by an estrogen receptor that may be treated by the method of the invention are those described above.

The invention also provides the use of a compound of formula (I) as defined above or a pharmaceutically acceptable ester, amide, solvate or salt thereof, including a salt of such an ester or amide, and a solvate of such an ester, amide or salt, for the manufacture of a medicament for the treatment or prophylaxis of a condition mediated by an estrogen receptor. Clinical conditions mediated by an estrogen receptor that may be treated by the method of the invention are those described above.

Hereinafter, the term “active ingredient” means a compound of formula (I) as defined above, or a pharmaceutically acceptable ester, amide, solvate or salt thereof, including a salt of such an ester or amide, and a solvate of such an ester, amide or salt.

The amount of active ingredient which is required to achieve a therapeutic effect will, of course, vary with the particular compound, the route of administration, the subject under treatment, including the type, species, age, weight, sex, and medical condition of the subject and the renal and hepatic function of the subject, and the particular disorder or disease being treated, as well as its severity. An ordinarily skilled physician, veterinarian or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, preferably 0.01 mg per kg of body weight per day (mg/kg/day) to 10 mg/kg/day, and most preferably 0.1 to 5.0 mg/kg/day, for adult humans. For oral administration, the compositions are preferably provided in the form of tablets or other forms of presentation provided in discrete units containing 0.01, 0.05, 0.1, 0.5, 1.0, 2.5, 5.0, 10.0, 15.0, 25.0, 50.0, 100, and 500 milligrams of the active ingredient for the symptomatic adjustment of the dosage to the patient to be treated. A medicament typically contains from about 0.01 mg to about 500 mg of the active ingredient, preferably from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

While it is possible for the active ingredient to be administered alone, it is preferable for it to be present in a pharmaceutical formulation or composition. Accordingly, the invention provides a pharmaceutical formulation comprising a compound of formula (I) as defined above or a pharmaceutically acceptable ester, amide, solvate or salt thereof, including a salt of such an ester or amide, and a solvate of such an ester, amide or salt, and a pharmaceutically acceptable diluent, excipient or carrier (collectively referred to herein as “carrier” materials). Pharmaceutical compositions of the invention may take the form of a pharmaceutical formulation as described below.

The pharmaceutical formulations according to the invention include those suitable for oral, parenteral (including subcutaneous, intradermal, intramuscular, intravenous [bolus or infusion], and intraarticular), inhalation (including fine particle dusts or mists which may be generated by means of various types of metered does pressurized aerosols), nebulizers or insufflators, rectal, intraperitoneal and topical (including dermal, buccal, sublingual, and intraocular) administration, although the most suitable route may depend upon, for example, the condition and disorder of the recipient.

The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets, pills or tablets each containing a predetermined amount of the active ingredient; as a powder or granules; as a solution or a suspension in an aqueous liquid or a non-aqueous liquid, for example as elixirs, tinctures, suspensions or syrups; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The present compounds can, for example, be administered in a form suitable for immediate release or extended release. Immediate release or extended release can be achieved by the use of suitable pharmaceutical compositions comprising the present compounds, or, particularly in the case of extended release, by the use of devices such as subcutaneous implants or osmotic pumps. The present compounds can also be administered liposomally.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate, calcium sulfate, sorbitol, glucose and/or lactose and/or other excipients, binders, extenders, disintegrants, diluents and lubricants such as those known in the art. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Disintegrators include without limitation starch, methylcellulose, agar, bentonite, xanthan gum and the like. The compounds of formula (I) can also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use. Lubricants used in these dosage forms include sodium oleate, sodium stearate, magnesium stearate, sodium benzoate, sodium acetate, sodium chloride and the like. For oral administration in liquid form, the oral drug components can be combined with any oral, non-toxic, pharmaceutically acceptable inert carrier such as ethanol, glycerol, water, and the like.

The compounds of the present invention can also be administered in the form of liposome delivery systems, such as small unilamellar vesicles, large unilamellar vesicles and multilamellar vesicles. Liposomes can be formed from a variety of phospholipids, 1,2-dipalmitoylphosphatidylcholine, phosphatidyl ethanolamine (cephaline), or phosphatidylcholine (lecithin).

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Exemplary compositions for nasal, aerosol or inhalation administration include solutions in saline, which can contain, for example, benzyl alcohol or other suitable preservatives, absorption promoters to enhance bioavailability, and/or other solubilizing or dispersing agents such as those known in the art.

Formulations for rectal administration may be presented as a suppository with the usual carriers such as cocoa butter, synthetic glyceride esters or polyethylene glycol. Such carriers are typically solid at ordinary temperatures, but liquefy and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

Whilst a compound of the invention may be used as the sole active ingredient in a medicament, it is also possible for the compound to be used in combination with one or more further active agents. Such further active agents may be further compounds according to the invention, or they may be different therapeutic agents, for example an antidepressant, an anxiolytic, an anti-psychotic, or an agent useful in the prevention or treatment of osteoporosis or other pharmaceutically active material. For example, the compounds of the instant invention may be effectively administered in combination with effective amounts of other agents such as an antidepressant, an anxiolytic, an anti-psychotic, an organic bisphosphonate or a cathepsin K inhibitor. Nonlimiting examples of antidepressants include noradrenaline reuptake inhibitors (NRI), selective serotonin reuptake inhibitors, monoamine oxidase inhibitors, tricyclic antidepressants (TCA), dopamine reuptake inhibitors (DRI), opioids, selective seretonic reuptake enhancers, tetracyclic antidepressants, reversible inhibitors of monoamine oxidase, melatonin agonists, serotonin and noradrenaline reuptake inhibitors (SNRI), corticotropin releasing factor antagonists, α-adrenoreceptor antagonists, 5HT1α receptor agonists and antagonists, lithium and atypical anti-psychotics. Examples of antidepressants of the SSRI class include Fluoxetine and Sertraline; examples of antidepressants of the SNRI class Venlafaxine, Citalopram, Paroxetine, Escitalopram, Fluvoxamine; examples of antidepressants of the SNRI class include Duloxetine; examples of antidepressants of the DRI and NRI classes include Bupropion; examples of antidepressants of the TCA class include Amitriptyline and Dothiepin (Dosulepin). Examples of atypical antipsychotics include: Clozapine, Olanzapine, Risperidone, Quetiapine, Ziprasidone and Dopamine partial agonists. Nonlimiting examples of anxiolytics include benzodiazepines and non-benzodiazapines. Examples of benzodiazapines include lorazepam, alprazolam, and diazepam. Examples of non-benzodiazapines include Buspirone (Buspar®), barbiturates and meprobamate. One or more of those further anti-depressants may be used in combination.

Nonlimiting examples of said organic bisphosphonates include adendronate, clodronate, etidronate, ibandronate, incadronate, minodronate, neridronate, risedronate, piridronate, pamidronate, tiludronate, zoledronate, pharmaceutically acceptable salts or esters thereof, and mixtures thereof. Preferred organic biphosphonates include alendronate and pharmaceutically acceptable salts and mixtures thereof. Most preferred is alendronate monosodium trihydrate.

The precise dosage of the bisphosphonate will vary with the dosing schedule, the oral potency of the particular bisphosphonate chosen, the age; size, sex and condition of the mammal or human, the nature and severity of the disorder to be treated, and other relevant medical and physical factors. Thus, a precise pharmaceutically effective amount cannot be specified in advance and can be readily determined by the caregiver or clinician. An appropriate amount can be determined by routine experimentation from animal models and human clinical studies. Generally, an appropriate amount of bisphosphonate is chosen to obtain a bone resorption inhibiting effect, i.e. a bone resorption inhibiting amount of the bisphonsphonate is administered. For humans, an effective oral dose of bisphosphonate is typically from about 1.5 to about 6000 μg/kg of body weight and preferably about 10 to about 2000 μg/kg of body weight.

For human oral compositions comprising alendronate, pharmaceutically acceptable salts thereof, or pharmaceutically acceptable derivatives thereof, a unit dosage typically comprises from about 8.75 mg to about 140 mg of the alendronate compound, on an alendronic acid active weight basis, i.e. on the basis of the corresponding acid.

The compounds of the present invention can be used in combination with other agents useful for treating estrogen-mediated conditions. The individual components of such combinations can be administered separately at different times during the course of therapy or concurrently in divided or single combination forms. The present invention is therefore to be understood as embracing all such regimes of simultaneous or alternating treatment and the term “administering” is to be interpreted accordingly. It will be understood that the scope of combinations of the compounds of this invention with other agents useful for treating estrogen-mediated conditions includes in principle any combination with any pharmaceutical composition useful for treating disorders related to estrogen functioning.

The above other therapeutic agents, when employed in combination with the compounds of the present invention, may be used, for example, in those amounts indicated in the Physicians' Desk Reference (PDR) or as otherwise determined by one of ordinary skill in the art.

Where the compounds of the invention are utilized in combination with one or more other therapeutic agent(s), either concurrently or sequentially, the following combination ratios and dosage ranges are preferred:

When combined with an antidepressant, an anxiolytic, an anti-psychotic, an organic bisphosphonate or a cathepsin K inhibitor, the compounds of formula (I) may be employed in a weight ratio to the additional agent within the range from about 10:1 to about 1:10.

The compounds of formula (I) as described above also find use, optionally in labelled form, as a diagnostic agent for the diagnosis of conditions associated with malfunction of the estrogen receptor. For example, such a compound may be radioactively labelled.

The compounds of formula (I) as described above, optionally in labelled form, also find use as a reference compound in methods of discovering other agonists, partial agonists, antagonists or partial antagonists of the estrogen receptor. Thus, the invention provides a method of discovering a ligand of the estrogen receptor which comprises use of a compound of the invention or a compound of the invention in labelled form, as a reference compound. For example, such a method may involve a competitive binding experiment in which binding of a compound of formula (I) to the estrogen receptor is reduced by the presence of a further compound which has estrogen receptor-binding characteristics, for example stronger estrogen receptor-binding characteristics than the compound of formula (I) in question.

Numerous synthetic routes to the compounds of the present invention can be devised by any person skilled in the art and the possible synthetic routes described below do not limit the invention. Many methods exist in the literature for the synthesis of indenes, for example: Organometallics, 25, 1217-1229, 2006, Org. Lett., 7, 4963-4966, 2005, J. Am. Chem. Soc., 127, 13498-13499, 2005, J. Org. Chem., 58, 4579-4583, 1993, J. Org. Chem., 54, 1485-1491, 1989.

Accordingly, the invention provides a method for preparing a compound of formula (I) in accordance with the invention as described above wherein the bond between the C1 and C2 carbon atoms is a double bond, and R¹ and R² are both hydrogen, comprising a step of reacting a compound of formula (II)

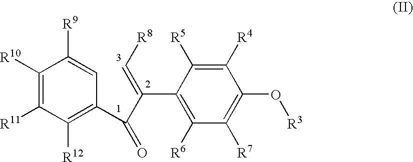

wherein R³, R⁴, R⁵, R⁶, R⁷, R⁸, R⁹, R¹⁰, R¹¹ and R¹² are as defined above
with a suitable reducing agent, and optionally followed by interconversion to another compound of formula (I) in accordance with the invention as described above.

Suitable reducing agents include sodium bis(2-methoxyethoxy)aluminium hydride. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

The invention also provides a method for preparing a compound of formula (I) in accordance with the invention as described above wherein the bond between the C1 and C2 carbon atoms is a double bond, comprising a step of reacting a compound of formula (III)

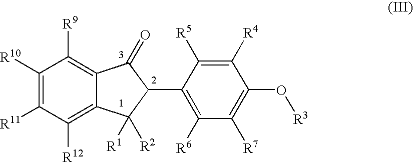

wherein R¹, R², R³, R⁴, R⁵, R⁶, R⁷, R⁹, R¹⁰, R¹¹ and R¹² are as defined above
with a compound of formula (IV)

R⁸-M (IV)

wherein R⁸ is as defined above and M is a suitable metal, for example lithium, magnesium bromide or magnesium chloride, and optionally followed by interconversion to another compound of formula (I) in accordance with the invention as described above.

The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

The invention also provides a method for preparing a compound of formula (I) in accordance with the invention as described above wherein the bond between the C2 and C3 carbon atoms is a double bond and R² is hydrogen, comprising a step of reacting a compound of formula (V)

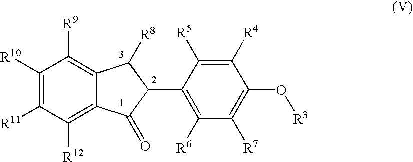

wherein R³, R⁴, R⁵, R⁶, R⁷, R⁸, R⁹, R¹⁰, R¹¹ and R¹² are as defined above
with a compound of formula (VI)

R⁸-M (VI)

wherein R⁸ is as defined above and M is a suitable metal, for example lithium, magnesium bromide or magnesium chloride, and optionally followed by interconversion to another compound of formula (I) in accordance with the invention as described above.

The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

### EXAMPLES

The novel compounds of the present invention can be prepared according to the procedure of the following Schemes and examples, using appropriate materials and are further exemplified by the following specific examples. The compounds illustrated in the examples are not, however, to be construed as forming the only genus that is considered as the invention. The following examples further illustrate details for the preparation of the compounds of the present invention. Those skilled in the art will readily understand that known variation of the conditions and processes of the following preparative procedures can be used to prepare these compounds.

### General Experimental Conditions

The compounds of the present invention of formula (I) are prepared according to the general methods outlined in Schemes 1-7, and according to the related methods described. All temperatures are degrees Celsius unless otherwise noted. The following abbreviations, reagents, expressions or equipment, which are amongst those used in the descriptions below, are explained as follows: 20-25° C. (room temperature, r.t.), molar equivalent (eq.), dimethyl formamide, (DMF) dichloromethane (DCM), ethyl acetate (EtOAc), tetrahydrofuran (THF), lithium diisopropylamide (LDA), pyridinium chlorochromate (PCC), preparative liquid chromatography with a C8 stationary phase and ammonium acetate acetonitrile-water buffer as mobile phase (PHPLC), electrospray mass spectroscopy (ES/MS).

### Example 1

### 2-(4-Hydroxy-phenyl)-7-methyl-3-p-tolyl-1H-inden-5-ol (E1)

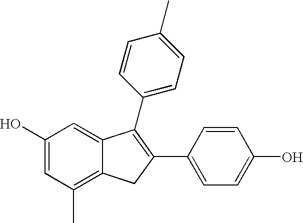

The title compound was synthesized according to the method outlined in Scheme 1.

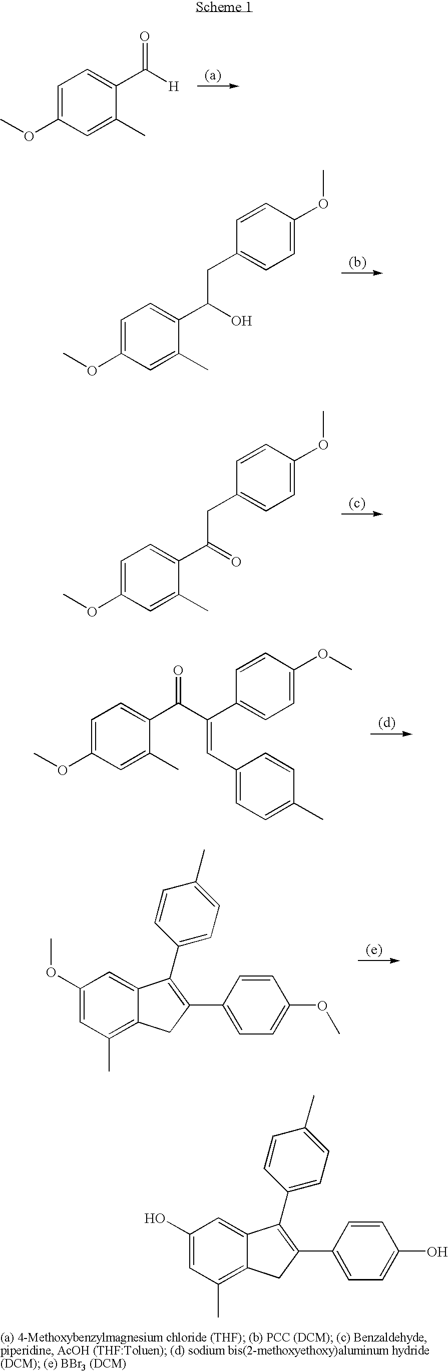

Step (a): 1.1 eq 4-Methoxybenzylmagensium chloride was stirred under an N₂-atmosphere and 1 eq 4-Methoxy-2-methyl-benzaldehyde was added drop wise over 15 min. The reaction was stirred at r.t. overnight and then quenched with the addition of H₂0. The reaction mixture was diluted with EtOAc. The organic phase was washed several times with Brine. The phases were separated and the organic phase was dried over MgSO₄ and then filtered and evaporated in vacuo. The crude product was purified on silica column using 4:1 n-Heptane:EtOAc as mobile phase.

Step (b): 1-(4-Methoxy-2-methyl-phenyl)-2-(4-methoxy-phenyl)-ethanol was dissolved in DCM and 2.1 eq PCC were added. The reaction was stirred at r.t overnight. H₂O was added and then the reaction was diluted with DCM. The organic phase was washed with Brine several times and then the phases were separated on a SPE Phase Separator. The organic phase was evaporated in vacuo. The crude product was purified on a silica column with 6:1 n-Heptane:EtOAc as mobile phase. Product obtained as a yellow solid.

Step (c): 1-(4-Methoxy-2-methyl-phenyl)-2-(4-methoxy-phenyl)-ethanone was dissolved in 1:9 THF:Toluene (dry solvents) mixture. 0.2 eq Piperidine and 1 eq AcOH were added and then the reaction was heated to 110° C. 4 eq Benzaldehyde were added and the reaction was stirred at 110° C. overnight. Brine was added and the reaction was then diluted with DCM. The organic phase was washed with NaHCO₃ (aq) several times and then the phases were separated on a SPE Phase Separator. The organic phase was evaporated in vacuo and the resulting crude product was purified on Argonaut Flashmaster MPLC with n-Heptane:EtOAc gradient mobile phase (1% EtOAc to 99% EtOAc over 30 min).

Step (d): (E)-1-(4-Methoxy-2-methyl-phenyl)-2-(4-methoxy-phenyl)-3-phenyl-propenone was dissolved in DCM and 2 eq “Red-Al” (sodium bis(2-methoxyethoxy)aluminum hydride) were added and the reaction was stirred for 6 hours at r.t. The reaction was then quenched with the addition of brine and the crude reaction was diluted with DCM. The organic phase was washed with NH₄Cl(aq) several times and then the phases were separated on a SPE Phase Separator. The organic phase was concentrated and the crude product was used directly in next step without purification.

Step (e): Crude 5-Methoxy-2-(4-methoxy-phenyl)-7-methyl-3-phenyl-1H-indene was dissolved in dry DCM and cooled to 0° C. 2.3 eq BBr₃-solution 1.0M in hexane were added and the reaction was stirred at r.t. for 2 h. The reaction was then quenched with EtOH, diluted with DCM and then washed with NaHCO₃(aq) several times before phases were separated on a SPE Phase Separator. The organic phase was concentrated and the crude product was purified on a silica column using 3:1 n-Heptane:EtOAc as mobile phase. ES/MS m/z: 329 (pos. M+H), 327 (neg. M−H); ¹H NMR (500 MHz, Acetone-d₆) δ 8.36 (s, 1H), 7.84 (s, 1H) 7.28−7.26 (d, 2H), 7.20−7.19 (d, 4H), 6.70−6.68 (d, 2H), 6.52 (s, 1H), 6.43 (s, 1H), 3.69 (s, 2H), 2.39 (s, 3H), 2.33 (s, 3H).

### Example 2

### 3-(4-Fluoro-phenyl)-2-(4-hydroxy-phenyl)-7-methyl-1H-inden-5-ol (E2)

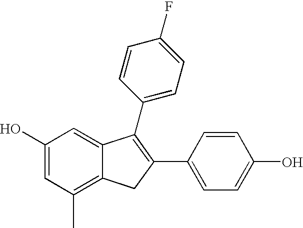

The title compound was synthesized using the procedures described in example 1. ES/MS m/z: 334 (pos. M+2H), 331 (neg., M−H); ¹H NMR (500 MHz, Acetone-d₆) δ 8.40 (s, 1H), 7.90 (s, 1H) 7.35−7.33 (m, 2H), 7.24−7.21 (t, 2H), 7.18−7.16 (d, 2H), 6.72−6.70 (d, 2H), 6.54 (s, 1H), 6.43 (s, 1H), 3.71 (s, 2H), 2.33 (s, 3H).

### Example 3

### 3-(4-Hydroxy-Z-methyl-phenyl)-t-(4-hydroxy-phenyl)-7-methyl-1H-inden-5-ol (E3)

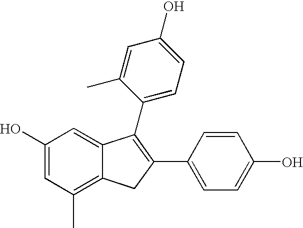

The title compound was synthesized using the procedures described in example 1. ES/MS m/z: 345 (pos. M+H), 343 (neg., M−H); ¹H NMR (500 MHz, CDCl₃) δ 7.22−7.20 (d, 2H), 6.98−6.96 (d, 1H), 6.83 (broad s, 1H), 6.80−6.77 (d, 1H), 6.69−6.67 (d, 2H), 6.49 (s, 1H), 6.16 (s, 1H), 3.75 (s, 1H), 3.71 (s, 1H), 2.33 (s, 3H), 1.96 (s, 3H).

### Example 4

### 2-(4-Hydroxy-phenyl)-3-phenyl-1H-inden-5-ol (E4)

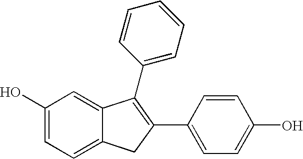

The title compound was synthesized according to the method outlined in Scheme 2.

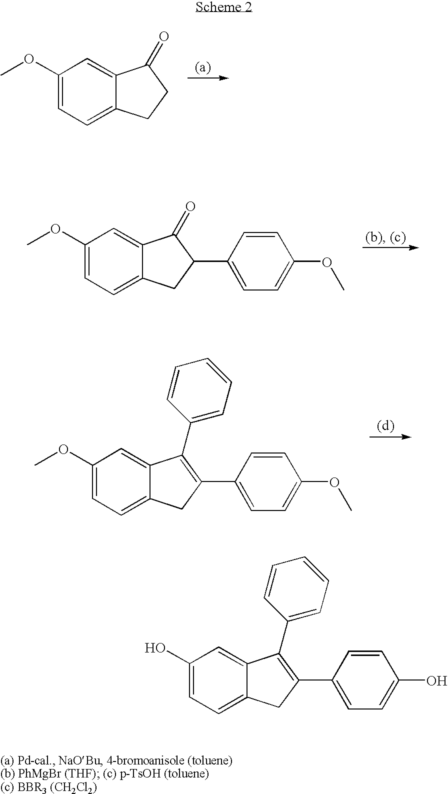

Step a: 1 mmol of 6-methoxyindanone, 1.5 mmol of 4-bromoanisole, 0.02 mmol of Pd(OAc)₂, 0.04 mmol of dicyclohexyl-(2′-methyl-biphenyl-2-yl)-phosphane, and 1.5 mmol NaO'Bu were heated to 80° C. for 5 h. 1M HCl and dichloromethane were added and the phases were separated. After flash chromatographic separation 0.26 mmol of 6-methoxy-2-(4-methoxy-phenyl)-indan-1-one were obtained.

Step b and c: To 0.25 mmol of 6-methoxy-2-(4-methoxy-phenyl)-indan-1-one in 2 ml THF was added 1 mmol of PhMgBr at 0° C. After 30 min the mixture was warmed to room temperature and stirred for additional 30 min. 1M HCl and dichloromethane were added and the phases were separated. The crude mixture was filtered through silica and dissolved in 2 ml toluene. 5 mg p-TsOH were added and the mixture was heated to 80° C. for 1 h. After flash chromatographic separation 0.13 mmol of 5-methoxy-2-(4-methoxy-phenyl)-3-phenyl-1H-indene were obtained.

Step d: To 0.13 mmol of 5-methoxy-2-(4-methoxy-phenyl)-3-phenyl-1H-indene in 3 ml dichloromethane were added 1.3 mmol BBr₃ (1M solution in dichloromethane) at 0° C. The mixture was allowed to slowly warm to room temperature. After a total reaction time of 5 h 1M HCl and dichloromethane were added and the phases were separated. After flash chromatographic separation 0.10 mmol of 2-(4-hydroxy-phenyl)-3-phenyl-1H-inden-5-ol were obtained. ES/MS m/z: 281.6 (pos, M+H); 279.4 (neg, M−M); ¹H NMR (500 MHz, CDCl₃) δ 7.32 (d, 2H), 7.27 (d, 1H), 6.88 (d, 2H), 6.85 (d, 1H), 6.65 (dd, 1H), 3.61 (s, 2H), 2.61-2.67 (m, 2H), 1.60-1.67 (m, 2H), 1.40-1.47 (m, 2H), 0.95 (t, 3H).

### Example 5

### 2-(4-Hydroxy-phenyl)-3-thiophen-2-yl-1H-inden-5-ol (E5)

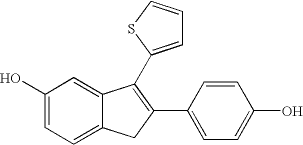

The title compound was synthesized using the methods described in example 4. ES/MS m/z: 307.4 (pos, M+H), 305.5 (neg, M−H); ¹H NMR (500 MHz, CDCl₃) δ 7.30 (dd, 1H), 7.26 (d, 1H), 7.17 (d, 2H), 7.05 (dd, 1H), 6.99 (dd, 1H), 6.78 (d, 1H), 6.62-6.68 (m, 3H), 3.73 (s, 2H).

### Example 6

### 7-Ethyl-2-(4-hydroxy-phenyl)-3-phenyl-1H-inden-5-ol (E6)

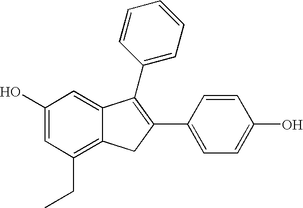

The title compound was synthesized according to the method outlined in Scheme 3.

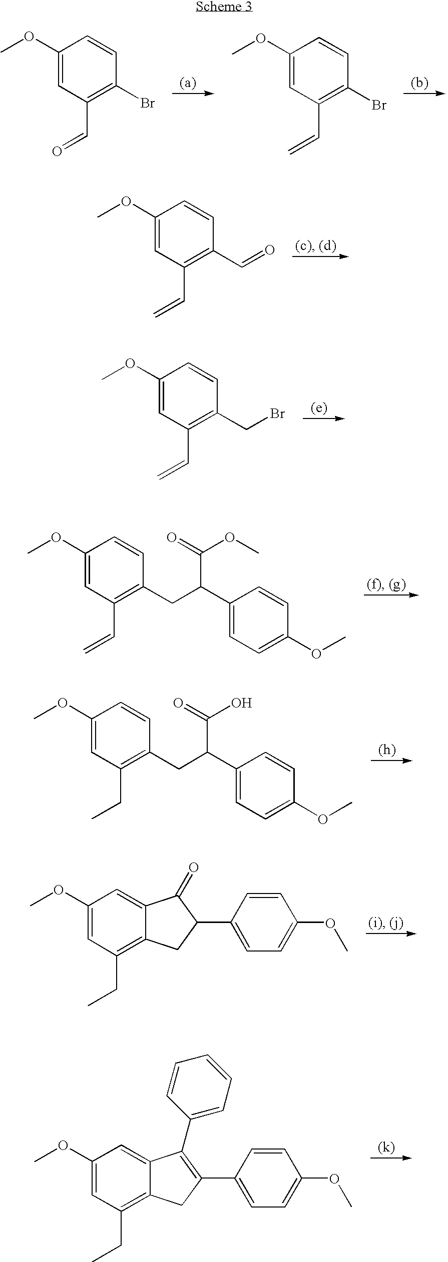

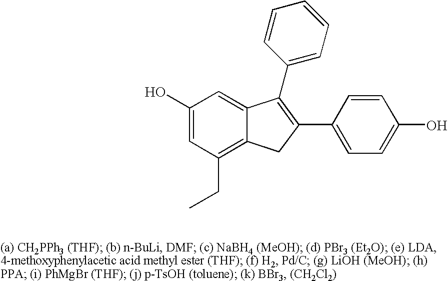

Step (a): 2 mmol of methyl-triphenylphosphonium bromide were suspended in 2 ml THF and cooled to 0° C. 1.8 mmol of n-BuLi were added dropwise. After 5 min 1 mmol of 2-bromo-5-methoxybenzaldehyde in 1 ml THF was added and the mixture was heated to 65° C. for 1 h. 1M HCl and dichloromethane were added and the phases were separated. After flash chromatographic separation 0.38 mmol of 1-bromo-4-methoxy-2-vinyl-benzene were obtained.

Step (b): 0.38 mmol of 1-bromo-4-methoxy-2-vinyl-benzene were dissolved in 3 ml THF and cooled to −78° C. 0.5 mmol of n-BuLi were slowly added and the mixture was stirred for 15 min. 1 mmol DMF was added and the cooling bath was removed. After 30 min 1M HCl and dichloromethane were added and the phases were separated. After flash chromatographic separation 0.185 mmol of 4-methoxy-2-vinyl-benzaldehyde were obtained.

Step (c), (d): 0.15 mmol of 4-methoxy-2-vinyl-benzaldehyde was dissolved in 1 ml MeOH and 0.1 mmol of NaBH₄ were added at 0° C. After 5 min the mixture was warmed to room temperature. After 15 min 1M HCl and dichloromethane were added and the phases were separated.

The crude mixture was dissolved in 1.5 ml diethylether and cooled to 0° C. 0.15 mmol of phosphorus tribromide were added and the mixture was stirred for 30 mint. The reaction mixture was filtered through silica to obtain 0.117 mmol of 1-bromomethyl-4-methoxy-2-vinyl-benzene.

Step (e): 0.8 mmol of 4-methoxy-phenylacetic acid methyl ester were dissolved in 4 ml THF and cooled to −78° C. 0.8 mmol of LDA were added and the mixture was stirred for 15 min. 1.0 mmol of 1-bromomethyl-4-methoxy-2-vinyl-benzene in 2 ml THF was added and the mixture was slowly warmed to room temperature. After a total reaction time of 2 h 1M HCl and dichloromethane were added and the phases were separated. After flash chromatographic separation 0.26 mmol of 2-(4-methoxy-phenyl)-3-(4-methoxy-2-vinyl-phenyl)-propionic acid methyl ester were obtained.

Step (f) and (g): 0.22 mmol of 2-(4-methoxy-phenyl)-3-(4-methoxy-2-vinyl-phenyl)-propionic acid methyl ester were dissolved in 1.5 ml EtOH and 1.5 ml EtOAc. 0.022 mmol Pd/C (10% w/w) were added and the mixture was hydrogenated under 0.5 bar of hydrogen at room temperature. After 2 h the mixture was filtered through silica. The crude material was dissolved in 2.5 ml MeOH and 2.2 mmol of LiOH were added. The mixture was warmed to 70° C. for 2 h. 1M HCl and dichloromethane were added and the phases were separated. 0.22 mmol of 3-(2-ethyl-4-methoxy-phenyl)-2-(4-methoxy-phenyl)-propionic acid was taken to the next step without further purification.

Step (h): 0.22 mmol of 3-(2-ethyl-4-methoxy-phenyl)-2-(4-methoxy-phenyl)-propionic acid were heated to 110° C. in ca. 5 ml polyphosphoric acid for 40 min. The mixture was cooled to room temperature, water was added and the mixture was extracted with EtOAc. After flash chromatographic separation 0.04 mmol of 4-ethyl-6-methoxy-2-(4-methoxy-phenyl)-indan-1-one were obtained.

Step (i), (j): To 0.04 mmol of 6-methoxy-2-(4-methoxy-phenyl)-indan-1-one in 1 ml THF were added 0.16 mmol of PhMgBr at 0° C. After 30 min the mixture was warmed to room temperature and stirred for additional 30 min. 1M HCl and dichloromethane were added and the phases were separated. The crude mixture was filtered through silica and dissolved in 2 ml toluene. 5 mg p-TsOH were added and the mixture was heated to 80° C. for 1 h. 0.033 mmol of 7-ethyl-5-methoxy-2-(4-methoxy-phenyl)-3-phenyl-1H-indene were obtained and used in the next step without further purification.

Step (k): To 0.033 mmol of 7-ethyl-5-methoxy-2-(4-methoxy-phenyl)-3-phenyl-1H-indene in 1 ml dichloromethane were added 0.33 mmol BBr₃ (1M solution in dichloromethane) at 0° C. The mixture was allowed to slowly warm to room temperature. After a total reaction time of 5 h 1M HCl and dichloromethane were added and the phases were separated. After flash chromatographic separation 0.014 mmol of 7-ethyl-2-(4-hydroxy-phenyl)-3-phenyl-1H-inden-5-ol were obtained. ES/MS m/z: 329.3 (pos, M+H), 327.4 (neg, M−H); ¹H NMR (500 MHz, CDCl₃) δ 7.38-7.44 (m, 2H), 7.31-7.38 (m, 3H), 7.17 (d, 2H), 6.67 (d, 2H), 6.58 (d, 1H), 6.50 (d, 1H), 3.74 (s, 2H), 2.73 (q, 2H), 1.32 (t, 3H).

### Example 7

### 2-(4-Hydroxy-phenyl)-3-phenyl-7-propyl-1H-inden-5-ol (E7)

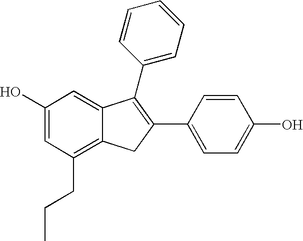

The title compound was synthesized using the procedures described in example 6. ES/MS m/z: 343.6 (pos, M+H), 341.2 (neg, M−H); ¹H NMR (500 MHz, CDCl₃) δ 7.38-7.44 (m, 2H), 7.30-7.37 (m, 3H), 7.17 (d, 2H), 6.67 (d, 2H), 6.55 (d, 1H), 6.50 (d, 1H), 3.74 (s, 2H), 2.67 (t, 2H), 1.74 (m, 2H), 1.40-1.47 (sext, 2H), 1.02 (t, 3H).

### Example 8

### 2-(4-Hydroxy-phenyl)-7-methyl-3-phenyl-1H-inden-5-ol (E8)

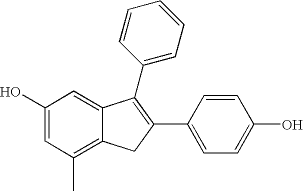

The title compound was synthesized according to the method outlined in Scheme 4.

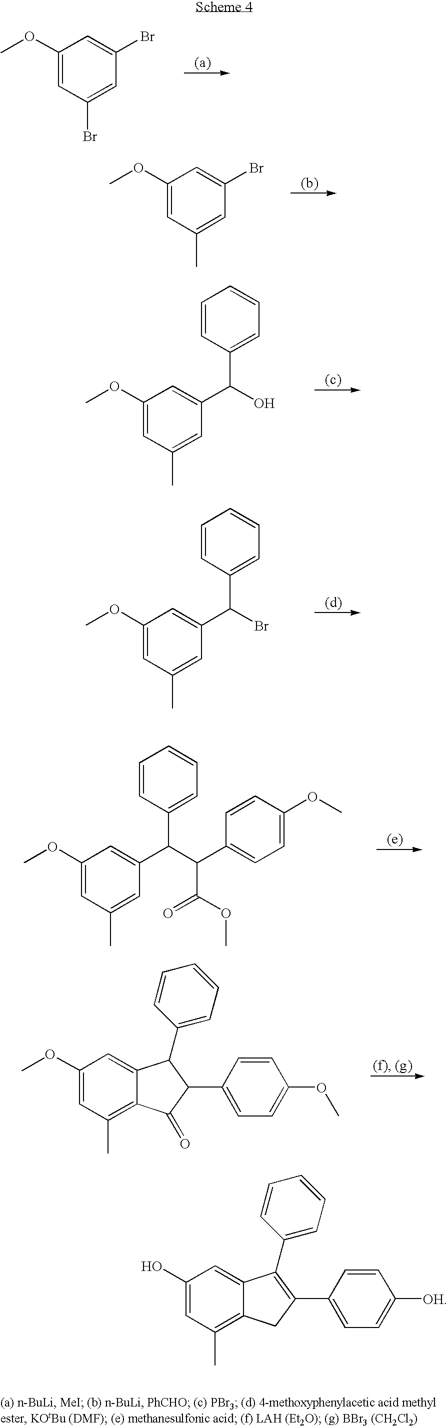

Step (a): 2 mmol 3,5-dibromoanisole were dissolved in 10 ml THF and cooled to −78° C. 2.4 mmol of n-BuLi were added dropwise and stirring was continued for 15 min. 3 mmol of methyl iodide were added and the mixture was allowed to slowly warm to ca. −20° C. over a period of ca. 2 h. 1M HCl and dichloromethane were added and the phases were separated. 1.65 mmol of crude 3-bromo-5-methylanisole were taken to the next step without further purification.

Step (b): 1 mmol 3-bromo-5-methylanisole were dissolved in 5 ml THF and cooled to −78° C. 1.2 mmol of n-BuLi were added dropwise and stirring was continued for IS min. 1.5 mmol of benzaldehyde were added and the mixture was allowed to slowly warm to ca. −20° C. over a period of ca. 2 h. 1M HCl and dichloromethane were added and the phases were separated. After flash chromatographic separation, 0.66 mmol of (3-methoxy-5-methyl-phenyl)-phenyl-methanol were obtained.

Step (c): 0.65 mmol of (3-methoxy-5-methyl-phenyl)-phenyl-methanol were dissolved in 6 ml diethylether and cooled to 0° C. 0.65 mmol of phosphorus tribromide were added and the mixture was stirred for 30 min. The reaction mixture was filtered through silica to obtain 0.57 mmol of 1-(bromo-phenyl-methyl)-3-methoxy-5-methyl-benzene.

Step (d): 1 mmol of 4-methoxy-phenylacetic acid methyl ester, 1.4 mmol of 1-(bromo-phenyl-methyl)-3-methoxy-5-methyl-benzene and 1.2 mmol of KO′Bu were stirred in 5 ml DMF at room temperature for 1.5 h. 1M HCl and dichloromethane were added and the phases were separated. After flash chromatographic separation, 0.84 mmol of 3-(3-methoxy-5-methyl-phenyl)-2-(4-methoxy-phenyl)-3-phenyl-propionic acid methyl ester were obtained as a mixture of diastereomers (50:50).

Step (e): 2 mmol of 3-(3-methoxy-5-methyl-phenyl)-2-(4-methoxy-phenyl)-3-phenyl-propionic acid methyl ester were heated to 90° C. in methanesulfonic acid for 90 min. The solution was poured into 1N NaOH solution and dichloromethane were added and the phases were separated. After flash chromatographic separation 1.0 mmol of 5-methoxy-2-(4-methoxy-phenyl)-7-methyl-3-phenyl-indan-1-one was obtained as a single diastereomer.

Step (f) and (g): 2 mmol of 5-methoxy-2-(4-methoxy-phenyl)-7-methyl-3-phenyl-indan-1-one were dissolved in 30 ml diethylether. 2 mmol of lithium aluminium hydride were added and after 10 min water and dichloromethane were slowly added. To the crude mixture in 20 ml dichloromethane were added 15 mmol BBr₃ (1M solution in dichloromethane) at 0° C. The mixture was allowed to slowly warm to room temperature. After a total reaction time of 2 h 1M HCl and dichloromethane were added and the phases were separated. After flash chromatographic separation 1.43 mmol of 2-(4-hydroxy-phenyl)-7-methyl-3-phenyl-1H-inden-5-ol were obtained. ES/MS m/z: 315.2 (pos, M+H), 313.3 (neg, M−H); ¹H NMR (500 MHz, d⁶-acetone) δ 8.42 (s, 1H, OH), 7.92 (s, 1H, OH), 7.48-7.55 (m, 2H), 7.40-7.46 (m, 1H), 7.35-7.39 (m, 2H), 7.24 (d, 2H), 6.74 (d, 2H), 6.59 (s, 1H), 6.48 (s, 1H), 3.76 (s, 2H), 2.39 (s, 3H).

### Example 9

### 2-(4-Hydroxy-phenyl)-1-phenyl-3H-indene-4,6-diol (E9)

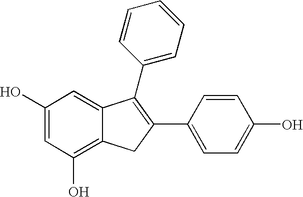

The title compound was synthesized using the procedures described in example 8. ES/MS m/z: 317.6 (pos, M+H), 315.7 (neg, M−H); ¹H-NMR (500 MHz, acetone-d₆) δ 7.46−7.42 (m, 2H), 7.38−7.35 (m, 1H), 7.32−7.30 (m, 2H), 7.17−7.15 (m, 2H), 6.69−6.67 (m, 2H), 6.28 (d, 1H), 6.17 (d, 1H), 3.73 (s, 2H).

### Example 10

### 6,7-Difluoro-2-(4-hydroxy-phenyl)-3-phenyl-1H-inden-5-ol (E10)

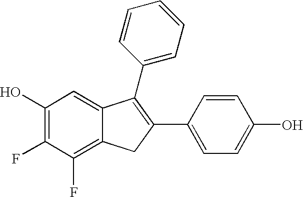

The title compound was synthesized using the procedures described in example 8. EI/MS m/z: 337.4 (pos, M+H), 335.5 (neg, M−H); 1H-NMR (500 MHz, acetone-d₆) δ 7.51 (d, 1H), 7.28−7.24 (m, 1H), 7.23−7.18 (m, 3H), 7.17−7.14 (m, 1H), 6.80−6.77 (m, 1H), 6.77−6.74 (m, 2H), 6.74−6.71 (m, 1H), 3.89 (s, 2H).

### Example 11

### 2-(2-Fluoro-4-hydroxy-phenyl)-7-methyl-3-phenyl-1H-inden-5-ol (E11)

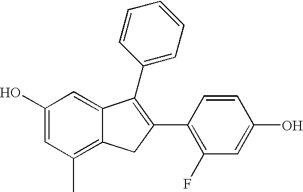

The title compound was synthesized using the procedures described in example 8. ES/MS m/z: 333.7 (pos, M+H), 331.7 (neg, M−H); ¹H NMR (500 MHz, acetone-d₆) δ 7.40−7.36 (m, 2H), 7.33−7.27 (m, 3H), 7.01 (t, 1H), 6.63 (d, 1H), 6.58 (d, 1H), 6.57−6.51 (m, 2H), 3.68 (s, 2H), 2.33 (s, 3H).

### Example 12

### 2-(3-Fluoro-4-hydroxy-phenyl)-7-methyl-3-phenyl-1H-inden-5-ol (E12)

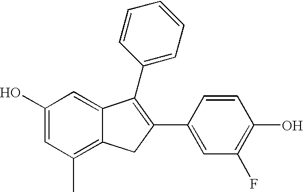

The title compound was synthesized using the procedures described in example 6. ES/MS m/z: 333.7 (pos, M+H), 331.7 (neg, M−H); ¹H NMR (500 MHz, acetone-d₆) δ 7.40−7.36 (m, 2H), 7.33−7.27 (m, 3H), 7.01 (t, 1H), 6.63 (d, 1H), 6.58 (d, 1H), 6.57-6.51 (m, 2H), 3.68 (s, 2H), 2.33 (s, 3H).

### Example 13

### 2-(3-Chloro-4-hydroxy-phenyl)-7-methyl-3-phenyl-1H-inden-5-ol (E13)

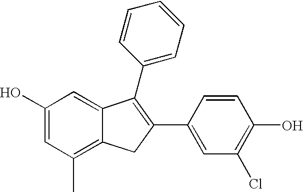

The title compound was synthesized using the procedures described in example 8. ES/MS m/z: 349.3 (pos., M+H), 347.3 (neg., M−H); ¹H NMR (500 MHz, acetone-d₆) δ 7.48 (t, 2H), 7.43−7.38 (m, 1H), 7.34−7.30 (m, 3H), 7.10 (dd, 1H), 6.88 (d, 1H), 6.56 (d, 1H), 6.44 (d, 1H), 3.74 (s, 2H), 2.35 (s, 3H).

### Example 14

### 2-(3-Bromo-4-hydroxy-phenyl)-7-methyl-3-phenyl-1H-inden-5-ol (E14)

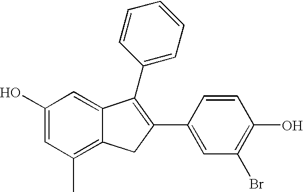

The title compound was synthesized using the procedures described in example 8. ES/MS m/z: 393.2 (pos., M+H), 391.2 (neg., M−H); ¹H NMR (500 MHz, acetone-d₆) δ 7.50−7.46 (m, 3H), 7.43−7.38 (m, 1H), 7.34−7.31 (m, 2H), 7.13 (dd, 1H), 6.91-6.89 (m, 1H), 6.56 (d, 1H), 6.44 (d, 1H), 3.74 (s, 2H), 2.35 (s, 3H).

### Example 15

### 2-(3,5-Dibromo-4-hydroxy-phenyl)-7-methyl-3-phenyl-3H-inden-5-ol (E15)

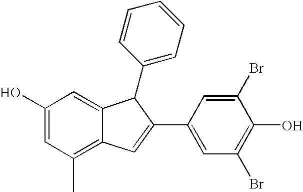

The title compound was synthesized using the procedures described in example 8. ES/MS m/z: 473.2 (pos., M+H), 471.1 (neg., M−H); ¹H NMR (500 MHz, acetone-d₆) δ 7.71 (s, 2H), 7.57 (s, 1H), 7.28−7.23 (m, 2H), 7.21−7.15 (m, 3H), 6.57 (s, 1H), 6.50 (s, 1H), 5.02 (s, 1H), 2.44 (s, 3H).

### Example 16

### 2-(2,5-Dichloro-4-hydroxy-phenyl)-7-methyl-3-phenyl-3/f-inden-5-ol (E16)

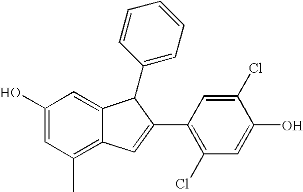

The title compound was synthesized using the procedures described in example 8. ES/MS m/z: 383.3 (pos., M+H), 381.1 (neg., M−H); ¹H NMR (500 MHz, CDCl₃) δ 7.22−7.11 (m, 4H), 7.07−7.00 (m, 3H), 6.58 (d, 1H), 6.52 (d, 1H), 5.48 (s, 1H), 5.15 (s, 1H), 2.45 (s, 3H).

### Example 17

### 2-(4-Hydroxy-phenyl)-7-methyl-3-thiophen-3-yl-1H-inden-5-ol (E17)

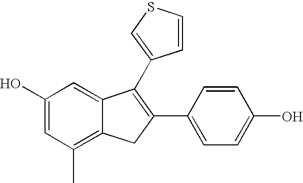

The title compound was synthesized according to the method outlined in Scheme 5.

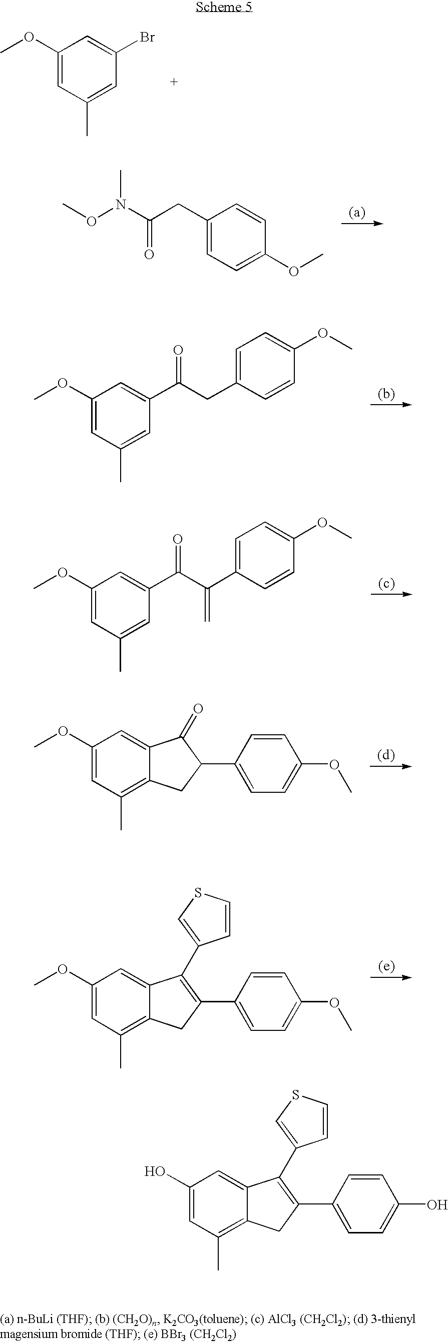

Step (a): 1 mmol of 3-bromo-5-methyl-anisole was dissolved in 8 ml THF and cooled to −78° C. 1 mmol of n-BuLi was slowly added and the mixture was stirred for 15 min. 0.85 mmol of N-methoxy-2-(4-methoxy-phenyl)-N-methyl-acetamide in 2 ml TH were added and the mixture was allowed to slowly reach room temperature. 1M HCl and dichloromethane were added and the phases were separated. After flash chromatographic separation 0.33 mmol of 1-(3-methoxy-5-methyl-phenyl)-2-(4-methoxy-phenyl)-ethanone were obtained.

Step (b): To 0.33 mmol 1-(3-methoxy-5-methyl-phenyl)-2-(4-methoxy-phenyl)-ethanone in 10 ml toluene were added 6.6 mmol paraformaldehyde and 0.66 mmol potassium carbonate. The mixture was heated to 85° C. over night. 1M HCl and dichloromethane were added and the phases were separated. 0.33 mmol of 1-(3-methoxy-5-methyl-phenyl)-2-(4-methoxy-phenyl)-propenone were obtained and used without further purification.

Step (c): 0.16 mmol 1-(3-Methoxy-5-methyl-phenyl)-2-(4-methoxy-phenyl)-propenone were dissolved in 2 ml dichloromethane and 0.48 mmol AlCl₃ were added. The mixture was stirred for 15 min at room temperature. 1M HCl and dichloromethane were added and the phases were separated. After flash chromatographic separation 0.11 mmol of 6-methoxy-2-(4-methoxy-phenyl)-4-methy-indan-1-one were obtained.

Step (d): 0.213 mmol BF3*Et₂O were added to 0.053 mmol of 6-methoxy-2-(4-methoxy-phenyl)-4-methyl-indan-1-one in 2 ml THF at 0° C. After 30 min 0.213 mmol 3-thienyl magnesium bromide (0.3 M in THF) were added and the mixture was stirred over night at room temperature. 1M HCl and dichloromethane were added and the phases were separated. After flash chromatographic separation 0.034 mmol of 6-methoxy-2-(4-methoxy-phenyl)-4-methyl-indan-1-one were obtained.

Step (e): 0.034 mmol 6-methoxy-2-(4-methoxy-phenyl)-4-methyl-indan-1-one were dissolved in 2 ml dichloromethane and cooled to 0° C. 0.34 mmol BBr₃ (1M in dichloromethane) were added and the mixture was allowed to warm to room temperature over night. 1M HCl and dichloromethane were added and the phases were separated. After flash chromatographic separation 0.006 mmol of 2-(4-hydroxy-phenyl)-7-methyl-3-thiophen-3-yl-1H-inden-5-ol were obtained. ES/MS m/z: 321.2 (pos, M+H); ¹H NMR (500 MHz, CDCl₃) δ 7.49 (dd, 1H), 7.33 (dd, 1H), 7.14-7.18 (m, 2H), 6.93 (dd, 1H), 6.64-6.69 (m, 2H), 6.50 (d, 1H), 6.46 (d, 1H), 3.62 (s, 2H), 2.26 (s, 3H).

### Example 18

### 2-(4-Hydroxy-phenyl)-7-methyl-3-thiophen-2-yl-1H-inden-5-ol (E18)

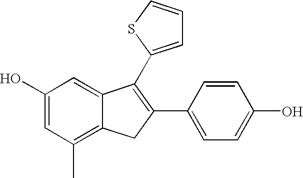

The title compound was synthesized using the procedures described in example 17. ES/MS m/z: 321.2 (pos, M+H); ¹H NMR (500 MHz, CDCl₃) δ 7.40 (dd, 1H), 7.12-7.16 (m, 2H), 7.03 (dd, 1H), 6.93 (dd, 1H), 6.60-6.64 (m, 2H), 6.50 (d, 1H), 6.42 (d, 1H), 3.58 (s, 2H), 2.20 (s, 3H).

### Example 19

### 7-Bromo-2-(4-hydroxy-phenyl)-3-phenyl-1H-inden-5-ol (E19)

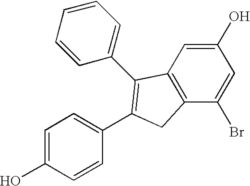

The title compound was synthesized using the procedures described in example 17. ES/MS m/z: 379.4 (pos, M+H); ¹H NMR (500 MHz, d⁶-acetone) δ 7.40-7.53 (m, 3H), 7.30-7.36 (m, 2H), 7.17-7.24 (m, 2H), 6.88 (d, 1H), 6.69-6.75 (m, 2H), 6.57 (d, 1H), 3.80 (s, 2H).

### Example 20

### 6-Hydroxy-2-(4-hydroxy-phenyl)-1-phenyl-3H-indene-4-carbonitrile (E20)

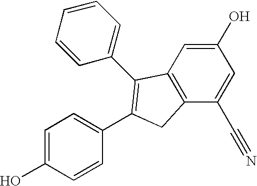

The title compound was synthesized using the procedures described in example 17. ES/MS m/z: 326.3 (pos, M+H), 324.4 (neg, M−H); ¹H-NMR (500 MHz, CDCl₃) δ 7.47−7.42 (m, 2H), 7.41−7.38 (m, 1H), 7.32−7.29 (m, 2H), 7.19−7.17 (m, 2H), 6.91 (d, 1H), 6.83 (d, 1H), 6.69 (d, 2H), 4.86 (s, OH), 4.75 (s, OH), 3.99 (s, 2H).

### Example 21

### 6-Hydroxy-2-(4-hydroxy-phenyl)-1-phenyl-3H-indenecarbaldehyde (E21)

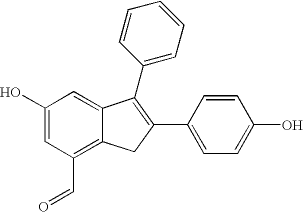

The title compound was synthesized using the procedures described in example 17. ES/MS m/z: 329.3 (pos, M+H), 327.4 (neg, M−H); ¹H-NMR (500 MHz, CDCl₃) δ 10.24 (s, 1H), 7.52−7.47 (m, 2H), 7.44−7.40 (m, 1H), 7.37−7.34 (m, 2H), 7.25−7.22 (m, 2H), 7.20 (d, 1H), 6.86 (d, 1H), 6.74−6.71 (m, 2H), 4.17 (s, 2H).

### Example 22

### 2-(4-Hydroxy-phenyl)-1-butyl-3-phenyl-3H-inden-5-ol (E22)

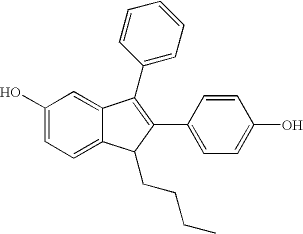

The title compound was synthesized according to the methods outlined in Scheme 6.

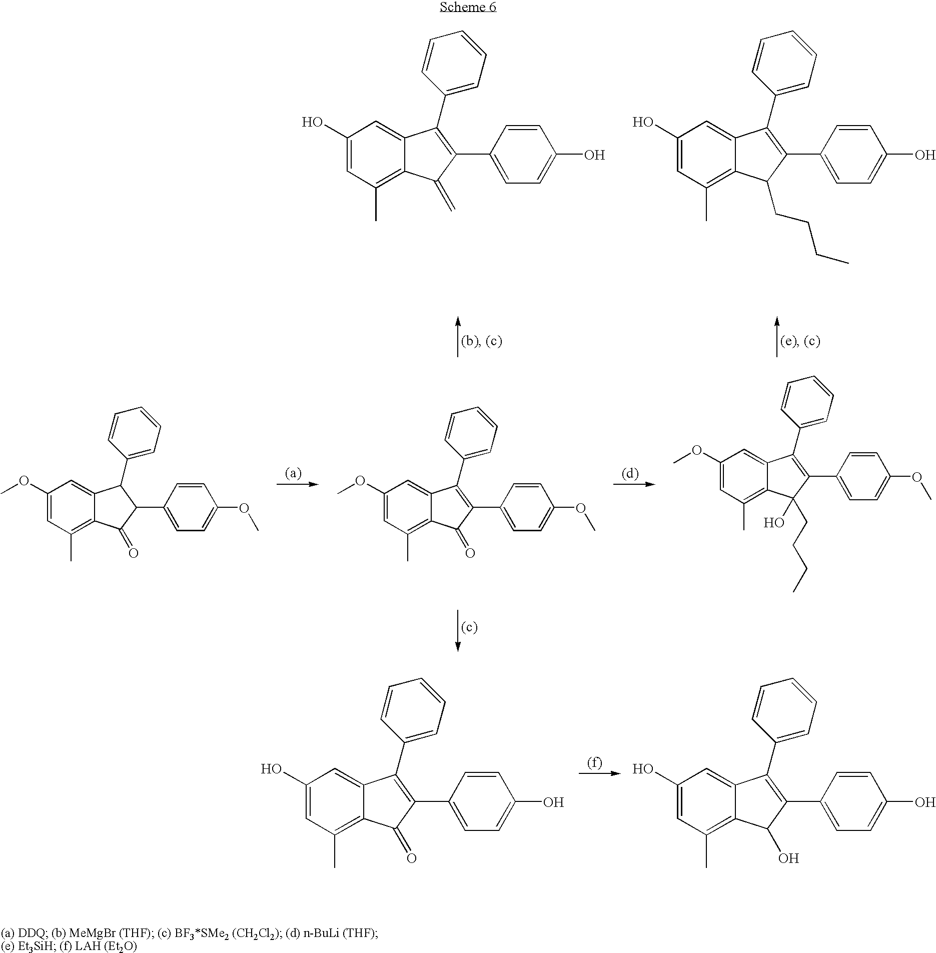

Step (a): 0.05 mmol 5-methoxy-2-(4-methoxy-phenyl)-7-methyl-3-phenyl-indan-1-one were dissolved in 1 ml dioxane. 0.1 mmol DDQ were added and the mixture was warmed to 100° C. for 4 h. 1M HCl and dichloromethane were added and the phases were separated. After flash chromatographic separation 0.023 mmol of 5-methoxy-2-(4-methoxy-phenyl)-7-methyl-3-phenyl-inden-1-one were obtained.

Step (b): 0.084 mmol of methyl magnesium bromide (3M in THF) were added to 0.042 mmol 5-methoxy-2-(4-methoxy-phenyl)-7-methyl-3-phenyl-inden-1-one in 2 ml THF at 0° C. After 60 min 1M HCl and dichloromethane were added and the phases were separated. The crude material was dissolved in 2 ml ethanol and 2 drops of HCl (conc.) were added. The mixture was stirred for 60 min at room temperature. Water and dichloromethane were added and the phases were separated. 0.04 mmol of 5-methoxy-2-(4-methoxy-phenyl)-7-methyl-1-methylene-3-phenyl-1H-indene were obtained and were used without further purification.

Step (c): 0.039 mmol 5-methoxy-2-(4-methoxy-phenyl)-7-methyl-1-methylene-3-phenyl-1H-indene were dissolved in 2 ml dichloromethane and cooled to 0° C. 0.39 mmol BF₃*SMe₂ (1M in dichloromethane) were added and the mixture was allowed to warm to room temperature over night. 1M HCl and dichloromethane were added and the phases were separated. After flash chromatographic separation 0.018 mmol of 2-(4-hydroxy-phenyl)-7-methyl-1-methylene-3-phenyl-1-inden-5-ol were obtained.

Step (d), (e): 0.1 mmol of 5-methoxy-2-(4-methoxy-phenyl)-7-methyl-3-phenyl-inden-1-one were dissolved in 2 ml THF and cooled to 0° C. 0.2 mmol n-BuLi were added and the mixture was allowed to slowly warm to room temperature over night. 1M HCl and dichloromethane were added and the phases were separated. The crude product was dissolved in 2 ml dichloromethane. 0.2 mmol triethyl silane and 0.4 mmol trifluoroacetic anhydride were added and the reaction was stirred over night. 1M HCl and dichloromethane were added and the phases were separated. After flash chromatographic separation 0.05 mmol of 1-butyl-5-methoxy-2-(4-methoxy-phenyl)-7-methyl-3-phenyl-1H-indene were obtained.

Step (f): 0.014 mmol of 5-hydroxy-2-(4-hydroxy-phenyl)-7-methyl-3-phenyl-inden-1-one were dissolved in 2 ml diethylether and cooled to 0° C. 0.014 mmol lithium aluminium hydride were added and the reaction was stirred for 30 min. 1M HCl and dichloromethane were added and the phases were separated. After flash chromatographic separation 0.01 mmol of 2-(4-hydroxy-phenyl)-7-methyl-3-phenyl-1H-indene-1,5-diol were obtained. ES/MS m/z: 357.2 (pos, M+H), 355.3 (neg, M−H); ¹H NMR (500 MHz, d⁶-acetone) δ 7.39-7.45 (m, 2H), 7.29-7.38 (m, 4H), 7.06-7.10 (m, 2H), 6.73-6.77 (m, 2H), 6.72 (d, 1H), 6.67 (d, 1H), 4.06 (t, 1H), 1.94-2.00 (m, 1H), 1.60-1.68 (m, 1H), 1.00-1.18 (m, 4H), 0.72 (t, 3H).

### Example 23

### 2-(4-Hydroxy-phenyl)-7-methyl-3-phenyl-1H-indene-1,5-diol (E23)

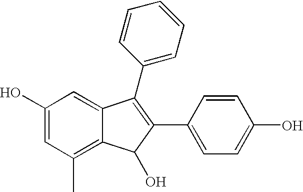

The title compound was synthesized according to the methods outlined in Scheme 6. ES/MS m/z: 329.3 (neg, M−H); ¹H NMR (500 MHz, d⁶-acetone) δ 7.28-7.33 (m, 2H), 7.21-7.26 (m, 1H), 7.15-7.18 (m, 2H), 7.07-7.11 (m, 2H), 6.53-6.58 (m, 2H), 6.34 (d, 1H), 6.20 (d, 1H), 5.45 (d, 1H), 2.31 (s, 3H).

### Example 24

### 2-(4-Hydroxy-phenyl)-7-methyl-1-methylene-3-phenyl-1H-inden-5-ol (E24)

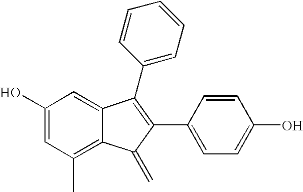

The title compound was synthesized according to the methods outlined in Scheme 6. ES/MS m/z: 327.2 (pos, M+H), 325.3 (neg, M−H); ¹H NMR (500 MHz, d⁶-acetone) δ 7.10-7.23 (5, 5H), 6.86-6.90 (m, 2H), 6.63-6.68 (m, 2H), 6.50 (d, 1H), 6.43 (d, 1H), 5.98 (s, 1H), 5.49 (s, 1H), 2.40 (s, 3H).

### Example 25

### 2-(4-Hydroxy-phenyl)-1-methyl-3-phenyl-3H-inden-5-ol (E25)

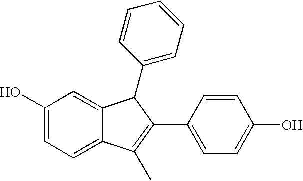

The title compound was synthesized according to the method outlined in Scheme 7.

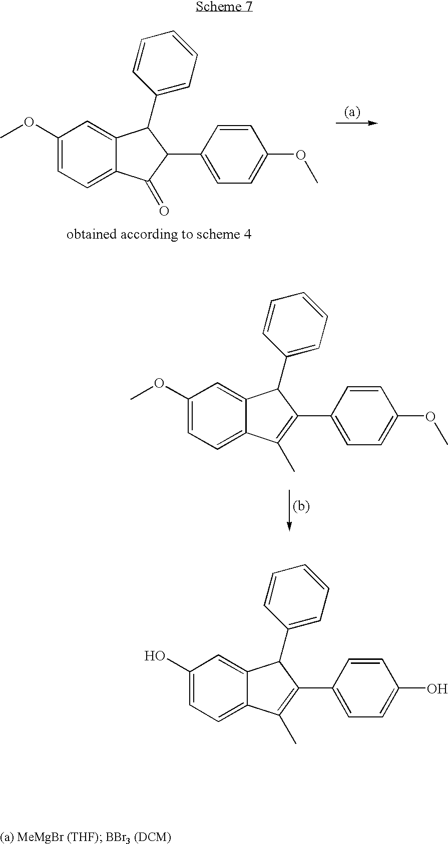

Step (a): 0.055 mmol of 5-methoxy-2-(4-methoxy-phenyl)-3-phenyl-indan-1-one were dissolved in 2 ml diethylether and 0.165 mmol of methyl magnesium bromide were added at 0° C. The reaction was stirred at room temperature over night. 1M HCl and dichloromethane were added and the phases were separated. After flash chromatographic separation 0.05 mmol of 6-methoxy-2-(4-methoxy-phenyl)-3-methyl-1-phenyl-1H-indene were obtained.

Step (b): 0.05 mmol 6-methoxy-2-(4-methoxy-phenyl)-3-methyl-1-phenyl-1H-indene were dissolved in 3 ml dichloromethane and cooled to 0° C. 0.5 mmol BBr₃ (1M in dichloromethane) were added and the mixture was allowed to warm to room temperature over night. 1M HCl and dichloromethane were added and the phases were separated. After flash chromatographic separation 0.025 mmol of 2-(4-hydroxy-phenyl)-1-methyl-3-phenyl-3H-inden-5-ol were obtained. ES/MS m/z: 315.4 (pos, M+H), 313.3 (neg, M−H); ¹H NMR (500 MHz, d⁶-acetone) δ 8.47 (s, 1H, OH), 8.25 (s, 1H, OH), 7.47 (d, 2H), 7.44 (d, 1H), 7.36-7.40 (m, 2H), 7.28-7.23 (m, 3H), 6.89-7.03 (m, 3H), 6.89 (d, 1H), 5.21 (s, 1H), 2.51 (d, 3H).

### Example 26

### 2-(4-Hydroxy-phenyl)-1-isobutyl-3-phenyl-3H-inden-5-ol (E26)

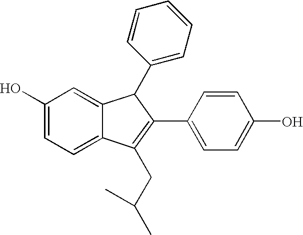

The title compound was synthesized using the procedures described in example 25. ES/MS m/z: 357.2 (pos, M+H), 355.3 (neg, M−H); ¹H NMR (500 MHz, d⁶-acetone) δ 8.10 (s, 1H, OH), 7.89 (s, 1H, OH), 7.12 (d, 1H), 7.05 (d, 2H), 6.99-7.04 (m, 2H), 6.90-6.96 (m, 3H), 6.59-6.66 (m, 3H), 6.51 (d, 1H), 4.80 (s, 1H), 2.5 (ddd, 2H), 1.95-2.04 (m, 2H), 0.85 (d, 3H), 0.73 (d, 3H).

### Example 27

### 2-(4-Hydroxy-phenyl)-1-butyl-3-phenyl-3H-inden-5-ol (E27)

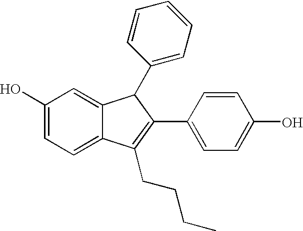

The title compound was synthesized using the procedures described in example 25. ES/MS m/z: 357.2 (pos, M+H), 355.3 (neg, M−H); ¹H NMR (500 MHz, d⁶-acetone) δ 8.10 (s, 1H, OH), 7.88 (s, 1H, OH), 7.11 (d, 1H), 7.07 (d, 2H), 6.99-7.04 (m, 2H), 6.90-6.96 (m, 3H), 6.60-6.70 (m, 3H), 6.52 (d, 1H), 4.82 (s, 1H), 2.57 (t, 2H), 1.50-1.70 (m, 2H), 1.30-1.39 (m, 2H), 0.82 (1, 3H).

### Example 28

### 2-(4-Hydroxy-phenyl)-1-ethyl-3-phenyl-3H-inden-5-ol (E28)

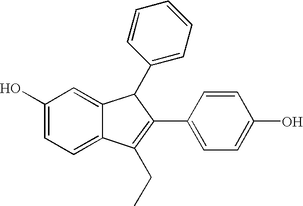

The title compound was synthesized using the procedures described in example 25. ES/MS m/z: 329.3 (pos, M+H), 327.4 (neg, M−H); ¹H NMR (500 MHz, d⁶-acetone) a 8.05 (s, 1H, OH), 7.84 (s, 1H, OH), 7.07 (d, 1H), 7.02 (d, 2H), 6.94-6.99 (m, 2H), 6.85-6.91 (m, 3H), 6.55-6.63 (m, 3H), 6.47 (d, 1H), 4.77 (s, 1H), 2.49-2.60 (m, 2H), 1.16 (t, 3H).

### Example 29

### 2-(4-Hydroxy-phenyl)-1-propyl-3-phenyl-3H-inden-5-ol (E29)

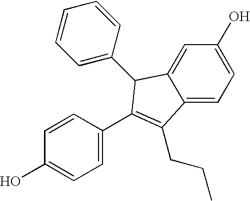

The title compound was synthesized using the procedures described in example 25. ES/MS m/z: 343.1 (pos, M+H), 341.2 (neg, M−H); ¹H NMR (500 MHz, d⁶-acetone) δ 8.11 (s, 1H, OH), 7.88 (s, 1H, OH), 7.11 (d, 1H), 7.05 (d, 2H), 6.98-7.03 (m, 2H), 6.90-6.96 (m, 3H), 6.60-6.67 (m, 3H), 6.51 (d, 1H), 4.82 (s, 1H), 2.55 (t, 2H), 1.55-1.73 (m, 2H), 0.90 (t, 3H).

### Example 30

### 2-(4-Hydroxy-phenyl)-1-pentyl-3-phenyl-3H-inden-5-ol (E30)

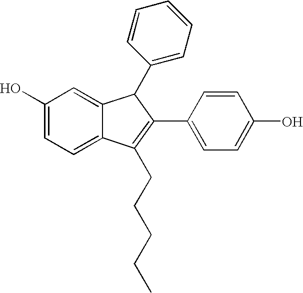

The title compound was synthesized using the procedures described in example 25. ES/MS m/z: 371.3 (pos, M+H), 369.1 (neg, M−H); ¹H NMR (500 MHz, d⁶-acetone) δ 8.10 (s, 1H, OH), 7.88 (s, 1H, OH), 7.11 (d, 1H), 7.06 (d, 2H), 6.99-7.03 (m, 2H), 6.90-6.96 (m, 3H), 6.60-6.67 (m, 3H), 6.52 (d, 1H), 4.82 (s, 1H), 2.57 (t, 2H), 1.52-1.75 (m, 2H), 1.20-1.35 (m, 4H), 0.77 (t, 3H).

### Example 31

### 2-(4-hydroxy-phenyl)-3-(2-cyano-phenyl)-1H-inden-5-ol (E31)

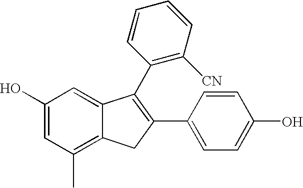

The title compound was synthesized according to the method outlined in Scheme 8.

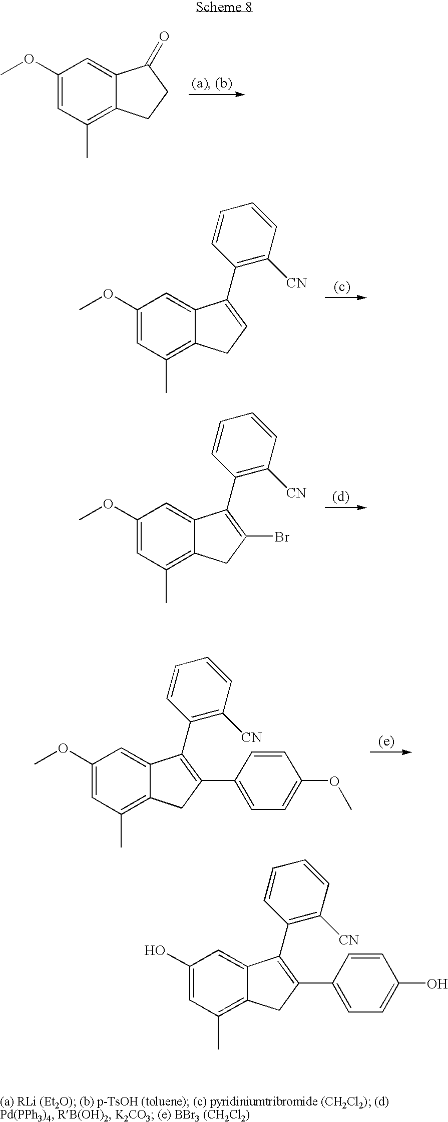

Step (a), (b): 0.17 mmol of 6-methoxy-4-methyl-indanone in 2 ml diethylether was added to 0.34 mmol of 2-cyano-phenyllithium in 4 ml diethylether at −78° C. The reaction was allowed to warm to room temperature overnight and 1M HCl and dichloromethane were added and the phases were separated. The crude mixture was dissolved in 2 ml toluene. 5 mg p-TsOH were added and the mixture was heated to 80° C. for 30 min. After flash chromatographic separation, 0.075 mmol of 5-methoxy-7-methyl-3-(2-cyano-phenyl)-1H-indene was obtained.

Step (c): 0.075 mmol of 5-methoxy-7-methyl-3-(2-cyano-phenyl)-1H-indene was dissolved in 2 ml dichloromethane and cooled to 0° C. 0.075 mmol of pyridinium tribromide were added and the mixture was stirred for 15 min. 1M HCl and dichloromethane were added and the phases were separated. The crude product (0.061 mmol) was used without further purification in the next step.

Step (d): The crude product from step (c) was dissolved in 2.2 ml EtOH (95%)/toluene (10:1) and 0.006 mmol of tetrakis-triphenylphosphine-palladium(0), 0.12 potassium carbonate and 0.12 p-methoxy-phenyl-boronic acid were added. The mixture was heated to 140° C. in the microwave for 10 min. 1M HCl and dichloromethane were added and the phases were separated. After flash chromatographic separation 0.03 mmol of 5-methoxy-7-methyl-2-(4-methoxy-phenyl)-3-(2-cyano-phenyl)-1H-indene were obtained.

Step (e): To 0.03 mmol of 5-methoxy-7-methyl-2-(4-methoxy-phenyl)-3-(2-cyano-phenyl)-1H-indene in 2 ml dichloromethane were added 0.3 mmol BBr₃ (1M solution in dichloromethane) at 0° C. The mixture was allowed to slowly warm to room temperature. After a total reaction time of 3 h 1M HCl and dichloromethane were added and the phases were separated. After flash chromatographic separation 0.015 mmol of 2-(4-hydroxy-phenyl)-3-(2-cyano-phenyl)-1H-inden-5-ol were obtained. ES/MS m/z: 341.3 (pos, M+H), 339.1 (neg, M−H); ¹H NMR (500 MHz, CDCl₃) δ 8.06 (d, 1H), 7.80 (dt, 1H), 7.54 (d, 1H), 7.47 (t, 1H), 6.92 (d, 2H), 6.67 (d, 1H), 6.63 (dd, 1H), 6.48 (d, 1H), 3.54 (d, 1H), 3.39 (d, 1H), 2.12 (s, 3H).

### Example 32

### 4-[6-Hydroxy-2-(4-hydroxy-phenyl)-4-methyl-3H-inden-1-yl]-thiophene-3-carbonitrile (E32)

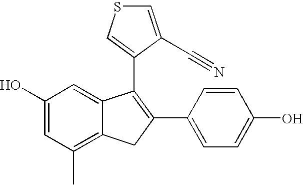

The title compound was synthesized using the procedures described in example 31.

ES/MS m/z: 346.1 (pos, M+H), 343.9 (neg, M−H); ¹H NMR (500 MHz, acetone) δ 8.27 (d, 1H), 7.45 (d, 1H), 6.98 (d, 2H), 6.54 (d, 2H), 6.35 (d, 1H), 6.18 (d, 1H), 3.66 (s, br, 1H), 3.50 (s, br, 1H), 2.14 (s, 3H).

### Example 33

### 2-(4-Hydroxy-phenyl)-7-methyl-3-(3-methy)thiophen-2-yl)-1H-inden-5-ol (E33)

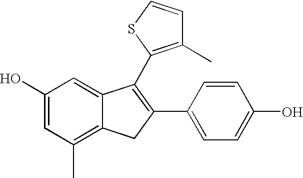

The title compound was synthesized using the procedures described in example 31.

ES/MS m/z: 335.4 (pos, M+H); ¹H NMR (500 MHz, acetone-dd) δ 7.71 (d, 1H), 7.48-7.53 (m, 2H), 7.25 (d, 1H), 6.95-7.00 (m, 2H), 6.77 (dd, 1H), 6.69 (d, 1H), 3.86-4.15 (br m, 2H), 2.57 (s, 3H).

### Example 34

### 2-(2,3-Difluoro-4-hydroxy-phenyl)-7-methyl-3-phenyl-1H-inden-5-ol (E34)

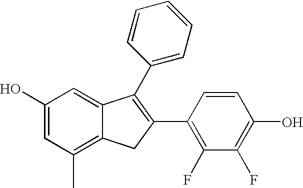

The title compound was synthesized using the procedures described in example 31.

ES/MS m/z: 350.4 (pos, M+H); ¹H NMR (500 MHz, acetone-d₆) δ 7.15-7.20 (m, 2H), 7.05-7.13 (m, 3H), 6.59-6.65 (m, 1H), 6.44-6.50 (m, 1H), 6.42 (d, 1H), 6.38 (d, 1H), 3.49 (s, 2H), 2.12 (s, 3H).

### Example 35

### 2-(2-Chloro-4-hydroxy-phenyl)-7-methyl-3-phenyl-1H-inden-5-ol (E35)

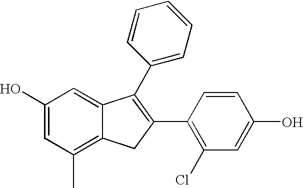

The title compound was synthesized using the procedures described in example 31.

ES/MS m/z: 349.2 (pos, M+H); ¹H NMR (500 MHz, acetone-d₆) δ 7.17-7.22 (m, 2H), 7.10-7.15 (m, 3H), 6.97 (d, 1H), 6.76 (d, 1H), 6.55-6.60 (m, 2H), 6.47 (d, 1H), 3.50 (s, 2H), 2.19 (s, 3H).

### Example 36

### 2-(4-Hydroxy-3-methyl-phenyl)-7-methyl-3-phenyl-1H-inden-5-ol (E36)

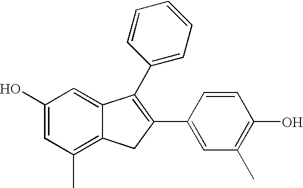

The title compound was synthesized using the procedures described in example 31.

ES/MS m/z: 329.5 (pos, M+H); ¹H NMR (500 MHz, acetone-d₆) δ 7.53-7.59 (m, 2H), 7.46-7.51 (m, 1H), 7.40-7.44 (m, 2H), 7.23 (d, 1H), 7.06 (dd, 1H), 6.75 (dd, 1H), 6.63 (d, 1H), 6.53 (d, 1H), 3.81 (s, 2H), 2.44 (s, 3H).

### Example 37

### 2-(4-Hydroxy-2-methyl-phenyl)-7-methyl-3-phenyl-1H-inden-5-ol (E37)

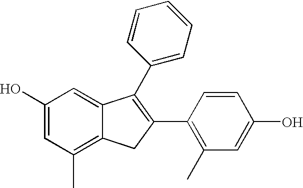

The title compound was synthesized using the procedures described in example 31.

ES/MS m/z: 329.5 (pos, M+H); ¹H NMR (500 MHz, acetone-d₆) δ 7.18-7.23 (m, 2H), 7.11-7.16 (m, 3H), 7.00 (dd, 1H), 6.62 (d, 1H), 6.55 (d, 1H), 6.49 (d, 1H), 6.47 (d, 1H), 3.45 (s, 2H), 2.21 (s, 3H).

### Example 38

### 3-[6-Hydroxy-2-(4-hydroxy-phenyl)-4-methyl-3H-inden-1-yl]-thiophene-2-carbonitrile (E38)

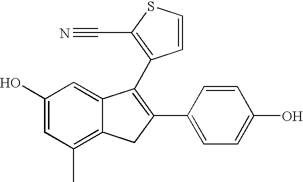

The title compound was synthesized using the procedures described in example 31.

ES/MS m/z: 346.1 (pos, M+H); ¹H NMR (500 MHz, acetone-d₆) δ 7.90 (d, 1H), 7.04 (d, 1H), 6.95-7.00 (m, 2H), 6.55-6.59 (m, 2H), 6.38 (d, 1H), 6.21 (d, 1H), 3.52-3.70 (br s, 2H), 2.15 (s, 3H).

### Example 39

### 2-(4-Hydroxy-phenyl)-7-methyl-3-thiazol-5-yl-1H-inden-5-ol (E39)

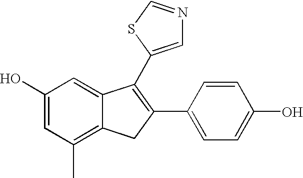

The title compound was synthesized using the procedures described in example 31.

ES/MS m/z: 322.4 (pos, M+H); ¹H NMR (500 MHz, acetone-d₆) δ 10.22 (s, 1H), 8.54 (d, 1H), 7.21-7.25 (m, 2H), 6.73-6.78 (m, 2H), 6.61 (d, 1H), 6.58 (d, 1H), 3.71 (s, 2H), 2.22 (s, 3H).

### Example 40

### 3-(2-Ethyl-phenyl)-2-(4-hydroxy-phenyl)-7-methyl-1H-inden-5-ol (E40)

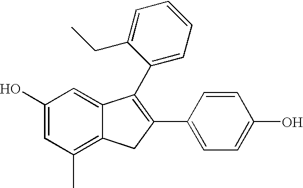

The title compound was synthesized using the procedures described in example 31.

ES/MS m/z: 343.4 (pos, M+H); ¹H NMR (500 MHz, acetone-d₆) δ 7.15-7.23 (m, 2H), 7.10 (dt, 1H), 6.97-7.03 (m, 2H), 6.94 (d, 1H), 6.43-6.49 (m, 2H), 6.31 (d, 1H), 5.91 (d, 1H), 3.58 (dd, 2H), 2.16-2.31 (m, 2H), 2.15 (s, 3H), 0.76 (t, 3H).

### Example 41

### 2-(4-Hydroxy-phenyl)-3-(2-isopropyl-phenyl)-7-methyl-1H-inden-5-ol (E41)

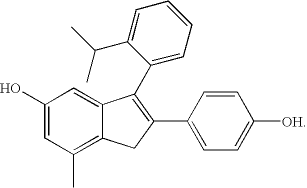

The title compound was synthesized using the procedures described in example 31.

ES/MS m/z: 357.2 (pos, M+H); ¹H NMR (500 MHz, acetone-d₆) δ 7.35 (d, 1H), 7.29 (dt, 1H), 7.16 (dt, 1H), 7.05-7.09 (m, 2H), 6.99 (dd, 1H), 6.51-6.56 (m, 2H), 6.38 (d, 1H), 5.99 (d, 1H), 3.65 (dd, 2H), 2.77 (hept, 1H), 2.22 (s, 3H), 0.93 (d, 3H), 0.81 (d, 3H).

### Example 42

### 3-[2-(3-Fluoro-4-hydroxy-phenyl)-6-hydroxy-4-methyl-3H-inden-1-yl]-thiophene-2-carbonitrile (E42)

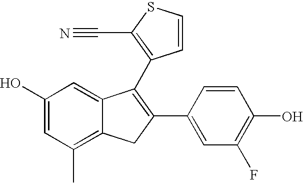

The title compound was synthesized using the procedures described in example 31.

ES/MS m/z: 364.3 (pos, M+H); ¹H NMR (500 MHz, acetone-d₆) δ 7.98 (d, 1H), 7.12 (d, 1H), 6.86 (dd, 1H), 6.81 (dd, 1H), 6.76 (t, 1H), 6.44 (d, 1H), 6.25 (d, 1H), 3.66 (br s, 2H), 2.19 (s, 3H).

### Example 43

### 3-[2-(3-Chloro-4-hydroxy-phenyl)-6-hydroxy-4-methyl-3H-inden-1-yl]-thiophene-2-carbonitrile (E43)

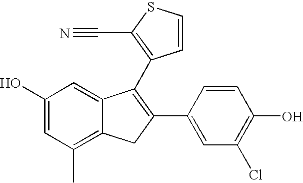

The title compound was synthesized using the procedures described in example 31.

ES/MS m/z: 380.2 (pos, M+H); ¹H NMR (500 MHz, acetone-d₆) δ 8.00 (dd, 1H), 7.14-7.17 (m, 2H), 6.95 (dt, 1H), 6.80 (dd, 1H), 6.47 (s, 1H), 6.30 (s, 1H), 3.70 (br s, 2H), 2.22 (s, 3H).

### Example 44

### 3-[2-(2,3-Difluoro-4-hydroxy-phenyl)-6-hydroxy-4-methyl-3H-inden-1-yl]-thiophene-2-carbonitrile (E44)

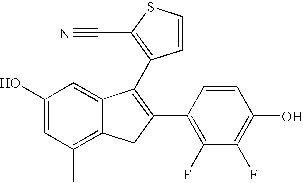

The title compound was synthesized using the procedures described in example 31.

ES/MS m/z: 382.2 (pos, M+H); ¹H NMR (500 MHz, acetone-d₆) δ 7.86 (d, 1H), 7.13 (d, 1H), 6.65 (dt, 1H), 6.58 (dt, 1H), 6.45 (d, 1H), 6.39 (d, 1H), 3.62 (br s, 2H), 2.16 (s, 3H).

### Example 45

### 3-[6-Hydroxy-2-(4-hydroxy-phenyl)-4-trifluoromethyl-3H-inden-1-yl]-thiophene-2-carbonitrile (E45)

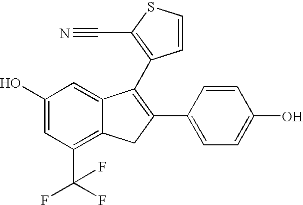

The title compound was synthesized using the procedures described in example 31.

ES/MS m/z: 400.2 (pos, M+H); ¹H NMR (500 MHz, acetone-d₆) δ 8.01 (d, 1H), 7.15 (d, 1H), 7.05-7.10 (m, 2H), 6.85 (d, 1H), 6.63-6.68 (m, 3H), 3.96 (br s, 2H).

### Example 46

### 3-[2-(3-Fluoro-4-hydroxy-phenyl)-6-hydroxy-4-trifluoromethyl-3H-inden-1-yl]-thiophene-2-carbonitrile (E46)

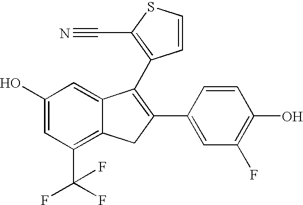

The title compound was synthesized using the procedures described in example 31.

ES/MS m/z: 418.4 (pos, M+H); ¹H NMR (500 MHz, acetone-d₆) δ 8.05 (d, 1H), 7.20 (d, 1H), 6.96 (dd, 1H), 6.86-6.92 (m, 2H), 6.81 (t, 1H), 6.67 (s, 1H), 4.00 (br s, 2H).

### Example 47

### 3-[2-(3-Chloro-4-hydroxy-phenyl)-6-hydroxy-4-trifluoromethyl-3H-inden-1-yl]-thiophene-2-carbonitrile (E47)

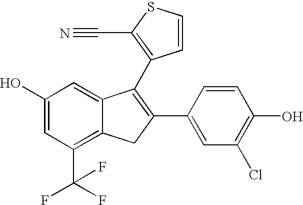

The title compound was synthesized using the procedures described in example 31.

ES/MS m/z: 435.5 (pos, M+H); ¹H NMR (500 MHz, acetone-d₆) δ 8.05 (d, 1H), 7.19-7.23 (m, 2H), 7.01 (dd, 1H), 6.88 (d, 1H), 6.82 (d, 1H), 6.68 (s, 1H), 4.02 (br s, 2H).

### Example 48

### 4-[2-(2,3-Difluoro-4-hydroxy-phenyl)-6-hydroxy-4-methyl-3H-inden-1-yl]-thiophene-3-carbonitrile (E48)

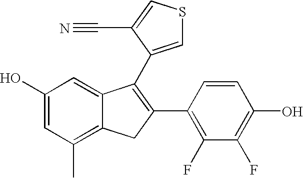

The title compound was synthesized using the procedures described in example 31.

ES/MS m/z: 382.4 (pos, M+H); ¹H NMR (500 MHz, acetone-d₆) δ 8.28 (d, 1H), 7.55 (d, 1H), 6.69 (dt, 1H), 6.60 (dt, 1H), 6.50 (d, 1H), 6.45 (s, 1H), 3.66 (br s, 2H), 2.22 (s, 3H).

### Example 49

### 4-[6-Hydroxy-2-(4-hydroxy-phenyl)-4-trifluoromethyl-3H-inden-1-yl]-thiophene-3-carbonitrile (E49)

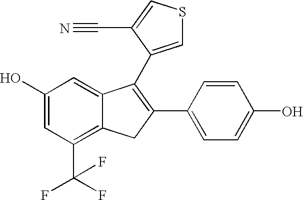

The title compound was synthesized using the procedures described in example 31.

ES/MS m/z: 400.1 (pos, M+H); ¹H NMR (500 MHz, acetone-d₆) 8.55 (d, 1H), 7.77 (d, 1H), 7.21-7.28 (m, 2H), 6.98 (d, 1H), 6.76-6.83 (m, 3H), 3.98-4.20 (br m, 2H).

### Example 50

### 4-[2-(3-Fluoro-4-hydroxy-phenyl)-6-hydroxy-4-trifluoromethyl-3H-inden-1-yl]-thiophene-3-carbonitrile (E50)

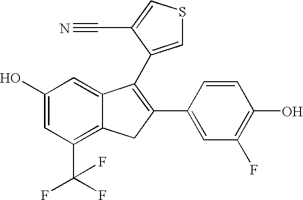

The title compound was synthesized using the procedures described in example 31.

ES/MS m/z: 418.4 (pos, M+H); ¹H NMR (500 MHz, acetone-d₆) δ 8.51 (d, 1H), 7.76 (d, 1H), 7.04 (dd, 1H), 6.99 (ddd, 1H), 6.94 (d, 1H), 6.87 (t, 1H), 6.74 (d, 1H), 3.85-4.25 (br m, 2H).

### Example 51

### 4-[2-(2,3-Difluoro-4-hydroxy-phenyl)-6-hydroxy-4-trifluoromethyl-3H-inden-1-yl]-thiophene-3-carbonitrile (E51)

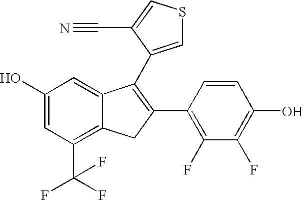

The title compound was synthesized using the procedures described in example 31.

ES/MS m/z: 434.4 (neg, M−H); ¹H NMR (500 MHz, acetone-d₆) δ 8.34 (d, 1H), 7.65 (d, 1H), 6.92 (d, 1H), 6.85 (d, 1H), 6.76 (dt, 1H), 6.63 (dt, 1H), 3.97 (br s, 2H).

### Example 52

### 3-[2-(2,3-Difluoro-4-hydroxy-phenyl)-6-hydroxy-4-trifluoromethyl-3H-inden-1-yl]-thiophene-2-carbonitrile E52)

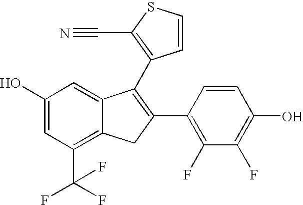

The title compound was synthesized using the procedures described in example 31.

ES/MS m/z: 434.3 (neg, M−H); ¹H NMR (500 MHz, acetone-d₆) δ 7.97 (d, 1H), 7.21 (d, 1H), 6.94 (d, 1H), 6.85 (d, 1H), 6.79 (dt, 1H), 6.68 (dt, 1H), 3.97 (br s, 2H).

### Example 53

### 3-[2-(2,5-Difluoro-4-hydroxy-phenyl)-6-hydroxy-4-trifluoromethyl-3H-inden-1-yl]-thiophene-2-carbonitrile (E53)

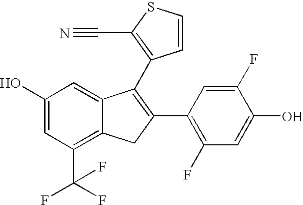

The title compound was synthesized using the procedures described in example 31.

ES/MS m/z: 434.2 (neg, M−H); ¹H NMR (500 MHz, acetone-d₆) δ 7.98 (d, 1H), 7.21 (d, 1H), 6.90-6.96 (m, 2H), 6.84 (d, 1H), 6.65 (dd, 1H), 3.98 (br s, 2H).

### Example 54

### 3-[2-(2,6-Difluoro-4-hydroxy-phenyl)-6-hydroxy-4-trifluoromethyl-3H-inden-1-yl]-thiophene-2-carbonitrile (E54)

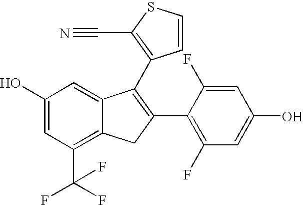

The title compound was synthesized using the procedures described in example 31.

ES/MS m/z: 434.2 (neg, M−H); ¹H NMR (500 MHz, acetone-d₆) δ 7.93 (d, 1H), 7.19 (d, 1H), 6.95 (d, 1H), 6.90 (d, 1H), 6.33-6.39 (m, 2H), 3.91 (br s, 2H).

### Example 55

### 4-[2-(2,5-Difluoro-4-hydroxy-phenyl)-6-hydroxy trifluoromethyl-3H-inden-1-yl]-thiophene-3-carbonitrile (E55)

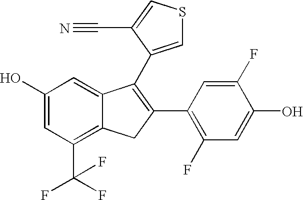

The title compound was synthesized using the procedures described in example 31.

ES/MS m/z: 434.2 (neg, M−H); ¹H NMR (500 MHz, acetone-d₆) δ 8.35 (d, 1H), 7.65 (d, 1H), 6.92 (d, 1H), 6.89 (dd, 1H), 6.84 (d, 1H), 6.64 (dd, 1H), 3.95 (br s, 2H).

### Example 56

### 2-[6-Hydroxy-2-(4-hydroxy-phenyl)-4-methyl-3H-inden-1-yl]-furan-3-carbonitrile (E56)

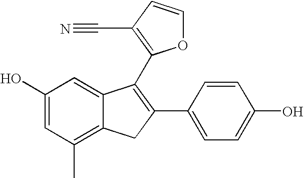

The title compound was synthesized using the procedures described in example 31.

ES/MS m/z: 330.2 (pos, M+H); ¹H NMR (500 MHz, acetone-d₆) δ 7.76 (d, 1H), 7.03-7.10 (m, 2H), 6.83 (d, 1H), 6.67-6.72 (m, 2H), 6.50 (d, 1H), 6.46 (d, 1H), 3.73 (s, 2H), 2.21 (s, 3H).

### Example 57

### 2-[5-Bromo-6-hydroxy-2-(4-hydroxy-phenyl)₄-methyl-3H-inden-1-yl]-furan-3-carbonitrile (E57)

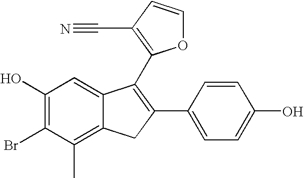

The title compound was synthesized using the procedures described in example 31.

ES/MS m/z: 408.5 (pos, M+H); ¹H NMR (500 MHz, acetone-d₆) δ 7.92 (d, 1H), 7.21-7.26 (m, 2H), 7.00 (d, 1H), 6.83-6.88 (m, 3H), 4.00 (s, 2H), 2.49 (s, 3H).

### Example 58

### 2-(4-Hydroxy-phenyl)-3-phenyl-7-trifluoromethyl-1H-inden-5-ol (E58)

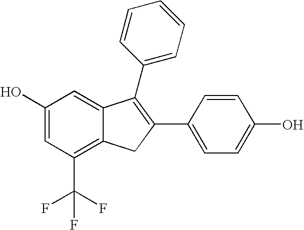

The title compound was synthesized using the procedures described in example 31.

ES/MS m/z: 369.2 (pos, M+H), 367.2 (neg, M−H); ¹H-NMR (500 MHz, acetone-d₆) δ 7.52−7.47 (m, 2H), 7.44−7.40 (m, 1H), 7.36−7.33 (m, 2H), 7.24−7.20 (m, 2H), 7.96−7.93 (d, 1H), 6.79−6.77 (d, 1H), 6.73−6.70 (m, 2H), 4.00−3.98 (m, 2H).

### Example 59

### 2-(2,6-Difluoro-4-hydroxy-phenyl)-7-methyl-3-phenyl-1H-inden-5-ol (E59)

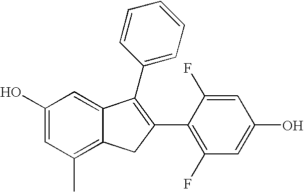

The title compound was synthesized using the procedures described in example 31.

ES/MS m/z: 351.2 (pos, M+H), 349.3 (neg, M−H); ¹H-NMR (500 MHz, MeOD) δ 7.32 (m, 2H), 7.28−7.23 (m, 3H), 6.64 (d, 1H), 6.55 (d, 1H), 6.32−6.27 (m, 2H), 3.57 (s, 2H), 2.33 (s, 3H).

### Example 60

### 2-(2,5-Difluoro-4-hydroxy-phenyl)-7-methyl-3-phenyl-1H-inden-5-ol (E60)

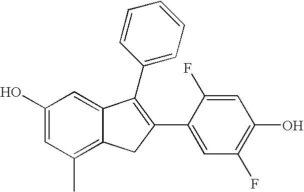

The title compound was synthesized using the procedures described in example 31.

ES/MS m/z: 351.3 (pos, M+H), 349.3 (neg, M−H); ¹H-NMR (500 MHz, MeOD) δ 7.39 (m, 2H), 7.33 (r, 1H), 7.27 (m, 2H), 6.73 (dd, 1H), 6.60 (dd, 1H), 6.56 (d, 1H), 6.53 (d, 1H), 3.67 (s, 2H), 2.34 (s, 3H).

### Example 61

### 2-(4-Hydroxy-3-trifluoromethyl-phenyl)-7-methyl-3-phenyl-1H-inden-5-ol (E61)

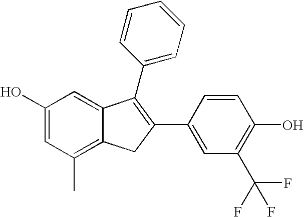

The title compound was synthesized using the procedures described in example 31.

ES/MS m/z: 383.3 (pos, M+H), 381.3 (neg, M−H); ¹H-NMR (500 MHz, MeOD) δ 7.45 (m, 2H), 7.38 (m, 2H), 7.32 (dd, 1H), 7.30 (m, 2H), 6.75 (d, 1H), 6.50 (d, 1H), 6.40 (d, 1H), 3.70 (s, 2H), 2.36 (s, 3H).

### Example 62

### 2-(4-Hydroxy-2,6-dimethyl-phenyl)-7-methyl-3-phenyl-1H-inden-5-ol (E62)

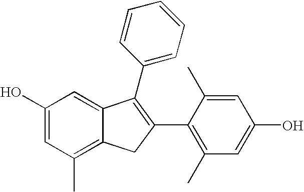

The title compound was synthesized using the procedures described in example 31.

ES/MS m/z: 343.4 (pos, M+H), 341.3 (neg, M−H); ¹H-NMR (500 MHz, MeOD) δ 7.25 (m, 2H), 7.22−7.18 (m, 3H), 6.71 (d, 1H), 6.53 (d, 1H), 6.44 (s, 2H), 3.43 (s, 2H), 2.32 (s, 3H), 2.01 (s, 6H).

### Example 63

### 2-(3,5-Dichloro hydroxy-phenyl)-7-methyl-3-phenyl-1H-inden-5-ol (E63)

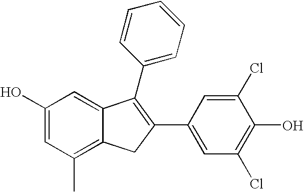

The title compound was synthesized using the procedures described in example 31.

ES/MS m/z: 383.2 (pos, M+H), 381.3 (neg, M−H); ¹H-NMR (500 MHz, MeOD) δ 7.47 (m, 2H), 7.41 (m, 1H), 7.29 (m, 2H), 7.15 (s, 2H), 6.51 (d, 1H), 6.39 (d, 1H), 3.67 (s, 2H), 2.35 (s, 3H).

### Example 64

### 2-(4-Hydroxy-phenyl)-7-methyl-3-thiazol-2-yl-1H-inden-5-ol (E64)

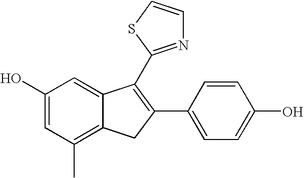

The title compound was synthesized using the procedures described in example 31.

ES/MS m/z: 322.3 (pos, M+H), 320.3 (neg, M−H); ¹H NMR (500 MHz, acetone-d₆) δ 7.93 (d, 1H), 7.58 (d, 1H), 7.28 (m, br, 2H), 7.27 (s, 1H), 6.87 (s, 1H), 6.85 (s, 1H), 6.59 (d, 1H), 3.73 (d, 2H), 2.32 (s, 3H).

### Example 65

### 1-{3-[6-Hydroxy-2-(4-hydroxy-phenyl)-4-methyl-3H-inden-1-yl]-furan-2-yl}-pentan-1-one (E65)

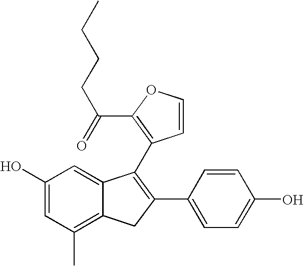

The title compound was synthesized using the procedures described in example 31.

ES/MS m/z: 389.2 (pos, M+H); ¹H NMR (500 MHz, CDCl₃) δ 7.68 (d, 2H), 6.72 (dd, 2H), 6.53 (d, 1H), 6.46 (d, 1H), 6.30 (d, 1H) 3.75 (br d, 2H), 2.58 (t, 2H), 2.37 (s, 3H), 1.13 (q, br, 2H), 0.88 (t, b, 2H), 0.72 (t, 3H). (One aromatic peak is hidden beneath the CDCl₃-signal)

### Example 66

### 4-[2-(3-Fluoro-4-hydroxy-phenyl)-6-hydroxy-4-methyl-3H-inden-1-yl]-thiophene-3-carbonitrile (E66)

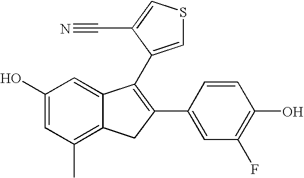

The title compound was synthesized using the procedures described in example 31.

ES/MS m/z: 364.2 (pos, M+H), 362.2 (neg, M−H); ¹H-NMR (500 MHz, MeOH-d₄) δ 8.40 (d, 1H), 7.61 (d, 1H), 6.95-6.91 (m, 2H), 6.79 (t, 1H), 6.52 (d, 1H), 6.37 (d, 1H), 3.89 (d, 1H), 3.62 (d, 1H) and 2.36 (s, 3H).

### Example 67

### 2-[2-(3-Fluoro-4-hydroxy-phenyl)-6-hydroxy-4-methyl-3H-inden-1-yl]-thiophene-3-carbonitrile (E67)

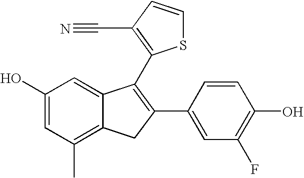

The title compound was synthesized using the procedures described in example 31.

ES/MS m/z: 364.2 (pos, M+H), 362.2 (neg, M−H); ¹H-NMR (500 MHz, MeOH-d₄) δ 7.75 (d, 1H), 7.42 (d, 1H), 6.98-6.93 (m, 2H), 6.82 (t, 1H), 6.55 (d, 1H), 6.45 (d, 1H), 3.81 (s, 2H) and 2.37 (s, 3H).

### Example 68

### 4-[2-(2,5-Difluoro-4-hydroxy-phenyl)-6-hydroxy-4-methyl-3H-inden-1-yl]-thiophene-3-carbonitrile (E68)

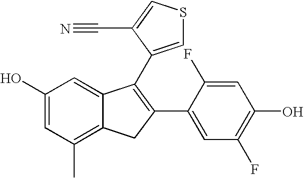

The title compound was synthesized using the procedures described in example 31.

ES/MS m/z: 382.2 (pos, M+H), 380.2 (neg, M−H); ¹H ¹H-NMR (500 MHz, MeOH-d₄) δ 8.32 (d, 1H), 7.58 (d, 1H), 6.79 (dd, 1H), 6.61 (dd, 1H), 6.56 (d, 1H), 6.51 (d, 1H), 3.75 (br s, 2H) and 2.35 (s, 3H).

### Example 69

### 2-[2-(2,5-Difluoro-4-hydroxy-phenyl)-6-hydroxy-4-methyl-3H-inden-1-yl]-thiophene-3-carbonitrile (E69)

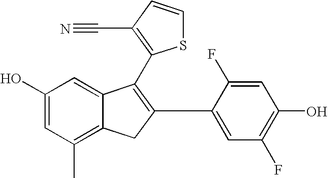

The title compound was synthesized using the procedures described in example 31. ES/MS m/z: 382.2 (pos, M+H), 380.2 (neg, M−H); ¹H ¹H-NMR (500 MHz, MeOH-d₄) δ 7.69 (d, 1H), 7.35 (d, 1H), 6.87 (dd, 1H), 6.64 (d, 1H), 6.63 (dd, 1H), 6.59 (d, 1H), 3.80 (s, 2H) and 2.36 (s, 3H).

### Example 70

### 4-Fluoro-2-(4-hydroxy-phenyl)-3-phenyl-1H-inden-5-ol (E70)

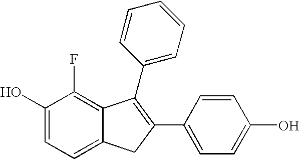

The title compound was synthesized using the procedures described in example 31.

ES/MS m/z: 319.1 (pos, M+H), 317.2 (neg, M−H); ¹H-NMR (500 MHz, CDCl₃) δ 7.44−7.39 (m, 2H), 7.38−7.34 (m, 1H), 7.34−7.30 (m, 2H), 7.24−7.20 (d, 1H), 7.15−7.11 (m, 2H), 6.82−6.79 (d, 1H), 6.69−6.65 (m, 2H), 4.96−4.94 (d, OH), 4.64 (s, OH), 3.80 (s, 2H).

### Example 71

### 4-Fluoro-2-(3-fluoro-4-hydroxy-phenyl)-3-phenyl-1H-inden-5-ol (E71)

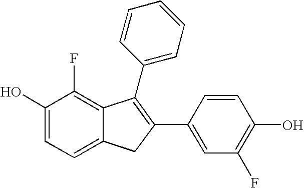

The title compound was synthesized using the procedures described in example 31.

ES/MS m/z: 337.4 (pos, M+H), 335.5 (neg, M−H); ¹H-NMR (500 MHz, CDCl₃) δ 7.46−7.41 (m, 2H), 7.41−7.36 (m, 1H), 7.33−7.29 (m, 2H), 7.24−7.21 (d, 1H), 6.96−6.91 (m, 2H), 6.85−6.81 (t, 1H), 6.81−6.78 (d, 1H), 5.03−5.01 (d, 1H), 4.98−4.95 (d, 1H), 3.78 (s, 2H).

### Example 72

### 2-(2,3-Difluoro-4-hydroxy-phenyl)-4-fluoro-3-phenyl-1H-inden-5-ol (E72)

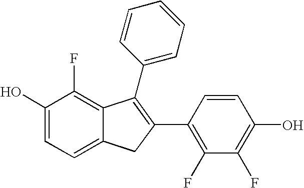

The title compound was synthesized using the procedures described in example 31.

ES/MS m/z: 355.4 (pos, M+H), 353.2 (neg, M−H); ¹H-NMR (500 MHz, CDCl₃) δ 7.39−7.35 (m, 2H), 7.35−7.30 (m, 1H), 7.29−7.27 (m, 2H), 7.27−7.23 (d, 1H), 6.99−6.96 (d, 1H), 6.70−6.65 (m, 1H), 6.59−6.55 (m, 1H), 5.16−5.13 (d, OH), 5.02−4.99 (d, OH), 3.82 (s, 2H).

### Example 73

### 4,6-Difluoro-2-(4-hydroxy-phenyl)-3-phenyl-1H-inden-5-ol (E73)

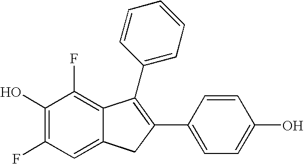

The title compound was synthesized using the procedures described in example 31.

ES/MS m/z: 337.4 (pos, M+H), 335.5 (neg, M−H); ¹H-NMR (500 MHz, acetone-d₆) δ 8.44 (s, OH), 7.45−7.32 (m, 5H), 7.17−7.13 (m, 1H), 7.10−7.05 (m, 2H), 6.68−6.64 (m, 2H), 3.90 (s, 2H).

### Example 74

### 4,6-Difluoro-2-(3-fluoro-4-hydroxy-phenyl)-3-phenyl-1H-inden-5-ol (E74)

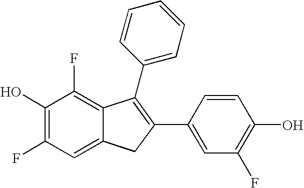

The title compound was synthesized using the procedures described in example 31.

ES/MS m/z: 355.4 (pos, M+H), 353.2 (neg, M−H); ¹H-NMR (500 MHz, acetone-d₆) δ 7.45−7.41 (m, 2H), 7.41−7.38 (m, 1H), 7.38−7.34 (m, 2H), 7.18−7.14 (m, 1H), 6.94−6.87 (m, 2H), 6.85−6.80 (m, 1H), 3.93 (s, 2H).

### Example 75

### 2-(2,3-Difluoro-4-hydroxy-phenyl)-4,6-difluoro-3-phenyl-1H-inden-5-ol (E75)

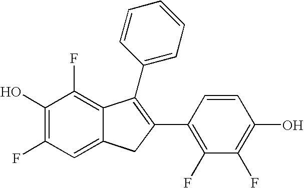

The title compound was synthesized using the procedures described in example 31.

ES/MS m/z: 373.1 (pos, M+H), 370.9 (neg, M−H); ¹H-NMR (500 MHz, acetone-d₆) δ 7.35−7.26 (m, 5H), 7.23−7.19 (m, 1H), 6.77−6.72 (m, 1H), 6.68-6.63 (m, 1H), 3.90 (s, 2H).

### Example 76

### 3-[7-Fluoro-6-hydroxy-2-(4-hydroxy-phenyl)-3H-inden-1-yl]-thiophene-2-carbonitrile (E76)

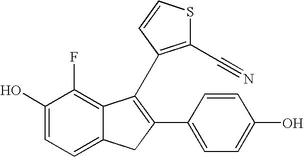

The title compound was synthesized using the procedures described in example 31.

ES/MS m/z: 350.3 (pos, M+H), 348.1 (neg, M−H); ¹H-NMR (500 MHz, acetone-d₆) δ 8.12−8.10 (d, 1H), 7.32−7.28 (m, 1H), 7.26−7.24 (d, 1H), 7.15−7.11 (m, 2H), 6.78−6.74 (m, 2H), 6.71−6.68 (d, 1H), 3.93 (s, 2H).

### Example 77

### 3-[7-Fluoro-2-(3-fluoro-4-hydroxy-phenyl)-6-hydroxy-3H-inden-1-yl]-thiophene-2-carbonitrile (E77)

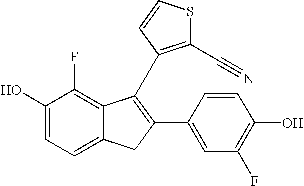

The title compound was synthesized using the procedures described in example 31.

ES/MS m/z: 368.0 (pos, M+H), 366.1 (neg, M−H); ¹H-NMR (500 MHz, acetone-d₆) δ 8.16−8.14 (d, 1H), 7.34−7.29 (m, 2H), 7.01−6.97 (m, 1H), 6.96−6.89 (m, 2H), 6.73−6.70 (d, 1H), 3.95 (s, 2H).

### Example 78

### 3-[2-(2,3-Difluoro-4-hydroxy-phenyl)-7-fluoro-6-hydroxy-3H-inden-1-yl]-thiophene-2-carbonitrile (E78)

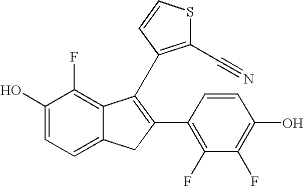

The title compound was synthesized using the procedures described in example 31.

ES/MS m/z: 386.3 (pos, M+H), 384.1 (neg, M−H); ¹H-NMR (500 MHz, acetone-d₆) δ 8.08−8.06 (d, 1H), 7.38−7.34 (m, 1H), 7.33−7.31 (d, 1H), 6.89−6.86 (d, 1H), 6.84−6.79 (m, 1H), 6.79−6.74 (m, 1H), 3.94 (s, 2H).

### Example 79

### 4-[2-(2,3-Difluoro-4-hydroxy-phenyl)-6-hydroxy-3H-inden-1-yl]-thiophene-3-carbonitrile (E79)

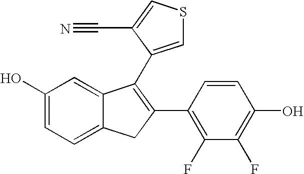

The title compound was synthesized using the procedures described in example 31.

ES/MS m/z: 368.0 (pos, M+H), 366.1 (neg, M−H); ¹H NMR (500 MHz, acetone-d₆) δ 8.37 (d, 1H), 7.67 (d, 1H), 7.33 (d, 1H), 6.76 (m, 2H), 6.69 (m, 2H), 3.88 (d, br, 2H)

### Example 80

### 4-[6-Hydroxy-2-(4-hydroxy-phenyl)-3H-inden-1-yl]-thiophene-3-carbonitrile (E80)

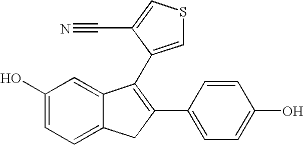

The title compound was synthesized using the procedures described in example 31.

ES/MS m/z: 332.3 (pos, M+H); ¹H NMR (500 MHz, acetone-d₆) δ 8.37 (d, 1H), 7.56 (d, 1H), 7.19 (d, 1H), 7.04 (dd, 2H), 6.62 (dd, 2H), 6.58 (dd, 1H), 6.41 (d, 1H), 3.75 (d, broad, 2H)

### Example 81

### 2-(4-Hydroxy-phenyl)-3-(2-isobutyl-2H-pyrazol-3-yl)-7-methyl-1H-inden-5-ol (E81)

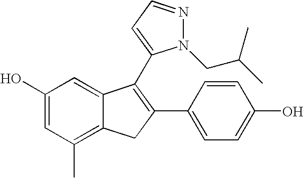

The title compound was synthesized according to the methods outlined in Scheme 9 and Scheme 8.

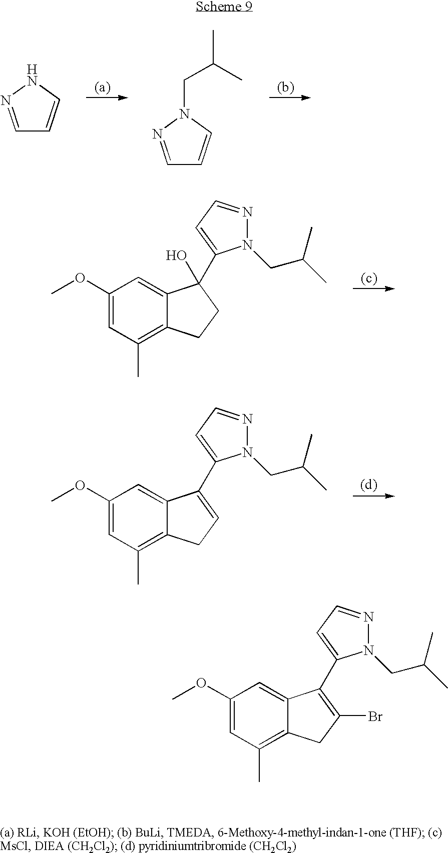

Step (a): 8.81 mmol of pyrazole, 8.81 mmol of 1-iodo-2-methylpropane, and 10.58 mmol of KOH were mixed with 2 mL of 95% ethanol in a 5 ml-microwave vessel. The mixture was heated to 130° C. for 30 minutes and then cooled to room temperature, poured into water and extracted with dichloromethane. The mixture was filtered through an SPE-phase separator. The organic phase was carefully evaporated and the residue was filtered through a silica plug (eluted with dichloromethane) to give 1.10 mmol of the desired product.

Step (b): To a mixture of 1.82 mmol BuLi (2.5 M in hexanes) and 1.82 mmol TMEDA in THF (0.5 mL) at −78° C. was added dropwise 1.82 mmol of N-isobutylpyrazole. The mixture was allowed to attain room temperature and stirred for 15 minutes. 6-Methoxy-4-methyl-indan-1-one (0.45 mmol) was added and the reaction was stirred at room temperature for 2 h. The reaction was quenched with water, extracted with dichloromethane and the mixture was filtered through a phase separator. The organic phase was evaporated and the residue was purified by column chromatography (mobile phase dichloromethane-MeOH 99:1->9:1) to give 0.080 mmol of the desired product and a considerable amount of unreacted 6-Methoxy-4-methyl-indan-1-one.

Step (c), (d): A mixture of 0.080 mmol of 1-(2-Isobutyl-2H-pyrazol-3-yl)-6-methoxy-4-methyl-indan-1-ol, 0.16 mmol of methylsulfonic chloride, and 0.16 mmol of diisopropylethylamine in 3 mL of CH₂Cl₂ was refluxed for 30 minutes, cooled, washed with NaHCO₃ (saq) and filtered through a phase separator. The organic phase containing the intermediate 1-Isobutyl-5-(6-methoxy-4-methyl-3H-inden-1-yl)-1H-pyrazole was cooled in an ice bath under stirring. 0.08 mmol of Pyridinium perbromide was added and the mixture was stirred at 0° C. for 10 minutes. Water was added and the mixture was filtered through a phase separator. The organic phase was placed on a short silica column and the product was eluted with a gradient ranging from pure dichloromethane to a 9:1 mixture of dichloromethane and methanol. Yield: 0.017 mmol. This intermediate was then used to synthesize example 81 according to steps (d) and (e) Scheme 8.

ES/MS m/z: 361.3 (pos., M+H), 359.4 (neg., M−H). ¹H-NMR (500 MHz, MeOH-d₄): 7.67 (d, 1H), 7.12 (d, 2H), 6.68 (d, 2H), 6.51−6.49 (m, 1H), 6.43 (d, 1H), 6.36 (d, 1H), 4.03−3.97 (m, 1H), 3.64−3.55 (m, 1H), 3.50−3.46 (m, 2H), 1.93−1.84 (m, 2H), 0.64 (d, 3H), 0.55 (d, 3H).

### Example 82

### 3-(1-Ethyl-1H-pyrrol-2-yl)-2-(4-hydroxy-phenyl)-7-methyl-1H-inden-5-ol (E82)

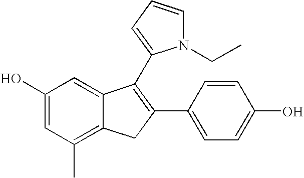

The title compound was synthesized using the procedures described in example 81.

ES/MS m/z: 332.3 (pos., M+H), 330.3 (neg., M−H). ¹H-NMR (500 MHz, CDCl₃): 7.09 (d, 2H), 6.77−6.44 (m, 1H), 6.67 (d, 2H), 6.51−6.48 (m, 2H), 6.28−6.25 (m, 1H), 6.14−6.12 (m, 1H), 4.07 (dd, 1H), 3.85−3.77 (m, 1H), 3.60−3.38 (m, 2H), 2.32 (s, 3H), 0.99 (t, 3H).

### Example 83

### 2-(4-Hydroxy-phenyl)-7-methyl-3-(1-methyl-1H-imidazol-2-yl)-1H-inden-5-ol (E83)

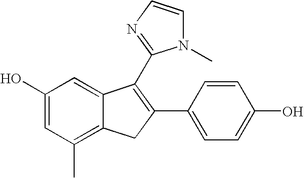

The title compound was synthesized using the procedures described in example 81.

ES/MS m/z: 319.3 (pos., M+H), 317.3 (neg., M−H). ¹H-NMR (500 MHz, MeOH-d₄): 7.18 (d, 1H), 7.17 (d, 1H), 7.02 (d, 2H), 6.69 (d, 2H), 6.51−6.49 (m, 1H), 6.40 (d, 1H), 4.10−3.51 (m, 2H), 3.23 (s, 3H), 2.36 (s, 3H).

### Example 84

### 2-(3-Fluoro-4-hydroxy-phenyl)-7-methyl-3-(2-methyl-2H-pyrazol-3-yl)-1H-inden-5-ol (E84)

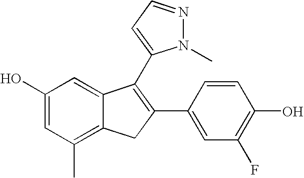

The title compound was synthesized using the procedures described in example 81.

ES/MS m/z: 337.3 (pos., M+H), 335.3 (neg., M−H). ¹H-NMR (500 MHz, MeOH-d₄): 7.66 (d, 1H), 6.93−6.87 (m, 2H), 6.85−6.79 (m, 1H), 6.52 (d, 1H), 6.41 (d, 1H), 6.34 (d, 1H), 3.94 (d, 1H), 3.63 (d, 1H), 3.44 (s, 3H), 2.36 (s, 3H).

### Example 85

### 2-(2,3-Difluoro-4-hydroxy-phenyl)-7-methyl-3-(2-methyl-2H-pyrazol-3-yl)-1H-inden-5-ol (E85)

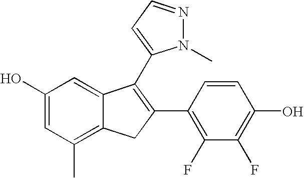

The title compound was synthesized using the procedures described in example 81.

ES/MS m/z: 355.3 (pos., M+H), 353.3 (neg., M−H). ¹H-NMR (500 MHz, MeOH-d₄): 7.56 (d, 1H), 6.73−6.67 (m, 1H), 6.63−6.58 (m, 1H), 6.58−6.55 (m, 1H), 6.45 (d, 1H), 6.32 (d, 1H), 4.00−3.60 (m, 2H), 3.48 (s, 3H), 2.36 (s, 3H).

### Example 86

### 2-(4-Hydroxy-phenyl)-7-methyl-3-(2-methyl-2H-pyrazol-3-yl)-1H-inden-5-ol (E86)

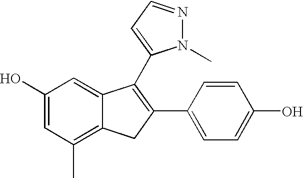

The title compound was synthesized using the procedures described in example 81.

ES/MS m/z: 319.3 (pos., M+H). ¹H-NMR (500 MHz, MeOH-d₄): 7.65 (d, 1H), 7.10 (d, 2H), 6.73 (d, 2H), 6.58 (d, 1H), 6.40−6.38 (m, 1H), 6.37 (d, 1H), 3.99−3.91 (m, 2H), 3.38 (s, 3H), 2.48 (s, 3H).

### Example 87

### 3-(2-Ethyl-2H-pyrazol-3-yl)-2-(4-hydroxy-phenyl)-7-methyl-1H-inden-5-ol (E87)

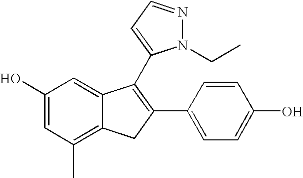

The title compound was synthesized using the procedures described in example 81.

ES/MS m/z: 333.3 (pos., M+H), 331.3 (neg., M−H). ¹H-NMR (500 MHz, MeOH-d₄): 7.67 (d, 1H), 7.13 (d, 2H), 6.68 (d, 2H), 6.48-6.52 (m, 1H), 6.37 (d, 1H), 6.29 (d, 1H), 3.99−3.91 (m, 1H), 3.84−3.71 (m, 2H), 6.68−6.60 (m, 1H), 2.36 (s, 3H), 1.07 (t, 3H).

### Example 88

### 7-Difluoromethyl-2-(4-hydroxy-phenyl)-3-phenyl-1H-inden-5-ol (E88)

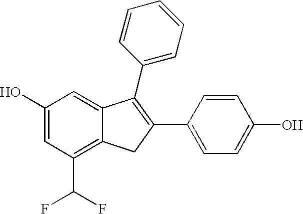

The title compound was synthesized according to the methods outlined in Scheme 10.

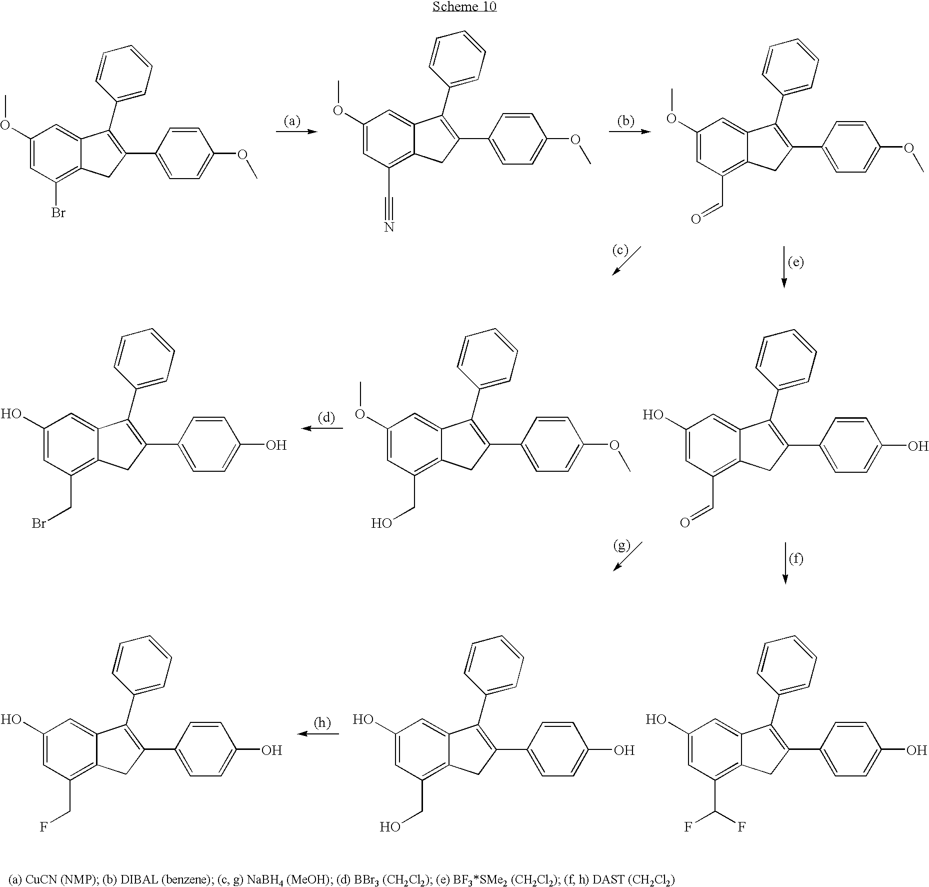

Step (a): 0.31 mmol of 7-Bromo-5-methoxy-2-(4-methoxy-phenyl)-3-phenyl-1H-indene, synthesized according to the methods outlined in Scheme 8, was dissolved in 1 mL 1-methyl-2-pyrrolidinone and 0.40 mmol of copper cyanide was added. The mixture was heated at 175° C. over night. Sat. NH₄Cl and ethyl acetate were added and phases separated. After flash chromatographic separation 0.29 mmol of 6-Methoxy-2-(4-methoxy-phenyl)-1-phenyl-3H-indene-4-carbonitrile were obtained.

Step (b): To 0.41 mmol of 6-Methoxy-2-(4-methoxy-phenyl)-1-phenyl-3H-indene-4-carbonitrile in 3 mL benzene, was slowly added 4.0 mmol of diisobutylaluminum hydride at 0° C. The mixture was allowed to warm up to room temperature and left over night. The mixture was then cooled to 0° C. and quenched with some 1M HCl, water and ethyl acetate/diethyl ether (1:1) were added and phases separated. After flash chromatographic separation 0.15 mmol of 6-Methoxy-2-(4-methoxy-phenyl)-1-phenyl-3H-indene-4-carbaldehyde were obtained.

Step (c, g): 0.07 mmol of 6-Methoxy-2-(4-methoxy-phenyl)-1-phenyl-3H-indene-4-carbaldehyde was dissolved in methanol and treated with 0.34 mmol sodium boron hydride for 20 minutes at 0° C. Mixture quenched with water and ethyl acetate/diethyl ether (1:1) were added and phases separated. After flash chromatographic separation 0.025 mmol of [6-Methoxy-2-(4-methoxy-phenyl)-1-phenyl-3H-inden-4-yl]-methanol

were obtained.

Step (d): 0.03 mmol of [6-Methoxy-2-(4-methoxy-phenyl)-1-phenyl-3H-inden-4-yl]-methanol was dissolved in 1.5 mL dichloromethane and treated with 0.13 mmol boron tribromide at 0° C. for 1 hour. The mixture was diluted with methanol and aqueous NaHCO₃, water was added and product extracted with dichloromethane. After flash chromatographic separation 0.02 mmol of 7-Bromomethyl-2-(4-hydroxy-phenyl)-3-phenyl-1H-inden-5-ol were obtained.

Step (e): To 0.056 mmol of 6-Methoxy-2-(4-methoxy-phenyl)-1-phenyl-3H-indene-4-carbaldehyde in 1 mL dichloromethane was added 1.40 mmol of boron trifluoride-sulfide and mixture stirred over night. Water and ethyl acetate were added and phases separated. After flash chromatographic separation 0.015 mmol of 6-Hydroxy-2-(4-hydroxy-phenyl)-1-phenyl-3H-indene-4-carbaldehyde were obtained.

Step (f, h): In 1 mL dichloromethane, were 0.015 mmol of 6-Hydroxy-2-(4-hydroxy-phenyl)-1-phenyl-3H-indene-4-carbaldehyde and 0.080 mmol of diethylaminosulphur trifluoride heated at 40° C. over night. The mixture was cooled to room temperature and water and ethyl acetate were added and phases separated. After flash chromatographic separation 0.003 mmol of 7-Difluoromethyl-2-(4-hydroxy-phenyl)-3-phenyl-1H-inden-5-ol were obtained.

ES/MS m/z: 349.3 (neg, M−H); ¹H-NMR (500 MHz, CDCl₃) δ 7.44-7.41 (m, 2H), 7.40−7.37 (m, 1H), 7.34−7.31 (m, 2H), 7.19−7.16 (m, 2H), 6.89−6.65 (t, 1H), 6.81−6.79 (m, 1H), 6.73−6.71 (m, 1H), 6.69−6.66 (m, 2H), 3.49 (s, 2H).

### Example 89

### 7-Fluoromethyl-2-(4-hydroxy-phenyl)-3-phenyl-1H-inden-5-ol (E89)

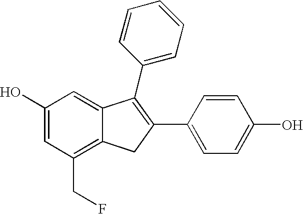

The title compound was synthesized according to the methods outlined in Scheme 10.

ES/MS m/z: 333.5 (pos, M+H), 331.3 (neg, M−H); ¹H-NMR (500 MHz, acetone-d₆) δ 8.41 (s, OH), 8.17 (s, OH), 7.50−7.45 (m, 2H), 7.43−7.37 (m, 1H), 7.36−7.31 (m, 2H), 7.22−7.16 (m, 2H), 6.77−6.73 (m, 1H), 6.72−6.67 (m, 2H), 6.61−6.57 (m, 1H), 5.59 (s, 1H), 5.49 (s, 1H), 3.88−3.84 (d, 2H).

### Example 90

### 7-Bromomethyl-2-(4-hydroxy-phenyl)-3-phenyl-1H-inden-5-ol (E90)

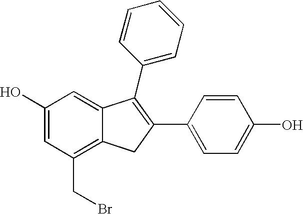

The title compound was synthesized according to the methods outlined in Scheme 10.

ES/MS m/z: 393.2 and 395.0 (pos, M+H); ¹H-NMR (500 MHz, acetone-d₆) δ 8.43 (s, OH), 8.18 (s, OH), 7.50−7.45 (m, 2H), 7.42−7.37 (m, 1H), 7.35−7.31 (m, 2H), 7.23−7.19 (m, 2H), 6.79−6.77 (d, 1H), 6.73−6.68 (m, 2H), 6.57−6.55 (d, 1H), 4.75 (s, 2H), 3.92 (s, 2H).

### Example 91

### 2-(4-Hydroxy-phenyl)-3-phenyl-7-vinyl-1H-inden-5-ol (E91)

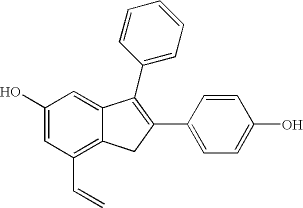

The title compound was synthesized according to the methods outlined in Scheme 11.

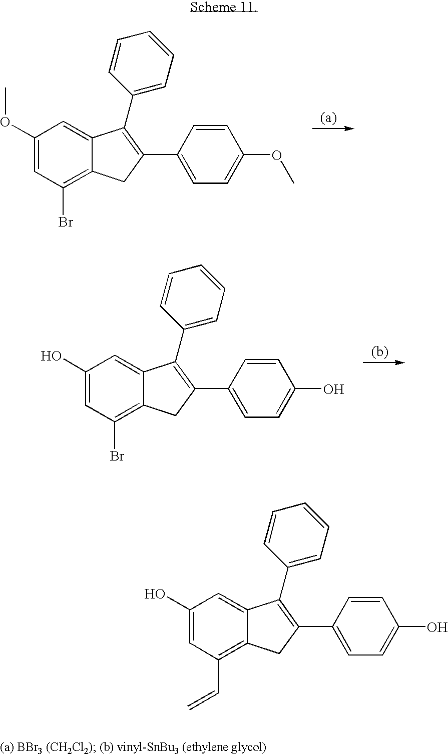

Step (a): To 0.15 mmol of 7-Bromo-5-methoxy-2-(4-methoxy-phenyl)-3-phenyl-1H-indene, synthesized according to the methods outlined in Scheme 8, in 3 mL dichloromethane was added 1.50 mmol boron tribromide at 0° C. and mixture was stirred for 2 hours. Water was added and phases separated. After flash chromatographic separation 0.13 mmol of 7-Bromo-2-(4-hydroxy-phenyl)-3-phenyl-1H-inden-5-ol were obtained.

Step (b): A mixture of 0.026 mmol 7-Bromo-2-(4-hydroxy-phenyl)-3-phenyl-1H-inden-5-ol, 0.040 mmol tributyl(vinyl)tin, 0.005 mmol tri(o-tolyl)phosphine and 0.003 palladium(II) acetate in 1 mL ethylene glycol were heated at 120° C. for 48 hours. The mixture was cooled and treated with activated carbon and filtered through MgSO₄. After flash chromatographic separation, reversed phase, 0.005 mmol of 2-(4-Hydroxy-phenyl)-3-phenyl-7-vinyl-1H-inden-5-ol were obtained.

ES/MS m/z: 327.3 (pos, M+H), 325.3 (neg, M−H); ¹H-NMR (500 MHz, acetone-d₆) δ 8.43 (s, OH), 8.04 (s, OH), 7.50−7.44 (m, 2H), 7.42−7.36 (m, 1H), 7.35−7.30 (m, 2H), 7.24−7.18 (m, 2H), 6.99−6.91 (dd, 1H), 6.89−6.87 (d, 1H), 6.72−6.68 (m, 2H), 6.55−6.52 (d, 1H), 5.87−5.80 (dd, 1H), 5.39−5.34 (dd, 1H), 3.88 (s, 2H).

### Example 92

### 4-[2-(3-Fluoro-4-methoxy-phenyl)-6-methoxy-4-methyl-3H-inden-1-yl]-thiophene-3-carbonitrile (E92)

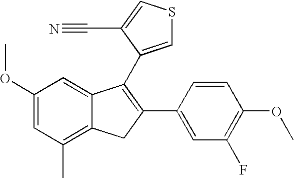

The title compound was synthesized using the procedures described in example 31.

EI/MS m/z: 392.3 (pos, M+H); ¹H-NMR (500 MHz, CDCl₃) δ 8.06−8.03 (d, 1H), 7.44−7.41 (d, 1H), 7.03−6.99 (m, 1H), 6.95−6.90 (m, 1H), 6.90−6.84 (t, 1H), 6.66−6.64 (m, 1H), 6.55−6.53 (d, 1H), 3.88 (s, 3H), 3.79 (s, 3H), 3.79 (s, 2H), 2.40 (s, 3H).

### Example 93

### 4-[4-Fluoro-6-hydroxy-2-(4-hydroxy-phenyl)-3H-inden-1-yl]-thiophene-3-carbonitrile (E93)

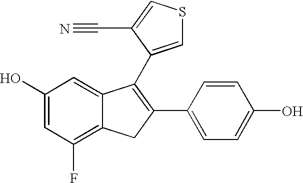

The title compound was synthesized using the procedures described in example 31.

ES/MS m/z: 348.2 (neg, M−H); ¹H NMR (500 MHz, d⁶-acetone) δ 8.38 (d, 1H), 7.59 (d, 1H), 7.04-7.09 (m, 2H), 6.61-6.66 (m, 2H), 6.33 (dd, 1H), 6.27 (d, 1H), 3.63-3.97 (br m, 2H).

### Example 94

### 4-[2-(2,3-Difluoro-4-hydroxy-phenyl)-fluoro-6-hydroxy-3H-inden-1-yl]thiophene-3-carbonitrile (E94)

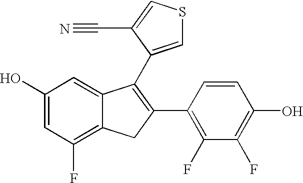

The title compound was synthesized using the procedures described in example 31.

ES/MS m/z: 384.2 (neg, M−H); ¹H NMR (500 MHz, d⁶-acetone) δ 8.39 (d, 1H), 7.69 (d, 1H), 6.78-6.84 (m, 1H), 6.68-6.73 (m, 1H), 6.56 (d, 1H), 6.50 (dd, 1H), 3.70-4.10 (br m, 2H).

### Example 95

### 4-[5,7-Difluoro-2-(3-fluoro-4-hydroxy-phenyl)-6-hydroxy-3H-inden-1-yl]-thiophene-3-carbonitrile (E95)

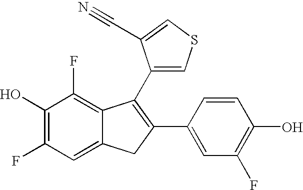

The title compound was synthesized using the procedures described in example 31.

EI/MS m/z: 386.3 (pos, M+H), 384.1 (neg, M−H); ¹H-NMR (500 MHz, acetone-d₆) δ 8.95−8.59 (br m, 2 OH), 8.51−8.48 (d, 1H), 7.68−7.65 (d, 1H), 7.21−7.16 (d, 1H), 6.99−6.87 (m, 3H), 4.04−3.96 (br m, 2H).

### Example 96

### 2-[4-Fluoro-6-hydroxy-2-(4-hydroxy-phenyl)-3H-inden-1-yl]-furan-3-carbonitrile (E96)

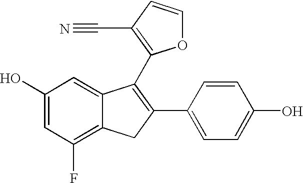

The title compound was synthesized using the procedures described in example 31.

ES/MS m/z: 334.4 (pos, M+H); ¹H NMR (500 MHz, d⁶-acetone) δ 7.78 (d, 1H), 7.06-7.12 (m, 2H), 6.85 (d, 1H), 6.69-6.74 (m, 2H), 6.53 (d, 1H), 6.38 (dd, 1H), 3.88 (s, 2H).

### Example 97

### 3-[6-Hydroxy-2-(4-hydroxy-phenyl)-4 methyl-3H-inden-1-yl]-furan-2-carbonitrile (E97)

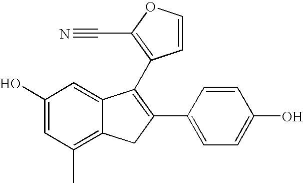

The title compound was synthesized using the procedures described in example 31.

ES/MS m/z: 329.4 (pos., M+H, neg., M−H); ¹H NMR (500 MHz, Acetone) δ 8.05 (d, 1H), 7.27-7.28 (dd, 2H), 6.80-6.81 (dd, 2H), 6.78 (d, 1H), 6.58 (d, 1H), 6.53 (d, 1H), 3.78 (s, 2H), 2.34 (s, 3H).

### Example 98

### 3-[2-(3-Fluoro-4-hydroxy-phenyl)-6-hydroxy-4-methyl-3H-inden-1-yl]-furan-2-carbonitrile (E98)

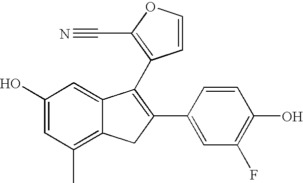

The title compound was synthesized using the procedures described in example 31.

ES/MS m/z: 347.3 (pos., M+H, neg., M−H); ¹H NMR (500 MHz, Acetone) δ 8.08 (d, 1H), 7.19 (d, 1H), 7.16 (d, 1H), 7.09 (dd, 1H), 7.08 (dd, 1H), 6.95-6.98 (t, 1H), 6.83 (d, 1H), 6.60 (dd, 1H), 6.55 (d, 1H), 3.81 (s, 2H), 2.35 (s, 3H).

### Example 99

### 3-[2-(2,3-Difluoro-4-hydroxy-phenyl)-6-hydroxy-4-methyl-3H-inden-1-yl]-furan-2-carbonitrile (E99)

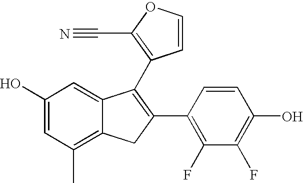

The title compound was synthesized using the procedures described in example 31.

ES/MS m/z: 365.3 (pos., M+H, neg., M−H); ¹H NMR (500 MHz, Acetone) δ 8.01 (d, 1H), 6.98−6.94 (t, 1H), 6.84 (d, 1H), 6.84−6.81 (t, 1H), 6.69 (d, 1H), 6.65 (d, 1H), 3.79 (s, 2H), 2.35 (s, 3H).

### Example 100

### 5-[6-Hydroxy-2-(4-hydroxy-phenyl)-4-trifluoromethyl-3H-inden-1-yl]-furan-2-carbonitrile (E100)

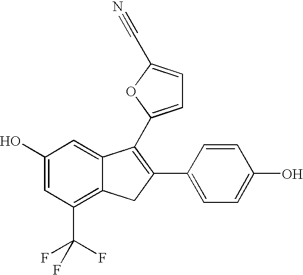

The title compound was synthesized using the procedures described in example 31.

EI/MS m/z: 384.2 (pos, M+H), 382.0 (neg, M−H); ¹H-NMR (500 MHz, acetone-d₆) δ 8.82 (br s, OH), 8.65 (br s, OH), 8.10−8.08 (d, 1H), 7.35−7.31 (m, 2H), 7.01−6.98 (d, 1H), 6.93−6.90 (d, 1H), 6.86−6.82 (m, 3H), 4.09−4.07 (m, 2H).

### Example 101

### 5-[2-(3-Fluoro-4-hydroxy-phenyl)-6-hydroxy-4-trifluoromethyl-3H-inden-1-yl]-furan-2-carbonitrile (E101)

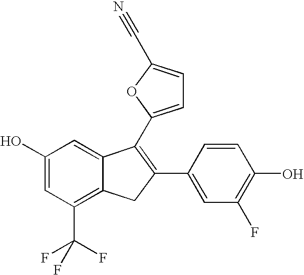

The title compound was synthesized using the procedures described in example 31.

EI/MS m/z: 402.2 (pos, M+H), 400.0 (neg, M−H); ¹H-NMR (500 MHz, acetone-d₆) δ 9.10−8.86 (br d, 2 OH), 7.57−7.55 (d, 1H), 7.38−7.36 (d, 1H), 7.29−7.24 (dd, 1H), 7.18−7.14 (m, 1H), 7.07−7.02 (t, 2H), 6.76−6.74 (d, 1H), 4.09−4.06 (m, 2H).

### Example 102

### 2-[6-Hydroxy-2-(4-hydroxy-phenyl)-4-trifluoromethyl-3H-inden-1-yl]-furan-3-carbonitrile (E102)

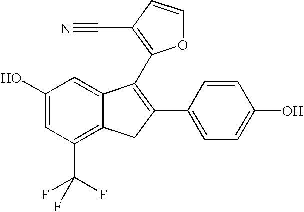

The title compound was synthesized using the procedures described in example 31.

EI/MS m/z: 384.2 (pos, M+H), 382.3 (neg, M−H); ¹H-NMR (500 MHz, acetone-d₆) δ 8.88 (br s, OH), 8.74 (br s, OH), 7.95−7.93 (d, 1H), 7.27−7.23 (m, 2H), 7.04−7.01 (m, 2H), 7.01−6.99 (d, 1H), 6.87−6.83 (m, 2H), 4.17−4.14 (m, 2H).

### Example 103

### 2-[2-(3-Fluoro-4-hydroxy-phenyl)-6-hydroxy-4-trifluoromethyl-3H-inden-1-yl]-furan-3-carbonitrile (E103)

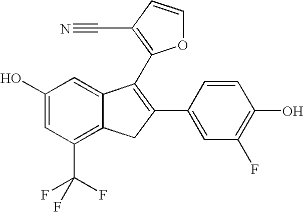

The title compound was synthesized using the procedures described in example 31.

EI/MS m/z: 402.2 (pos, M+H), 400.0 (neg, M−H); ¹H-NMR (500 MHz, acetone-d₆) δ 9.04 (br s, OH), 8.91 (br s, OH), 7.99−7.97 (d, 1H), 7.19−7.14 (dd, 1H), 7.09−7.07 (m, 2H), 7.06−7.03 (m, 1H), 7.03−6.00 (m, 2H), 4.20−4.16 (m, 2H).

### Example 104

### 3-[6-Hydroxy-2-(4-hydroxy-phenyl)-4-trifluoromethyl-3H-inden-1-yl]-furan-2-carbonitrile (E104)

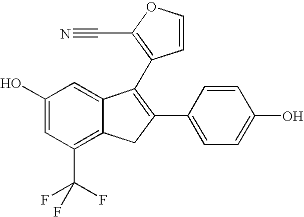

The title compound was synthesized using the procedures described in example 31.

EI/MS m/z: 384.2 (pos, M+H), 382.0 (neg, M−H); ¹H-NMR (500 MHz, acetone-d₆) δ 8.81 (br s, OH), 8.64 (br s, OH), 8.10−8.08 (d, 1H), 7.35−7.31 (m, 2H), 7.01−6.98 (m, 1H), 6.93−6.90 (m, 1H), 6.86−6.81 (m, 3H), 4.09−4.06 (m, 2H).

### Example 105

### 3-[2-(3-Fluoro-4-hydroxy-phenyl)-6-hydroxy-4-trifluoromethyl-3H-inden-1-yl]-furan-2-carbonitrile (E105)

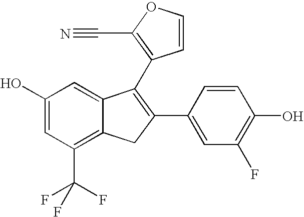

The title compound was synthesized using the procedures described in example 31.

EI/MS m/z: 402.2 (pos, M+H), 400.0 (neg, M−H); ¹H-NMR (500 MHz, acetone-d₆) δ 8.96−8.94 (br m, OH), 8.86−8.84 (br s, OH), 8.13−8.11 (d, 1H), 7.27−7.22 (dd, 1H), 7.16−7.12 (m, 1H), 7.04−7.01 (m, 1H), 7.01−6.96 (m, 1H), 7.95−7.93 (m, 1H), 6.90−6.88 (d, 1H), 4.12−4.09 (m, 2H).

### Example 106

### 3-[6-Hydroxy-2-(4-hydroxy-phenyl)-4-methyl-3H-inden-1-yl]-furan-2-carbaldehyde (E106)

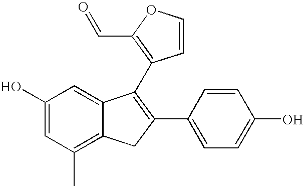

The title compound was synthesized using the procedures described in example 31.

EI/MS m/z: 333.5 (pos, M+H), 331.3 (neg, M−H); ¹H-NMR (500 MHz, acetone-d₆) δ 9.37−9.35 (s, 1H), 8.54−8.51 (s, OH), 8.09−8.06 (m, 1H), 8.04−8.01 (s, OH), 7.32−7.27 (d, 2H), 6.81−6.74 (m, 3H), 6.59−6.57 (m, 1H), 6.50−6.46 (m, 1H), 3.86−3.75 (br m, 2H), 2.36−2.33 (s, 3H).

### Example 107

### 4-[2-(2,4-Dihydroxy-phenyl)-6-hydroxy-4-methyl-3H-inden-1-yl]-thiophene-3-carbonitrile (E107)

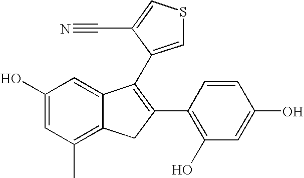

The title compound was synthesized using the procedures described in example 31.

ES/MS m/z: 361.4 (pos., M+H, neg., M−H); ¹H NMR (500 MHz, Acetone) δ 8.34 (d, 1H), 7.53 (d, 1H), 6.81−6.80 (d, 1H), 6.55 (d, 1H), 6.51 (d, 1H), 6.41 (d, 1H), 6.22 (d, 1H), 6.21 (dd, 1H), 3.64−3.61 (broad d, 2H), 2.31 (s, 3H).

### Example 108

### 7-Chloro-2-(4-hydroxy-phenyl)-3-phenyl-1H-inden-5-ol (E108)

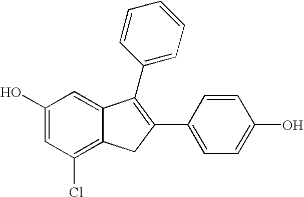

The title compound was synthesized using the procedures described in example 31.

EI/MS m/z: 335.6 (pos, M+H), 333.1 (neg, M−H); ¹H-NMR (500 MHz, acetone-d₆) δ 8.51−8.41 (br m, 2 OH), 7.50−7.45 (m, 2H), 7.43−7.38 (m, 1H), 7.35−7.30 (m, 2H), 7.23−7.18 (m, 2H), 6.73−6.68 (m, 3H), 6.55−6.52 (d, 1H), 3.83 (s, 2H).

### Example 109

### 4-[4-Chloro-2-(3-fluoro-4-hydroxy-phenyl)-6-hydroxy-3H-inden-1-yl]-thiophene-3-carbonitrile (E109)

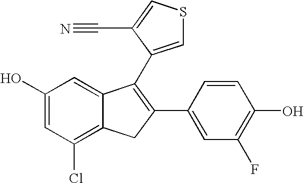

The title compound was synthesized using the procedures described in example 31.

EI/MS m/z: 384.2 (pos, M+H), 382.3 (neg, M−H); ¹H-NMR (500 MHz, acetone-d₆) δ 9.18−8.83 (br s, OH), 8.83−8.56 (br s, OH), 7.92−7.88 (d, 1H), 7.56−7.51 (d, 1H), 7.14−7.09 (dd, 1H), 7.08−7.03 (m, 1H), 6.99−6.93 (t, 1H), 6.81−6.79 (d, 1H), 6.64−6.62 (d, 1H), 4.00 (s, 2H).

### Example 110

### 2-[4-Chloro-6-hydroxy-2-(4-hydroxy-phenyl)-3H-inden-1-yl]-furan-3-carbonitrile (E110)

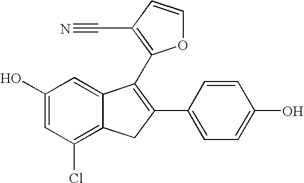

The title compound was synthesized using the procedures described in example 31.

EI/MS m/z: 350.3 (pos, M+H), 348.1 (neg, M−H); ¹H-NMR (500 MHz, acetone-d₆) δ 8.75-8.68 (br m, 2 OH), 7.93−7.91 (d, 1H), 7.25−7.21 (m, 2H), 7.00−6.98 (m, 1H), 6.87−6.83 (m, 2H), 6.80−6.76 (m, 2H), 3.98 (s, 2H).

### Example 111

### 2-[4-Chloro-2-(3-fluoro-4-hydroxy-phenyl)-6-hydroxy-3H-inden-1-yl]-furan-3-carbonitrile (E111)

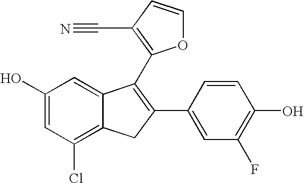

The title compound was synthesized using the procedures described in example 31.

EI/MS m/z: 368.0 (pos, M+H), 366.1 (neg, M−H); ¹H-NMR (500 MHz, acetone-d₆) δ 9.10−8.94 (br s, OH), 8.77−8.68 (br s, OH), 7.96−7.95 (d, 1H), 7.16−7.12 (m, 1H), 7.07−7.04 (m, 1H), 7.03−6.98 (m, 2H), 6.81 (s, 2H), 4.01 (s, 2H).

### Example 112

### 2-[2-(2,3-Difluoro-4-hydroxy-phenyl)-6-hydroxy-4-methyl-3H-inden-1-yl]-furan-3-carbonitrile (E112)

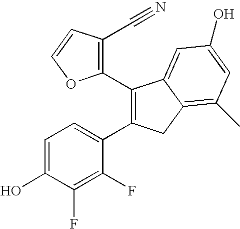

The title compound was synthesized using the procedures described in example 31.

¹HNMR (MeOD, 500 MHz): 7.77 (d, 1H); 6.88 (m, 1H); 6.77 (d, 1H); 6.74 (m, 2H); 6.54 (s, 1H); 3.74 (s, 2H); 2.22 (s, 3H). LC-MS (ES−): 364.2 (ES+): 366.2

### Example 113

### 2-[2-(2,3-Difluoro-4-hydroxy-phenyl)-7-fluoro-6-hydroxy-3H-inden-1-yl]-furan-3-carbonitrile (E113)

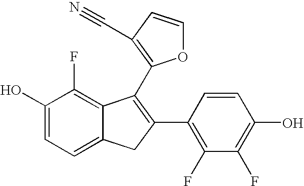

The title compound was synthesized using the procedures described in example 31.

ES/MS m/z: 368.1 (neg, M−H); ¹H NMR (500 MHz, d⁶-acetone) δ 7.92 (d, 1H), 7.37 (d, 1H), 7.18 (d, 1H), 6.99 (dt, 1H), 6.92 (d, 1H), 6.87 (dt, 1H), 4.00 (s, 2H).

### Example 114

### 2-[2-(3-Fluoro-4-hydroxy-phenyl)-7-fluoro-6-hydroxy-3H-inden-1-yl]-furan-3-carbonitrile (E114)

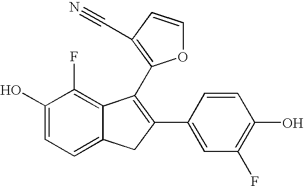

The title compound was synthesized using the procedures described in example 31.

ES/MS m/z: 350.1 (neg, M−H); ¹H NMR (500 MHz, d⁶-acetone) δ 7.96 (d, 1H), 7.34 (d, 1H), 7.08 (dd, 1H), 6.98-7.03 (m, 4H), 4.02 (s, 2H).

### Example 115

### 2-[2-(3-Chloro-5-fluoro-4-hydroxy-phenyl)-7-fluoro-6-hydroxy-3H-inden-1-yl]-furan-3-carbonitrile (E115)

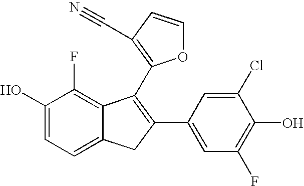

The title compound was synthesized using the procedures described in example 31.

ES/MS m/z: 384.0 (neg. M−H); ¹H NMR (500 MHz, d⁶-acetone) δ 7.99 (d, 1H), 7.36 (d, 1H), 7.16-7.20 (m, 1H), 7.04-7.11 (m, 2H), 7.02 (d, 1H), 4.05 (s, 2H).

### Example 116

### 2-[2-(2,3-Difluoro-4-hydroxy-phenyl)-4-fluoro-6-hydroxy-3H-inden-1-yl]-furan-3-carbonitrile (E116)

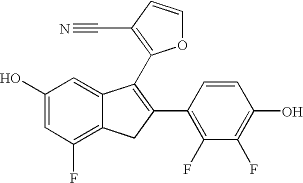

The title compound was synthesized using the procedures described in example 31.

EI/MS m/z: 370.1 (pos, M+H), 368.2 (neg, M−H); ¹H-NMR (500 MHz, acetone-d₆) δ 7.93−7.91 (d, 1H), 7.08−7.03 (m, 1H), 6.93−6.91 (d, 1H), 6.90−6.85 (m, 2H), 6.60−6.56 (dd, 1H), 4.02 (s, 2H).

### Example 117

### 2-[6-Hydroxy-2-(4-hydroxy-phenyl)-4-methyl-3H-inden-1-yl]-1-methyl-1H-imidazole-4-carbonitrile (E117)

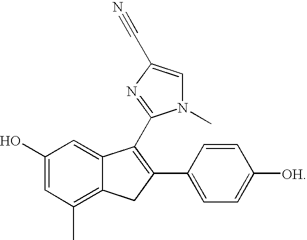

The title compound was synthesized using the procedures described in example 31.

ES/MS m/z: 342.1 (neg, M−H), 344.1 (pos, M+H); ¹H NMR (500 MHz, d⁶-acetone) δ 7.21 (s, 1H); 7.13 (m, 2H); 6.82 (m, 2H); 6.56 (m, 2H); 3.85 (br s, 2H); 3.29 (s, 3H); 2.34 (s, 3H).

### Example 118

### 2-[4-Chloro-2-(2,3-difluoro-4-hydroxy-phenyl)-6-hydroxy-3H-inden-1-yl]-furan-3-carbonitrile (E118)

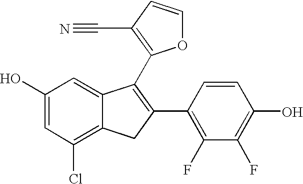

The title compound was synthesized using the procedures described in example 31.

EI/MS m/z: 386.3/388.4 (pos, M+H), 384.1/386.2 (neg, M−H); ¹H-NMR (500 MHz, acetone-d₆) δ 7.93−7.91 (d, 1H), 7.10−7.05 (m, 1H), 7.02−6.99 (d, 1H), 6.93−6.91 (d, 1H), 6.91−6:86 (m, 1H), 6.87−6.85 (d, 1H), 3.98 (s, 2H).

### Example 119

### 2-[2-(2,3-Difluoro-4-hydroxy-phenyl)-6-hydroxy-4-trifluoromethyl-3H-inden-1-yl]-furan-3-carbonitrile (E119)

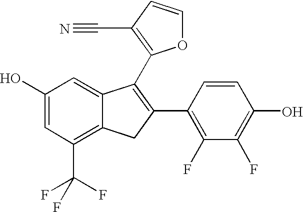

The title compound was synthesized using the procedures described in example 31.

EI/MS m/z: 420.2 (pos, M+H), 418.4 (neg, M−H); ¹H-NMR (500 MHz, acetone-d₆) δ 7.95−7.93 (d, 1H), 7.28−7.25 (m, 1H), 7.11−7.06 (m, 2H), 6.94−6.92 (d, 1H), 6.91−6.86 (m, 1H), 4.17−4.11 (br m, 2H).

### Example 120

### 2-[6-Hydroxy-2-(4-hydroxy-phenyl)-3,4-dimethyl-3H-inden-1-yl]-furan-3-carbonitrile (E120)

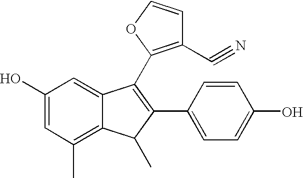

The title compound was synthesized using the procedures described in example 31.

ES/MS m/z: 342.1 (neg, M−H) ¹H NMR (500 MHz, d⁶-acetone) δ 7.86 (m, 1H); 7.13 (m, 2H); 6.91 (m, 1H); 6.85 (m, 2H); 6.64 (m, 1H); 4.19 (ddd, J=3.1, 6.9, 9.8 Hz, 1H); 3.42 (d, J=3.1, 3H); 1.22 (dd, J=3.1, 7.2, 3H).

### Example 121

### 2-[2-(3-Chloro-5-fluoro-4-hydroxy-phenyl)-6-hydroxy-4-methyl-3H-inden-1-yl]-furan-3-carbonitrile (E121)

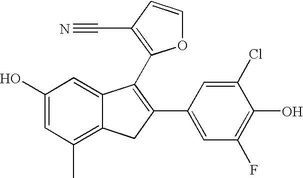

The title compound was synthesized using the procedures described in example 31.

ES/MS m/z: 380.03 (neg, M−H); ¹H NMR (500 MHz, d⁶-acetone) δ 7.97 (d, 1H); 7.19 (m, 1H); 7.07 (dd, 1H); 7.00 (d, 1H); 6.72 (d, 1H); 6.65 (d, 1H); 3.92 (s, 2H); 2.35 (s, 3H).

### Example 122

### 2-[6-Methoxy-2-(4-methoxy-phenyl)-4-methyl-3H-inden-1-yl]-furan-3-carbonitrile (E122)

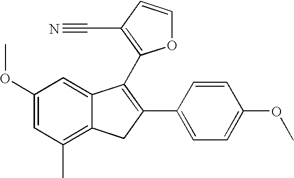

The title compound was synthesized using the procedures described in example 31.

ES/MS m/z: 357.4 (pos., M+H, neg., M−H); ¹H NMR (500 MHz, Acetone) δ 7.90 (d, 1H), 7.29−7.27 (broad d, 2H), 6.98 (d, 1H), 6.93−6.92 (broad d, 2H), 6.76 (d, 1H), 6.69 (d, 1H), 3.92 (s, 2H), 3.82 (s, 3H), 3.79 (s, 3H), 2.39 (s, 3H).

### Example 123

### 2-[7-Fluoro-6-hydroxy-2-(4-hydroxy-phenyl)-3H-inden-1-yl]-furan-3-carbonitrile (E123)

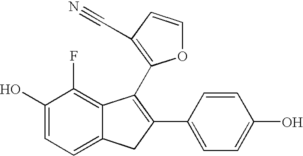

The title compound was synthesized using the procedures described in example 31.

ES/MS m/z: 332.1 (neg, M−H); ¹H NMR (500 MHz, d⁶-acetone) δ 7.92 (d, 1H), 7.32 (d, 1H), 7.15-7.21 (m, 2H), 6.98 (d, 1H), 6.95 (d, 1H), 6.81-6.86 (m, 2H), 3.99 (s, 2H).

### Example 124

### 2-[4-Chloro-2-(3-chloro-5-fluoro-4-hydroxy-phenyl)-6-hydroxy-3H-inden-1-yl]-furan-3-carbonitrile (E124)

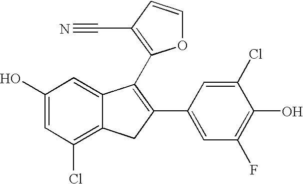

The title compound was synthesized using the procedures described in example 31.

EI/MS m/z: 402.2/404.6 (pos, M+H), 400.0/402.1 (neg, M−H); ¹H-NMR (500 MHz, acetone-d₆) δ 7.99−7.97 (d, 1H), 7.24−7.22 (m, 1H), 7.15−7.11 (dd, 1H), 7.03−7.01 (d, 1H), 6.88−6.83 (m, 1H), 4.05−4.01 (br m, 2H).

### Example 125

### 2-[4-Fluoro-2-(3-fluoro-4-hydroxy-phenyl)-6-hydroxy-3H-inden-1-yl]-furan-3-carbonitrile (E125)

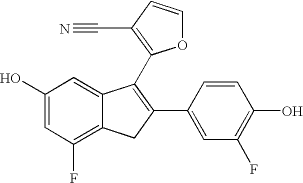

The title compound was synthesized using the procedures described in example 31.

EI/MS m/z: 352.4 (pos, M+H), 350.2 (neg, M−H); ¹H-NMR (500 MHz, acetone-d₆) δ 9.06−8.74 (br m, 2 OH), 7.96−7.94 (d, 1H), 7.14−7.09 (dd, 1H), 7.05−7.02 (m, 1H), 7.02−6.97 (m, 2H), 6.71−6.69 (d, 1H), 6.56−6.52 (dd, 1H), 4.04 (s, 2H).

### Example 126

### 2-[2-(3-Chloro-5-fluoro-4-hydroxy-phenyl)-4-fluoro-6-hydroxy-3H-inden-1-yl]-furan-3-carbonitrile (E126)

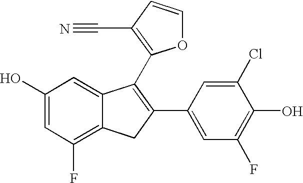

The title compound was synthesized using the procedures described in example 31.

EI/MS m/z: 386.3 (pos, M+H), 384.1 (neg, M−H); ¹H-NMR (500 MHz, acetone-d₆) δ 7.99−7.97 (d, 1H), 7.22−7.20 (m, 1H), 7.13−7.09 (dd, 1H), 7.02−7.00 (d, 1H), 6.76−6.74 (d, 1H), 6.58−6.55 (dd, 1H), 4.07 (s, 2H).

### Example 127

### 2-[2-(3-Chloro-5-fluoro-4-hydroxy-phenyl)-6-hydroxy-4-trifluoromethyl-3H-inden-1-yl]-furan-3-carbonitrile (E127)

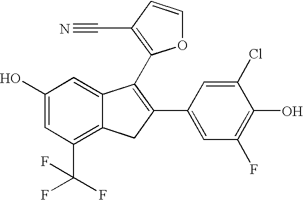

The title compound was synthesized using the procedures described in example 31.

EI/MS m/z: 436.4 (pos, M+H), 434.3 (neg, M−H); ¹H-NMR (500 MHz, acetone-d₆) δ 8.01−8.00 (d, 1H), 7.27−7.25 (m, 1H), 7.18−7.14 (m, 1H), 7.14−7.12 (m, 1H), 7.08−7.06 (m, 1H), 7.04−7.02 (d, 1H), 4.23−4.18 (br m, 2H).

Description of the Estrogen Receptor Binding Assay

The estrogen receptor ligand binding assays are designed as scintillation proximity assays (SPA), employing the use of tritiated estradiol (³H-E2) and recombinant expressed biotinylated estrogen receptor binding domains. The binding domains of human ERα (ERα-LBD, pET-N-AT #1, aa 301-595) and ERβ (ERβ-LBD, pET-N-AT #1, aa 255-530) proteins are produced in E. coli ((BL21, (DE3), pBirA)) at 22 C in 2×LB medium supplemented with 50 uM biotin. After 3 h of IPTG induction (0.55 mM), cells are harvested by centrifugation at 7300×g for 15 min and cell pellets stored frozen in −20 C. Extraction of ERα and ERβ are performed using 5 g of cells suspended in 50 mL of extraction buffer (50 mM Tris, pH 8.0, 100 mM KCl, 4 mM EDTA, 4 mM DDT and 0.1 mM PMSF). The cell suspension is run twice through a Microfluidizer M-110L (Microfluidics) and centrifuged at 15,000×g for 60 min. The supernatant is aliquoted and stored in −70 C.

Dilute ERα-LBD or ERβ-LBD extracts in assay buffer (18 mM K₂HPO₄, 2 mM KH2PO₄, 20 mM Na₁MoO₄, 1 mM EDTA, 1 mM TCEP) 1:676 and 1:517 for alpha and beta respectively. The diluted receptor concentrations should be 900 fmol/L. Preincubate the extracts with streptavidin coated polyvinyltoluene SPA beads (RPNQ0007, GE Healthcare) at a concentration of 0.43 mg/mL for 1 hr at room temperature.

Test compounds are evaluated over a range of concentrations from 157 μM to 37.5 pM. The test compound stock solutions should be made in 100% DMSO at 5× of the final concentration desired for testing in the assay. The amount of DMSO in the test wells of the 384 well plate will be 20%. Add 18 μl aliquots of test compounds to the assay plates followed by 35 μl of the preincubated receptor/SPA bead mix and finally add 35 μl of 3 nM ³H-E2. Cover the plates with a plastic sealer, centrifuge for 1 minute at 1000 rpm and equilibrate over night on a shaker at room temperature. The following morning, centrifuge the plates 5 minutes at 2000 rpm and measure on a plate scintillation counter e.g. a PerkinElmer Microbeta 1450 Trilux.

For compounds able to displace 3[H]-E2 from the receptor an IC₅₀-value (the concentration required to inhibit 50% of the binding of 3[H]-E2) is determined by a non-linear four parameter logistic model; b=((bmax−bmin)/(1+(I/IC₅₀)S))+bmin I is added concentration of binding inhibitor, IC₅₀ is the concentration of inhibitor at half maximal binding and S is a slope factor. The Microbeta-instrument generates the mean cpm (counts per minute) value/minute and corrects for individual variations between the detectors thus generating corrected cpm values.

The compounds of Examples 1-32 exhibit binding affinities to the estrogen receptor α-subtype in the range of IC₅₀ 1 to 10,000 nM and to the estrogen receptor β-subtype in the range of IC₅₀ 1 to 10,000 nM.

1. A compound of formula (I) or a pharmaceutically acceptable ester, amide, solvate or salt thereof, including a salt of such an ester or amide, and a solvate of such an ester, amide or salt, 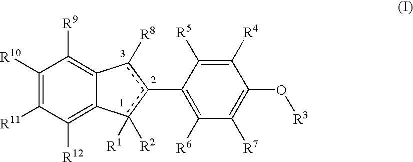
wherein either the bond between the C1 and C2 carbon atoms is a double bond or the bond between the C2 and C3 carbon atoms is a double bond, R² being absent when the bond between the C1 and C2 carbon atoms is a double bond;
R¹ and R² are independently selected from the group consisting of hydrogen, OR^{A}, C₁₋₆ alkyl, C₂₋₆ alkenyl, C₂₋₆ alkynyl, C₃₋₈ cycloalkyl, C₃₋₈ cycloalkyl C₁₋₆ alkyl, C₆₋₁₀ aryl, C₆₋₁₀ aryl C₁₋₆ alkyl, halogen, halo C₁₋₆ alkyl, dihalo C₁₋₆ alkyl and trihalo C₁₋₆ alkyl; or R¹ and R² taken together with the carbon atom to which they are attached form a double bond portion of C₂₋₆ alkenyl group;
R^{A} is selected from the group consisting of hydrogen, C₁₋₆ alkyl, C₂₋₆ alkenyl, C₂₋₆ alkynyl, C₃₋₈ cycloalkyl, C₃₋₈ cycloalkyl C₁₋₆ alkyl, C₆₋₁₀ aryl and C₆₋₁₀ aryl C₁₋₆ alkyl;
R³ is selected from the group consisting of hydrogen, C₁₋₆ alkyl, C₃₋₈ cycloalkyl and —C(O)C₁₋₄ alkyl;
R⁴, R⁵, R⁶ and R⁷ are the same or are different and each is selected from the group consisting of hydrogen, OR^{A}, halogen, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl;
R⁸ is selected from the group consisting of C₃₋₈ cycloalkyl, C₃₋₈ cycloalkyl C₁₋₆ alkyl, phenyl, benzyl and C₅₋₁₀ heterocyclyl wherein said phenyl, benzyl or C₅₋₁₀ heterocyclyl group can either be unsubstituted or substituted with 1-3 substituents and each substituent is selected from the group consisting of OR^{A}, halogen, cyano, nitro, C₁₋₆ alkyl, C₂₋₆ alkenyl, C₂₋₆ alkynyl, halo C₁₋₆ alkyl, dihalo C₁₋₆ alkyl, trihalo C₁₋₆ alkyl and C(O)C₁₋₆ alkyl;
R¹⁰ is OR^{A}; and
R⁹, R¹¹ and R¹² are the same or are different and each is selected from the group consisting of hydrogen, OR^{A}, halogen, cyano, nitro, C₁₋₆ alkyl, C₂₋₆ alkenyl, C₂₋₆ alkynyl, C(O)H, C(O)C₁₋₆ alkyl, halo C₁₋₆ alkyl, dihalo C₁₋₆ alkyl and trihalo C₁₋₆ alkyl;
with the proviso that the compound is not 4,5,6-trimethoxy-2-(4-methoxyphenyl)-1-methyl-3-phenyl-1H-indenol, 3-benzyl-2-(3,4-dimethoxyphenyl)-5,6-dimethoxyindene, or (R)-3-(3,5-dihydroxy-phenyl)-1-(4-hydroxy-benzyl)-2-(4-hydroxy-phenyl)-3H-indene-4,6-diol.

2. A compound as claimed in claim 1 wherein R³ is hydrogen.

3. A compound as claimed in claim 1 wherein R⁸ is selected from the group consisting of phenyl, benzyl and C₅₋₁₀ heterocyclyl wherein said phenyl, benzyl or C₅₋₁₀ heterocyclyl group can either be unsubstituted or substituted with 1-3 substituents and each substituent is selected from the group consisting of hydroxyl, halogen, cyano, methyl, ethyl, propyl, isopropyl and trifluoromethyl.

4. A compound as claimed in claim 3 wherein the C₅₋₁₀ heterocyclyl group is selected from thiophenyl, thiazolyl, furanyl, pyrazolyl, pyrrolyl, oxazolyl and imidazolyl.

5. A compound as claimed in claim 1
wherein either the bond between the C1 and C2 carbon atoms is a double bond or the bond between the C2 and C3 carbon atoms is a double bond, R² being absent when the bond between the C1 and C2 carbon atoms is a double bond;
R¹ and R² are independently selected from the group consisting of hydrogen, OR^{A}, C₁₋₆ alkyl, halogen, halo C₁₋₆ alkyl, dihalo C₁₋₆ alkyl and trihalo C₁₋₆ alkyl;
R^{A} is selected from the group consisting of hydrogen, C₁₋₄ alkyl and C₃₋₆ cycloalkyl;
R³ is selected from the group consisting of hydrogen, C₁₋₄ alkyl, C₃₋₆ cycloalkyl and —C(O)C₁₋₄ alkyl;
R⁴, R⁵, R⁶ and R⁷ are the same or are different and each is selected from the group consisting of hydrogen, OR^{A}, halogen, cyano, nitro, C₁₋₄ alkyl, halo C₁₋₄ alkyl, dihalo C₁₋₄ alkyl and trihalo C₁₋₄ alkyl;
R⁸ is selected from the group consisting of C₃₋₆ cycloalkyl, C₃₋₆ cycloalkyl C₁₋₄ alkyl, phenyl, benzyl and C₅₋₆ heterocyclyl wherein said phenyl, benzyl or C₅₋₆ heterocyclyl group can either be unsubstituted or substituted with 1-3 substituents and each substituent is selected from the group consisting of OR^{A}, halogen, cyano, nitro, C₁₋₄ alkyl, halo C₁₋₄ alkyl, dihalo C₁₋₄ alkyl, trihalo C₁₋₄ alkyl and C(O)C₁₋₆ alkyl;
R¹⁰ is OR^{A}; and
R⁹, R¹¹ and R¹² are the same or are different and each is selected from the group consisting of hydrogen, OR^{a}, halogen, cyano, nitro, C₁₋₄ alkyl, halo C₁₋₄ alkyl, dihalo C₁₋₄ alkyl and trihalo C₁₋₄ alkyl.

6. A compound as claimed in claim 1 which is selected from:
2-(4-Hydroxy-phenyl)-7-methyl-3-p-tolyl-1H-inden-5-ol;
3-(4-Fluoro-phenyl)-2-(4-hydroxy-phenyl)-7-methyl-1H-inden-5-ol
3-(4-Hydroxy-2-methyl-phenyl)-2-(4-hydroxy-phenyl)-7-methyl-1H-inden-5-ol;
2-(4-Hydroxy-phenyl)-3-phenyl-1H-inden-5-ol;
2-(4-Hydroxy-phenyl)-3-thiophen-2-yl-1H-inden-5-ol;
7-Ethyl-2-(4-hydroxy-phenyl)-3-phenyl-1H-inden-5-ol;
2-(4-Hydroxy-phenyl)-3-phenyl-7-propyl-1H-inden-5-ol;
2-(4-Hydroxy-phenyl)-7-methyl-3-phenyl-1H-inden-5-ol;
2-(4-Hydroxy-phenyl)-1-phenyl-3H-indene-4,6-diol;
6,7-Difluoro-2-(4-hydroxy-phenyl)-3-phenyl-1H-inden-5-ol;
2-(2-Fluoro-4-hydroxy-phenyl)-7-methyl-3-phenyl-1H-inden-5-ol;
2-(3-Fluoro-4-hydroxy-phenyl)-7-methyl-3-phenyl-1H-inden-5-ol;
2-(3-Chloro-4-hydroxy-phenyl)-7-methyl-3-phenyl-1H-inden-5-ol;
2-(3-Bromo-4-hydroxy-phenyl)-7-methyl-3-phenyl-1H-inden-5-ol;
2-(3,5-Dibromo-4-hydroxy-phenyl)-7-methyl-3-phenyl-3H-inden-5-ol;
2-(2,5-Dichloro-4-hydroxy-phenyl)-7-methyl-3-phenyl-3H-inden-5-ol;
2-(4-Hydroxy-phenyl)-7-methyl-3-thiophen-3-yl-1H-inden-5-ol;
2-(4-Hydroxy-phenyl)-7-methyl-3-thiophen-2-yl-1H-inden-5-ol;
7-Bromo-2-(4-hydroxy-phenyl)-3-phenyl-1H-inden-5-ol;
6-Hydroxy-2-(4-hydroxy-phenyl)-1-phenyl-3H-indene-4-carbonitrile;
6-Hydroxy-2-(4-hydroxy-phenyl)-1-phenyl-3H-indene-4-carbaldehyde;
1-Butyl-2-(4-hydroxy-phenyl)-3-phenyl-1H-inden-5-ol;
2-(4-Hydroxy-phenyl)-7-methyl-3-phenyl-1H-indene-1,5-diol;
2-(4-Hydroxy-phenyl)-7-methyl-1-methylene-3-phenyl-1H-inden-5-ol;
2-(4-Hydroxy-phenyl)-1-methyl-3-phenyl-3H-inden-5-ol;
2-(4-Hydroxy-phenyl)-1-isobutyl-3-phenyl-3H-inden-5-ol:
1-Butyl-2-(4-hydroxy-phenyl)-3-phenyl-3H-inden-5-ol;
1-Ethyl-2-(4-hydroxy-phenyl)-3-phenyl-3H-inden-5-ol;
2-(4-Hydroxy-phenyl)-3-phenyl-1-propyl-3H-inden-5-ol;
2-(4-Hydroxy-phenyl)-1-pentyl-3-phenyl-3H-inden-5-ol;
2-(4-hydroxy-phenyl)-3-(2-cyano-phenyl)-1H-inden-5-ol;
4-[6-Hydroxy-2-(4-hydroxy-phenyl)-4-methyl-3H-inden-1-yl]-thiophene-3-carbonitrile;
2-(4-Hydroxy-phenyl)-7-methyl-3-(3-methyl-thiophen-2-yl)-1H-inden-5-ol;
2-(2,3-Difluoro-4-hydroxy-phenyl)-7-methyl-3-phenyl-1H-inden-5-ol;
2-(2-Chloro-4-hydroxy-phenyl)-7-methyl-3-phenyl-1H-inden-5-ol;
2-(4-Hydroxy-3-methyl-phenyl)-7-methyl-3-phenyl-1H-inden-5-ol;
2-(4-Hydroxy-2-methyl-phenyl)-7-methyl-3-phenyl-1H-inden-5-ol;
3-[6-Hydroxy-2-(4-hydroxy-phenyl)-4-methyl-3H-inden-1-yl]-thiophene-2-carbonitrile;
2-(4-Hydroxy-phenyl)-7-methyl-3-thiazol-5-yl-1H-inden-5-ol;
3-(2-Ethyl-phenyl)-2-(4-hydroxy-phenyl)-7-methyl-1H-inden-5-ol;
2-(4-Hydroxy-phenyl)-3-(2-isopropyl-phenyl)-7-methyl-1H-inden-5-ol;
3-[2-(3-Fluoro-4-hydroxy-phenyl)-6-hydroxy-4-methyl-3H-inden-1-yl]-thiophene-2-carbonitrile;
3-[2-(3-Chloro-4-hydroxy-phenyl)-6-hydroxy-4-methyl-3H-inden-1-yl]-thiophene-2-carbonitrile;
3-[2-(2,3-Difluoro-4-hydroxy-phenyl)-6-hydroxy-4-methyl-3H-inden-1-yl]-thiophene-2-carbonitrile;
3-[6-Hydroxy-2-(4-hydroxy-phenyl)-4-trifluoromethyl-3H-inden-1-yl]-thiophene-2-carbonitrile;
3-[2-(3-Fluoro-4-hydroxy-phenyl)-6-hydroxy-4-trifluoromethyl-3H-inden-1-yl]-thiophene-2-carbonitrile;
3-[2-(3-Chloro-4-hydroxy-phenyl)-6-hydroxy-4-trifluoromethyl-3H-inden-1-yl]-thiophene-2-carbonitrile;
4-[2-(2,3-Difluoro-4-hydroxy-phenyl)-6-hydroxy-4-methyl-3H-inden-1-yl]-thiophene-3-carbonitrile;
4-[6-Hydroxy-2-(4-hydroxy-phenyl)-4-trifluoromethyl-3H-inden-1-yl]-thiophene-3-carbonitrile;
4-[2-(3-Fluoro-4-hydroxy-phenyl)-6-hydroxy-4-trifluoromethyl-3H-inden-1-yl]-thiophene-3-carbonitrile;
4-[2-(2,3-Difluoro-4-hydroxy-phenyl)-6-hydroxy-4-trifluoromethyl-3H-inden-1-yl]-thiophene-3-carbonitrile;
3-[2-(2,3-Difluoro-4-hydroxy-phenyl)-6-hydroxy-4-trifluoromethyl-3H-inden-1-yl]-thiophene-2-carbonitrile;
3-[2-(2,5-Difluoro-4-hydroxy-phenyl)-6-hydroxy-4-trifluoromethyl-3H-inden-1-yl]-thiophene-2-carbonitrile;
3-[2-(2,6-Difluoro-4-hydroxy-phenyl)-6-hydroxy-4-trifluoromethyl-3H-inden-1-yl]-thiophene-2-carbonitrile;
4-[2-(2,5-Difluoro-4-hydroxy-phenyl)-6-hydroxy-4-trifluoromethyl-3H-inden-1-yl]-thiophene-3-carbonitrile;
2-[6-Hydroxy-2-(4-hydroxy-phenyl)-4-methyl-3H-inden-1-yl]-furan-3-carbonitrile;
2-[5-Bromo-6-hydroxy-2-(4-hydroxy-phenyl)-4-methyl-3H-inden-1-yl]-furan-3-carbonitrile;
2-(4-Hydroxy-phenyl)-3-phenyl-7-trifluoromethyl-1H-inden-5-ol;
2-(2,6-Difluoro-4-hydroxy-phenyl)-7-methyl-3-phenyl-1H-inden-5-ol;
2-(2,5-Difluoro-4-hydroxy-phenyl)-7-methyl-3-phenyl-1H-inden-5-ol;
2-(4-Hydroxy-3-trifluoromethyl-phenyl)-7-methyl-3-phenyl-1H-inden-5-ol;
2-(4-Hydroxy-2,6-dimethyl-phenyl)-7-methyl-3-phenyl-1H-inden-5-ol;
2-(3,5-Dichloro-4-hydroxy-phenyl)-7-methyl-3-phenyl-1H-inden-5-ol;
2-(4-Hydroxy-phenyl)-7-methyl-3-thiazol-2-yl-1H-inden-5-ol;
1-{3-[6-Hydroxy-2-(4-hydroxy-phenyl)-4-methyl-3H-inden-1-yl]-furan-2-yl}-pentan-1-one;
4-[2-(3-Fluoro-4-hydroxy-phenyl)-6-hydroxy-4-methyl-3H-inden-1-yl]-thiophene-3-carbonitrile;
2-[2-(3-Fluoro-4-hydroxy-phenyl)-6-hydroxy-4-methyl-3H-inden-1-yl]-thiophene-3-carbonitrile;
4-[2-(2,5-Difluoro-4-hydroxy-phenyl)-6-hydroxy-4-methyl-3H-inden-1-yl]-thiophene-3-carbonitrile;
2-[2-(2,5-Difluoro-4-hydroxy-phenyl)-6-hydroxy-4-methyl-3H-inden-1-yl]-thiophene-3-carbonitrile;
4-Fluoro-2-(4-hydroxy-phenyl)-3-phenyl-1H-inden-5-ol;
4-Fluoro-2-(3-fluoro-4-hydroxy-phenyl)-3-phenyl-1H-inden-5-ol;
2-(2,3-Difluoro-4-hydroxy-phenyl)-4-fluoro-3-phenyl-1H-inden-5-ol;
4,6-Difluoro-2-(4-hydroxy-phenyl)-3-phenyl-1H-inden-5-ol;
4,6-Difluoro-2-(3-fluoro-4-hydroxy-phenyl)-3-phenyl-1H-inden-5-ol;
2-(2,3-Difluoro-4-hydroxy-phenyl)-4,6-difluoro-3-phenyl-1H-inden-5-ol;
3-[7-Fluoro-6-hydroxy-2-(4-hydroxy-phenyl)-3H-inden-1-yl]-thiophene-2-carbonitrile;
3-[7-Fluoro-2-(3-fluoro-4-hydroxy-phenyl)-6-hydroxy-3H-inden-1-yl]-thiophene-2-carbonitrile;
3-[2-(2,3-Difluoro-4-hydroxy-phenyl)-7-fluoro-6-hydroxy-3H-inden-1-yl]-thiophene-2-carbonitrile;
4-[2-(2,3-Difluoro-4-hydroxy-phenyl)-6-hydroxy-3H-inden-1-yl]-thiophene-3-carbonitrile;
4-[6-Hydroxy-2-(4-hydroxy-phenyl)-3H-inden-1-yl]-thiophene-3-carbonitrile;
2-(4-Hydroxy-phenyl)-3-(2-isobutyl-2H-pyrazol-3-yl)-7-methyl-1H-inden-5-ol;
3-(1-Ethyl-1H-pyrrol-2-yl)-2-(4-hydroxy-phenyl)-7-methyl-1H-inden-5-ol;
2-(4-Hydroxy-phenyl)-7-methyl-3-(1-methyl-1H-imidazol-2-yl)-1H-inden-5-ol;
2-(3-Fluoro-4-hydroxy-phenyl)-7-methyl-3-(2-methyl-2H-pyrazol-3-yl)-1H-inden-5-ol;
2-(2,3-Difluoro-4-hydroxy-phenyl)-7-methyl-3-(2-methyl-2H-pyrazol-3-yl)-1H-inden-5-ol;
2-(4-Hydroxy-phenyl)-7-methyl-3-(2-methyl-2H-pyrazol-3-yl)-1H-inden-5-ol;
3-(2-Ethyl-2H-pyrazol-3-yl)-2-(4-hydroxy-phenyl)-7-methyl-1H-inden-5-ol;
7-Difluoromethyl-2-(4-hydroxy-phenyl)-3-phenyl-1H-inden-5-ol;
7-Fluoromethyl-2-(4-hydroxy-phenyl)-3-phenyl-1H-inden-5-ol;
7-Bromomethyl-2-(4-hydroxy-phenyl)-3-phenyl-1H-inden-5-ol;
2-(4-Hydroxy-phenyl)-3-phenyl-7-vinyl-1H-inden-5-ol;
4-[2-(3-Fluoro-4-methoxy-phenyl)-6-methoxy-4-methyl-3H-inden-1-yl]-thiophene-3-carbonitrile;
4-[4-Fluoro-6-hydroxy-2-(4-hydroxy-phenyl)-3H-inden-1-yl]-thiophene-3-carbonitrile;
4-[2-(2,3-Difluoro-4-hydroxy-phenyl)-4-fluoro-6-hydroxy-3H-inden-1-yl]-thiophene-3-carbonitrile;
4-[5,7-Difluoro-2-(3-fluoro-4-hydroxy-phenyl)-6-hydroxy-3H-inden-1-yl]-thiophene-3-carbonitrile;
2-[4-Fluoro-6-hydroxy-2-(4-hydroxy-phenyl)-3H-inden-1-yl]-furan-3-carbonitrile;
3-[6-Hydroxy-2-(4-hydroxy-phenyl)-4-methyl-3H-inden-1-yl]-furan-2-carbonitrile;
3-[2-(3-Fluoro-4-hydroxy-phenyl)-6-hydroxy-4-methyl-3H-inden-1-yl]-furan-2-carbonitrile;
3-[2-(2,3-Difluoro-4-hydroxy-phenyl)-6-hydroxy-4-methyl-3H-inden-1-yl]-furan-2-carbonitrile;
5-[6-Hydroxy-2-(4-hydroxy-phenyl)-4-trifluoromethyl-3H-inden-1-yl]-furan-2-carbonitrile;
5-[2-(3-Fluoro-4-hydroxy-phenyl)-6-hydroxy-4-trifluoromethyl-3H-inden-1-yl]-furan-2-carbonitrile;
2-[6-Hydroxy-2-(4-hydroxy-phenyl)-4-trifluoromethyl-3H-inden-1-yl]-furan-3-carbonitrile;
2-[2-(3-Fluoro-4-hydroxy-phenyl)-6-hydroxy-4-trifluoromethyl-3H-inden-1-yl]-furan-3-carbonitrile;
3-[6-Hydroxy-2-(4-hydroxy-phenyl)-4-trifluoromethyl-3H-inden-1-yl]-furan-2-carbonitrile;
3-[2-(3-Fluoro-4-hydroxy-phenyl)-6-hydroxy-4-trifluoromethyl-3H-inden-1-yl]-furan-2-carbonitrile;
3-[6-Hydroxy-2-(4-hydroxy-phenyl)-4-methyl-3H-inden-1-yl]-furan-2-carbaldehyde;
4-[2-(2,4-Dihydroxy-phenyl)-6-hydroxy-4-methyl-3H-inden-1-yl]-thiophene-3-carbonitrile;
7-Chloro-2-(4-hydroxy-phenyl)-3-phenyl-1H-inden-5-ol;
4-[4-Chloro-2-(3-fluoro-4-hydroxy-phenyl)-6-hydroxy-3H-inden-1-yl]-thiophene-3-carbonitrile;
2-[4-Chloro-6-hydroxy-2-(4-hydroxy-phenyl)-3H-inden-1-yl]-furan-3-carbonitrile;
2-[4-Chloro-2-(3-fluoro-4-hydroxy-phenyl)-6-hydroxy-3H-inden-1-yl]-furan-3-carbonitrile;
2-[2-(2,3-Difluoro-4-hydroxy-phenyl)-6-hydroxy-4-methyl-3H-inden-1-yl]-furan-3-carbonitrile;
2-[2-(2,3-Difluoro-4-hydroxy-phenyl)-7-fluoro-6-hydroxy-3H-inden-1-yl]-furan-3-carbonitrile;
2-[2-(3-Fluoro-4-hydroxy-phenyl)-7-fluoro-6-hydroxy-3H-inden-1-yl]-furan-3-carbonitrile;
2-[2-(3-Chloro-5-fluoro-4-hydroxy-phenyl)-7-fluoro-6-hydroxy-3H-inden-1-yl]-furan-3-carbonitrile;
2-[2-(2,3-Difluoro-4-hydroxy-phenyl)-4-fluoro-6-hydroxy-3H-inden-1-yl]-furan-3-carbonitrile;
2-[6-Hydroxy-2-(4-hydroxy-phenyl)-4-methyl-3H-inden-1-yl]-1-methyl-1H-imidazole-4-carbonitrile;
2-[4-Chloro-2-(2,3-difluoro-4-hydroxy-phenyl)-6-hydroxy-3H-inden-1-yl]-furan-3-carbonitrile;
2-[2-(2,3-Difluoro-4-hydroxy-phenyl)-6-hydroxy-4-trifluoromethyl-3H-inden-1-yl]-furan-3-carbonitrile;
2-[6-Hydroxy-2-(4-hydroxy-phenyl)-3,4-dimethyl-3H-inden-1-yl]-furan-3-carbonitrile;
2-[2-(3-Chloro-5-fluoro-4-hydroxy-phenyl)-6-hydroxy-4-methyl-3H-inden-1-yl]-furan-3-carbonitrile;
2-[6-Methoxy-2-(4-methoxy-phenyl)-4-methyl-3H-inden-1-yl]-furan-3-carbonitrile;
2-[7-Fluoro-6-hydroxy-2-(4-hydroxy-phenyl)-3H-inden-1-yl]-furan-3-carbonitrile;
2-[4-Chloro-2-(3-chloro-5-fluoro-4-hydroxy-phenyl)-6-hydroxy-3H-inden-1-yl]-furan-3-carbonitrile;
2-[4-Fluoro-2-(3-fluoro-4-hydroxy-phenyl)-6-hydroxy-3H-inden-1-yl]-furan-3-carbonitrile;
2-[2-(3-Chloro-5-fluoro-4-hydroxy-phenyl)-4-fluoro-6-hydroxy-3H-inden-1-yl]-furan-3-carbonitrile;
2-[2-(3-Chloro-5-fluoro-4-hydroxy-phenyl)-6-hydroxy-4-trifluoromethyl-3H-inden-1-yl]-furan-3-carbonitrile;
or a pharmaceutically acceptable ester, amide, solvate or salt thereof, including a salt of such an ester or amide, and a solvate of such an ester, amide or salt.

7. A method for the treatment or prophylaxis of a condition associated with a disease or disorder associated with estrogen receptor activity in a mammal, which comprises administering to the mammal a therapeutically effective amount of a compound of formula (I) or a pharmaceutically acceptable ester, amide, solvate or salt thereof, including a salt of such an ester or amide, and a solvate of such an ester, amide or salt, 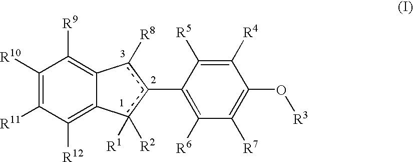
wherein either the bond between the C1 and C2 carbon atoms is a double bond or the bond between the C2 and C3 carbon atoms is a double bond, R² being absent when the bond between the C1 and C2 carbon atoms is a double bond;
R¹ and R² are independently selected from the group consisting of hydrogen, OR^{A}, C₁₋₆ alkyl, C₂₋₆ alkenyl, C₂₋₆ alkynyl, C₃₋₈ cycloalkyl, C₃₋₈ cycloalkyl C₁₋₆ alkyl, C₆₋₁₀ aryl, C₆₋₁₀ aryl C₁₋₆ alkyl, halogen, halo C₁₋₆ alkyl, dihalo C₁₋₆ alkyl and trihalo C₁₋₆ alkyl; or R¹ and R² taken together with the carbon atom to which they are attached form a double bond portion of C₂₋₆ alkenyl group;
R^{A} is selected from the group consisting of hydrogen, C₁₋₆ alkyl, C₂₋₆ alkenyl, C₂₋₆ alkynyl, C₃₋₈ cycloalkyl, C₃₋₈ cycloalkyl C₁₋₆ alkyl, C₆₋₁₀ aryl and C₆₋₁₀ aryl C₁₋₆ alkyl;
R³ is selected from the group consisting of hydrogen, C₁₋₆ alkyl, C₃₋₈ cycloalkyl and —C(O)C₁₋₄ alkyl;
R⁴, R⁵, R⁶ and R⁷ are the same or are different and each is selected from the group consisting of hydrogen, OR^{A}, halogen, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl;
R⁸ is selected from the group consisting of C₃₋₈ cycloalkyl, C₃₋₈ cycloalkyl C₁₋₆ alkyl, phenyl, benzyl and C₅₋₁₀ heterocyclyl wherein said phenyl, benzyl or C₅₋₁₀ heterocyclyl group can either be unsubstituted or substituted with 1-3 substituents and each substituent is selected from the group consisting of OR^{A}, halogen, cyano, nitro, C₁₋₆ alkyl, C₂₋₆ alkenyl, C₂₋₆ alkynyl, halo C₁₋₆ alkyl, dihalo C₁₋₆ alkyl, trihalo C₁₋₆ alkyl and C(O)C₁₋₆ alkyl;
R¹⁰ is OR^{A}; and
R⁹, R¹¹ and R¹² are the same or are different and each is selected from the group consisting of hydrogen, OR^{A}, halogen, cyano, nitro, C₁₋₆ alkyl, C₂₋₆ alkenyl, C₂₋₆ alkynyl, C(O)H, C(O)C₁₋₆ alkyl, halo C₁₋₆ alkyl, dihalo C₁₋₆ alkyl and trihalo C₁₋₆ alkyl.

8. A method as claimed in claim 7 wherein R³ is hydrogen.

9. A method as claimed in claim 7 wherein R⁸ is selected from the group consisting of phenyl, benzyl and C₅₋₁₀ heterocyclyl wherein said phenyl, benzyl or C₅₋₁₀ heterocyclyl group can either be unsubstituted or substituted with 1-3 substituents and each substituent is selected from the group consisting of hydroxyl, halogen, cyano, methyl, ethyl, propyl, isopropyl and trifluoromethyl.

10. A method as claimed in claim 9 wherein the C₅₋₁₀ heterocyclyl group is selected from thiophenyl, thiazolyl, furanyl, pyrazolyl, pyrrolyl, oxazolyl and imidazolyl.

11. A method as claimed in claim 7 wherein either the bond between the C1 and C2 carbon atoms is a double bond or the bond between the C2 and C3 carbon atoms is a double bond, R² being absent when the bond between the C1 and C2 carbon atoms is a double bond;
R¹ and R² are independently selected from the group consisting of hydrogen, OR^{A}, C₁₋₆ alkyl, halogen, halo C₁₋₆ alkyl, dihalo C₁₋₆ alkyl and trihalo C₁₋₆ alkyl;
R^{A} is selected from the group consisting of hydrogen, C₁₋₄ alkyl and C₃₋₆ cycloalkyl;
R³ is selected from the group consisting of hydrogen, C₁₋₄ alkyl, C₃₋₆ cycloalkyl and —C(O)C₁₋₄ alkyl;
R⁴, R⁵, R⁶ and R⁷ are the same or are different and each is selected from the group consisting of hydrogen, OR^{A}, halogen, cyano, nitro, C₁₋₄ alkyl, halo C₁₋₄ alkyl, dihalo C₁₋₄ alkyl and trihalo C₁₋₄ alkyl;
R⁸ is selected from the group consisting of C₃₋₆ cycloalkyl, C₃₋₆ cycloalkyl C₁₋₄ alkyl, phenyl, benzyl and C₅₋₆ heterocyclyl wherein said phenyl, benzyl or C₅₋₆ heterocyclyl group can either be unsubstituted or substituted with 1-3 substituents and each substituent is selected from the group consisting of OR^{A}, halogen, cyano, nitro, C₁₋₄ alkyl, halo C₁₋₄ alkyl, dihalo C₁₋₄ alkyl, trihalo C₁₋₄ alkyl and C(O)C₁₋₆ alkyl;
R¹⁰ is OR^{A}; and
R⁹, R¹¹ and R¹² are the same or are different and each is selected from the group consisting of hydrogen, OR^{A}, halogen, cyano, nitro, C₁₋₄ alkyl, halo C₁₋₄ alkyl, dihalo C₁₋₄ alkyl and trihalo C₁₋₄ alkyl.

12. A method as claimed in claim 7 which comprises administering to the mammal a therapeutically effective amount of a compound of formula (I) which is selected from
2-(4-Hydroxy-phenyl)-7-methyl-3-p-tolyl-1H-inden-5-ol;
3-(4-Fluoro-phenyl)-2-(4-hydroxy-phenyl)-7-methyl-1H-inden-5-ol;
3-(4-Hydroxy-2-methyl-phenyl)-2-(4-hydroxy-phenyl)-7-methyl-1H-inden-5-ol;
2-(4-Hydroxy-phenyl)-3-phenyl-1H-inden-5-ol;
2-(4-Hydroxy-phenyl)-3-thiophen-2-yl-1H-inden-5-ol;
7-Ethyl-2-(4-hydroxy-phenyl)-3-phenyl-1H-inden-5-ol;
2-(4-Hydroxy-phenyl)-3-phenyl-7-propyl-1H-inden-5-ol;
2-(4-Hydroxy-phenyl)-7-methyl-3-phenyl-1H-inden-5-ol;
2-(4-Hydroxy-phenyl)-1-phenyl-3H-indene-4,6-diol;
6,7-Difluoro-2-(4-hydroxy-phenyl)-3-phenyl-1H-inden-5-ol;
2-(2-Fluoro-4-hydroxy-phenyl)-7-methyl-3-phenyl-1H-inden-5-ol;
2-(3-Fluoro-4-hydroxy-phenyl)-7-methyl-3-phenyl-1H-inden-5-ol;
2-(3-Chloro-4-hydroxy-phenyl)-7-methyl-3-phenyl-1H-inden-5-ol;
2-(3-Bromo-4-hydroxy-phenyl)-7-methyl-3-phenyl-1H-inden-5-ol;
2-(3,5-Dibromo-4-hydroxy-phenyl)-7-methyl-3-phenyl-3H-inden-5-ol;
2-(2,5-Dichloro-4-hydroxy-phenyl)-7-methyl-3-phenyl-3H-inden-5-ol;
2-(4-Hydroxy-phenyl)-7-methyl-3-thiophen-3-yl-1H-inden-5-ol;
2-(4-Hydroxy-phenyl)-7-methyl-3-thiophen-2-yl-1H-inden-5-ol;
7-Bromo-2-(4-hydroxy-phenyl)-3-phenyl-1H-inden-5-ol;
6-Hydroxy-2-(4-hydroxy-phenyl)-1-phenyl-3H-indene-4-carbonitrile;
6-Hydroxy-2-(4-hydroxy-phenyl)-1-phenyl-3H-indene-4-carbaldehyde;
1-Butyl-2-(4-hydroxy-phenyl)-3-phenyl-1H-inden-5-ol;
2-(4-Hydroxy-phenyl)-7-methyl-3-phenyl-1H-indene-1,5-diol;
2-(4-Hydroxy-phenyl)-7-methyl-1-methylene-3-phenyl-1H-inden-5-ol;
2-(4-Hydroxy-phenyl)-1-methyl-3-phenyl-3H-inden-5-ol;
2-(4-Hydroxy-phenyl)-1-isobutyl-3-phenyl-3H-inden-5-ol:
1-Butyl-2-(4-hydroxy-phenyl)-3-phenyl-3H-inden-5-ol;
1-Ethyl-2-(4-hydroxy-phenyl)-3-phenyl-3H-inden-5-ol;
2-(4-Hydroxy-phenyl)-3-phenyl-1-propyl-3H-inden-5-ol;
2-(4-Hydroxy-phenyl)-1-pentyl-3-phenyl-3H-inden-5-ol;
2-(4-hydroxy-phenyl)-3-(2-cyano-phenyl)-1H-inden-5-ol;
4-[6-Hydroxy-2-(4-hydroxy-phenyl)-4-methyl-3H-inden-1-yl]-thiophene-3-carbonitrile;
2-(4-Hydroxy-phenyl)-7-methyl-3-(3-methyl-thiophen-2-yl)-1H-inden-5-ol;
2-(2,3-Difluoro-4-hydroxy-phenyl)-7-methyl-3-phenyl-1H-inden-5-ol;
2-(2-Chloro-4-hydroxy-phenyl)-7-methyl-3-phenyl-1H-inden-5-ol;
2-(4-Hydroxy-3-methyl-phenyl)-7-methyl-3-phenyl-1H-inden-5-ol;
2-(4-Hydroxy-2-methyl-phenyl)-7-methyl-3-phenyl-1H-inden-5-ol;
3-[6-Hydroxy-2-(4-hydroxy-phenyl)-4-methyl-3H-inden-1-yl]-thiophene-2-carbonitrile;
2-(4-Hydroxy-phenyl)-7-methyl-3-thiazol-5-yl-1H-inden-5-ol;
3-(2-Ethyl-phenyl)-2-(4-hydroxy-phenyl)-7-methyl-1H-inden-5-ol;
2-(4-Hydroxy-phenyl)-3-(2-isopropyl-phenyl)-7-methyl-1H-inden-5-ol;
3-[2-(3-Fluoro-4-hydroxy-phenyl)-6-hydroxy-4-methyl-3H-inden-1-yl]-thiophene-2-carbonitrile;
3-[2-(3-Chloro-4-hydroxy-phenyl)-6-hydroxy-4-methyl-3H-inden-1-yl]-thiophene-2-carbonitrile;
3-[2-(2,3-Difluoro-4-hydroxy-phenyl)-6-hydroxy-4-methyl-3H-inden-1-yl]-thiophene-2-carbonitrile;
3-[6-Hydroxy-2-(4-hydroxy-phenyl)-4-trifluoromethyl-3H-inden-1-yl]-thiophene-2-carbonitrile;
3-[2-(3-Fluoro-4-hydroxy-phenyl)-6-hydroxy-4-trifluoromethyl-3H-inden-1-yl]-thiophene-2-carbonitrile;
3-[2-(3-Chloro-4-hydroxy-phenyl)-6-hydroxy-4-trifluoromethyl-3H-inden-1-yl]-thiophene-2-carbonitrile;
4-[2-(2,3-Difluoro-4-hydroxy-phenyl)-6-hydroxy-4-methyl-3H-inden-1-yl]-thiophene-3-carbonitrile;
4-[6-Hydroxy-2-(4-hydroxy-phenyl)-4-trifluoromethyl-3H-inden-1-yl]-thiophene-3-carbonitrile;
4-[2-(3-Fluoro-4-hydroxy-phenyl)-6-hydroxy-4-trifluoromethyl-3H-inden-1-yl]-thiophene-3-carbonitrile;
4-[2-(2,3-Difluoro-4-hydroxy-phenyl)-6-hydroxy-4-trifluoromethyl-3H-inden-1-yl]-thiophene-3-carbonitrile;
3-[2-(2,3-Difluoro-4-hydroxy-phenyl)-6-hydroxy-4-trifluoromethyl-3H-inden-1-yl]-thiophene-2-carbonitrile;
3-[2-(2,5-Difluoro-4-hydroxy-phenyl)-6-hydroxy-4-trifluoromethyl-3H-inden-1-yl]-thiophene-2-carbonitrile;
3-[2-(2,6-Difluoro-4-hydroxy-phenyl)-6-hydroxy-4-trifluoromethyl-3H-inden-1-yl]-thiophene-2-carbonitrile;
4-[2-(2,5-Difluoro-4-hydroxy-phenyl)-6-hydroxy-4-trifluoromethyl-3H-inden-1-yl]-thiophene-3-carbonitrile;
2-[6-Hydroxy-2-(4-hydroxy-phenyl)-4-methyl-3H-inden-1-yl]-furan-3-carbonitrile;
2-[5-Bromo-6-hydroxy-2-(4-hydroxy-phenyl)-4-methyl-3H-inden-1-yl]-furan-3-carbonitrile;
2-(4-Hydroxy-phenyl)-3-phenyl-7-trifluoromethyl-1H-inden-5-ol;
2-(2,6-Difluoro-4-hydroxy-phenyl)-7-methyl-3-phenyl-1H-inden-5-ol;
2-(2,5-Difluoro-4-hydroxy-phenyl)-7-methyl-3-phenyl-1H-inden-5-ol;
2-(4-Hydroxy-3-trifluoromethyl-phenyl)-7-methyl-3-phenyl-1H-inden-5-ol;
2-(4-Hydroxy-2,6-dimethyl-phenyl)-7-methyl-3-phenyl-1H-inden-5-ol;
2-(3,5-Dichloro-4-hydroxy-phenyl)-7-methyl-3-phenyl-1H-inden-5-ol;
2-(4-Hydroxy-phenyl)-7-methyl-3-thiazol-2-yl-1H-inden-5-ol;
1-{3-[6-Hydroxy-2-(4-hydroxy-phenyl)-4-methyl-3H-inden-1-yl]-furan-2-yl}-pentan-1-one;
4-[2-(3-Fluoro-4-hydroxy-phenyl)-6-hydroxy-4-methyl-3H-inden-1-yl]-thiophene-3-carbonitrile;
2-[2-(3-Fluoro-4-hydroxy-phenyl)-6-hydroxy-4-methyl-3H-inden-1-yl]-thiophene-3-carbonitrile;
4-[2-(2,5-Difluoro-4-hydroxy-phenyl)-6-hydroxy-4-methyl-3H-inden-1-yl]-thiophene-3-carbonitrile;
2-[2-(2,5-Difluoro-4-hydroxy-phenyl)-6-hydroxy-4-methyl-3H-inden-1-yl]-thiophene-3-carbonitrile;
4-Fluoro-2-(4-hydroxy-phenyl)-3-phenyl-1H-inden-5-ol;
4-Fluoro-2-(3-fluoro-4-hydroxy-phenyl)-3-phenyl-1H-inden-5-ol;
2-(2,3-Difluoro-4-hydroxy-phenyl)-4-fluoro-3-phenyl-1H-inden-5-ol;
4,6-Difluoro-2-(4-hydroxy-phenyl)-3-phenyl-1H-inden-5-ol;
4,6-Difluoro-2-(3-fluoro-4-hydroxy-phenyl)-3-phenyl-1H-inden-5-ol;
2-(2,3-Difluoro-4-hydroxy-phenyl)-4,6-difluoro-3-phenyl-1H-inden-5-ol;
3-[7-Fluoro-6-hydroxy-2-(4-hydroxy-phenyl)-3H-inden-1-yl]-thiophene-2-carbonitrile;
3-[7-Fluoro-2-(3-fluoro-4-hydroxy-phenyl)-6-hydroxy-3H-inden-1-yl]-thiophene-2-carbonitrile;
3-[2-(2,3-Difluoro-4-hydroxy-phenyl)-7-fluoro-6-hydroxy-3H-inden-1-yl]-thiophene-2-carbonitrile;
4-[2-(2,3-Difluoro-4-hydroxy-phenyl)-6-hydroxy-3H-inden-1-yl]-thiophene-3-carbonitrile;
4-[6-Hydroxy-2-(4-hydroxy-phenyl)-3H-inden-1-yl]-thiophene-3-carbonitrile;
2-(4-Hydroxy-phenyl)-3-(2-isobutyl-2H-pyrazol-3-yl)-7-methyl-1H-inden-5-ol;
3-(1-Ethyl-1H-pyrrol-2-yl)-2-(4-hydroxy-phenyl)-7-methyl-1H-inden-5-ol;
2-(4-Hydroxy-phenyl)-7-methyl-3-(1-methyl-1H-imidazol-2-yl)-1H-inden-5-ol;
2-(3-Fluoro-4-hydroxy-phenyl)-7-methyl-3-(2-methyl-2H-pyrazol-3-yl)-1H-inden-5-ol;
2-(2,3-Difluoro-4-hydroxy-phenyl)-7-methyl-3-(2-methyl-2H-pyrazol-3-yl)-1H-inden-5-ol;
2-(4-Hydroxy-phenyl)-7-methyl-3-(2-methyl-2H-pyrazol-3-yl)-1H-inden-5-ol;
3-(2-Ethyl-2H-pyrazol-3-yl)-2-(4-hydroxy-phenyl)-7-methyl-1H-inden-5-ol;
7-Difluoromethyl-2-(4-hydroxy-phenyl)-3-phenyl-1H-inden-5-ol;
7-Fluoromethyl-2-(4-hydroxy-phenyl)-3-phenyl-1H-inden-5-ol;
7-Bromomethyl-2-(4-hydroxy-phenyl)-3-phenyl-1H-inden-5-ol;
2-(4-Hydroxy-phenyl)-3-phenyl-7-vinyl-1H-inden-5-ol;
4-[2-(3-Fluoro-4-methoxy-phenyl)-6-methoxy-4-methyl-3H-inden-1-yl]-thiophene-3-carbonitrile;
4-[4-Fluoro-6-hydroxy-2-(4-hydroxy-phenyl)-3H-inden-1-yl]-thiophene-3-carbonitrile;
4-[2-(2,3-Difluoro-4-hydroxy-phenyl)-4-fluoro-6-hydroxy-3H-inden-1-yl]-thiophene-3-carbonitrile;
4-[5,7-Difluoro-2-(3-fluoro-4-hydroxy-phenyl)-6-hydroxy-3H-inden-1-yl]-thiophene-3-carbonitrile;
2-[4-Fluoro-6-hydroxy-2-(4-hydroxy-phenyl)-3H-inden-1-yl]-furan-3-carbonitrile;
3-[6-Hydroxy-2-(4-hydroxy-phenyl)-4-methyl-3H-inden-1-yl]-furan-2-carbonitrile;
3-[2-(3-Fluoro-4-hydroxy-phenyl)-6-hydroxy-4-methyl-3H-inden-1-yl]-furan-2-carbonitrile;
3-[2-(2,3-Difluoro-4-hydroxy-phenyl)-6-hydroxy-4-methyl-3H-inden-1-yl]-furan-2-carbonitrile;
5-[6-Hydroxy-2-(4-hydroxy-phenyl)-4-trifluoromethyl-3H-inden-1-yl]-furan-2-carbonitrile;
5-[2-(3-Fluoro-4-hydroxy-phenyl)-6-hydroxy-4-trifluoromethyl-3H-inden-1-yl]-furan-2-carbonitrile;
2-[6-Hydroxy-2-(4-hydroxy-phenyl)-4-trifluoromethyl-3H-inden-1-yl]-furan-3-carbonitrile;
2-[2-(3-Fluoro-4-hydroxy-phenyl)-6-hydroxy-4-trifluoromethyl-3H-inden-1-yl]-furan-3-carbonitrile;
3-[6-Hydroxy-2-(4-hydroxy-phenyl)-4-trifluoromethyl-3H-inden-1-yl]-furan-2-carbonitrile;
3-[2-(3-Fluoro-4-hydroxy-phenyl)-6-hydroxy-4-trifluoromethyl-3H-inden-1-yl]-furan-2-carbonitrile;
3-[6-Hydroxy-2-(4-hydroxy-phenyl)-4-methyl-3H-inden-1-yl]-furan-2-carbaldehyde;
4-[2-(2,4-Dihydroxy-phenyl)-6-hydroxy-4-methyl-3H-inden-1-yl]-thiophene-3-carbonitrile;
7-Chloro-2-(4-hydroxy-phenyl)-3-phenyl-1H-inden-5-ol;
4-[4-Chloro-2-(3-fluoro-4-hydroxy-phenyl)-6-hydroxy-3H-inden-1-yl]-thiophene-3-carbonitrile;
2-[4-Chloro-6-hydroxy-2-(4-hydroxy-phenyl)-3H-inden-1-yl]-furan-3-carbonitrile;
2-[4-Chloro-2-(3-fluoro-4-hydroxy-phenyl)-6-hydroxy-3H-inden-1-yl]-furan-3-carbonitrile;
2-[2-(2,3-Difluoro-4-hydroxy-phenyl)-6-hydroxy-4-methyl-3H-inden-1-yl]-furan-3-carbonitrile;
2-[2-(2,3-Difluoro-4-hydroxy-phenyl)-7-fluoro-6-hydroxy-3H-inden-1-yl]-furan-3-carbonitrile;
2-[2-(3-Fluoro-4-hydroxy-phenyl)-7-fluoro-6-hydroxy-3H-inden-1-yl]-furan-3-carbonitrile;
2-[2-(3-Chloro-5-fluoro-4-hydroxy-phenyl)-7-fluoro-6-hydroxy-3H-inden-1-yl]-furan-3-carbonitrile;
2-[2-(2,3-Difluoro-4-hydroxy-phenyl)-4-fluoro-6-hydroxy-3H-inden-1-yl]-furan-3-carbonitrile;
2-[6-Hydroxy-2-(4-hydroxy-phenyl)-4-methyl-3H-inden-1-yl]-1-methyl-1H-imidazole-4-carbonitrile;
2-[4-Chloro-2-(2,3-difluoro-4-hydroxy-phenyl)-6-hydroxy-3H-inden-1-yl]-furan-3-carbonitrile;
2-[2-(2,3-Difluoro-4-hydroxy-phenyl)-6-hydroxy-4-trifluoromethyl-3H-inden-1-yl]-furan-3-carbonitrile;
2-[6-Hydroxy-2-(4-hydroxy-phenyl)-3,4-dimethyl-3H-inden-1-yl]-furan-3-carbonitrile;
2-[2-(3-Chloro-5-fluoro-4-hydroxy-phenyl)-6-hydroxy-4-methyl-3H-inden-1-yl]-furan-3-carbonitrile;
2-[6-Methoxy-2-(4-methoxy-phenyl)-4-methyl-3H-inden-1-yl]-furan-3-carbonitrile;
2-[7-Fluoro-6-hydroxy-2-(4-hydroxy-phenyl)-3H-inden-1-yl]-furan-3-carbonitrile;
2-[4-Chloro-2-(3-chloro-5-fluoro-4-hydroxy-phenyl)-6-hydroxy-3H-inden-1-yl]-furan-3-carbonitrile;
2-[4-Fluoro-2-(3-fluoro-4-hydroxy-phenyl)-6-hydroxy-3H-inden-1-yl]-furan-3-carbonitrile;
2-[2-(3-Chloro-5-fluoro-4-hydroxy-phenyl)-4-fluoro-6-hydroxy-3H-inden-1-yl]-furan-3-carbonitrile;
2-[2-(3-Chloro-5-fluoro-4-hydroxy-phenyl)-6-hydroxy-4-trifluoromethyl-3H-inden-1-yl]-furan-3-carbonitrile;
or a pharmaceutically acceptable ester, amide, solvate or salt thereof, including a salt of such an ester or amide, and a solvate of such an ester, amide or salt.

13-15. (canceled)

16. A pharmaceutical composition comprising a compound of formula (I) or a pharmaceutically acceptable ester, amide, solvate or salt thereof, including a salt of such an ester or amide, and including a solvate of such an ester, amide or salt, 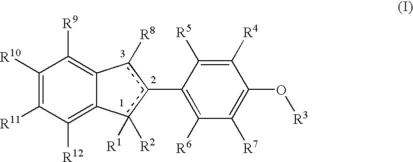
wherein either the bond between the C1 and C2 carbon atoms is a double bond or the bond between the C2 and C3 carbon atoms is a double bond, R² being absent when the bond between the C1 and C2 carbon atoms is a double bond;
R¹ and R² are independently selected from the group consisting of hydrogen, OR^{A}, C₁₋₆ alkyl, C₂₋₆ alkenyl, C₂₋₆ alkynyl, C₃₋₈ cycloalkyl, C₃₋₈ cycloalkyl C₁₋₆ alkyl, C₆₋₁₀ aryl, C₆₋₁₀ aryl C₁₋₆ alkyl, halogen, halo C₁₋₆ alkyl, dihalo C₁₋₆alkyl and trihalo C₁₋₆ alkyl; or R¹ and R² taken together with the carbon atom to which they are attached form a double bond portion of C₂₋₆ alkenyl group;
R^{A} is selected from the group consisting of hydrogen, C₁₋₆ alkyl, C₂₋₆ alkenyl, C₂₋₆ alkynyl, C₃₋₈ cycloalkyl, C₃₋₈ cycloalkyl C₁₋₆ alkyl, C₆₋₁₀ aryl and C₆₋₁₀ aryl C₁₋₆ alkyl;
R³ is selected from the group consisting of hydrogen, C₁₋₆ alkyl, C₃₋₈ cycloalkyl and —C(O)C₁₋₄ alkyl;
R⁴, R⁵, R⁶ and R⁷ are the same or are different and each is selected from the group consisting of hydrogen, OR^{A}, halogen, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl;
R⁸ is selected from the group consisting of C₃₋₈ cycloalkyl, C₃₋₈ cycloalkyl C₁₋₆ alkyl, phenyl, benzyl and C₅₋₁₀ heterocyclyl wherein said phenyl, benzyl or C₅₋₁₀ heterocyclyl group can either be unsubstituted or substituted with 1-3 substituents and each substituent is selected from the group consisting of OR^{A}, halogen, cyano, nitro, C₁₋₆ alkyl, C₂₋₆ alkenyl, C₂₋₆ alkynyl, halo C₁₋₆ alkyl, dihalo C₁₋₆ alkyl, trihalo C₁₋₆ alkyl and C(O)C₁₋₆ alkyl;
R¹⁰ is OR^{A}; and
R⁹, R¹¹ and R¹² are the same or are different and each is selected from the group consisting of hydrogen, OR^{A}, halogen, cyano, nitro, C₁₋₆ alkyl, C₂₋₆ alkenyl, C₂₋₆ alkynyl, C(O)H, C(O)C₁₋₆ alkyl, halo C₁₋₆ alkyl, dihalo C₁₋₆ alkyl and trihalo C₁₋₆ alkyl,
and a pharmaceutically acceptable carrier.

17. A pharmaceutical composition as claimed in claim 16 further comprising an additional therapeutic agent selected from: an organic bisphosphonate; a cathepsin K inhibitor; an estrogen; an estrogen receptor modulator; an androgen receptor modulator; an inhibitor of osteoclast proton ATPase; an inhibitor of HMG-CoA reductase; an integrin receptor antagonist; anosteoblast anabolic agent; calcitonin; Vitamin D; a synthetic Vitamin D analogue; an anti-depressant; an anxiolytic; or an anti-psychotic; or a pharmaceutically acceptable salt thereof or a mixture thereof.

18-19. (canceled)

20. A method as claimed in claim 7, wherein the condition associated with a disease or disorder associated with estrogen receptor activity is selected from bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression, autoimmune disease, inflammation, IBD, IBS, sexual dysfunction, hypertension, retinal degeneration and lung, colon, breast, uterus, and prostate cancer.

21. A compound as claimed in claim 2 wherein R⁸ is selected from the group consisting of phenyl, benzyl and C₅₋₁₀ heterocyclyl wherein said phenyl, benzyl or C₅₋₁₀ heterocyclyl group can either be unsubstituted or substituted with 1-3 substituents and each substituent is selected from the group consisting of hydroxyl, halogen, cyano, methyl, ethyl, propyl, isopropyl and trifluoromethyl.

22. A method as claimed in claim 8 wherein R⁸ is selected from the group consisting of phenyl, benzyl and C₅₋₁₀ heterocyclyl wherein said phenyl, benzyl or C₅₋₁₀ heterocyclyl group can either be unsubstituted or substituted with 1-3 substituents and each substituent is selected from the group consisting of hydroxyl, halogen, cyano, methyl, ethyl, propyl, isopropyl and trifluoromethyl.
